(19) United States
(12) Patent Application Publication
(10) Pub. No.: US 2026/0299409 A1
(43) Pub. Date: Oct. 1, 2026

(54) PHOTOACTIVE COMPOUNDS

(71) Applicant: Merck Patent GmbH, Darmstadt (DE)

(72) Inventors: Hung-Yang Chen, Basking Ridge, NJ (US); Chunwei Chen, Whitehouse Station, NJ (US); Ionela Daniela Carja, Darmstadt (DE); Ralph R. Dammel, Springfield, PA (US)

(21) Appl. No.: 19/473,032

(22) PCT Filed: Apr. 25, 2024
(86) PCT No.: PCT/EP2024/061393

§ 371 Date: Oct. 6, 2025

Related U.S. Application Data
(60) Provisional application No. 63/498,617, filed on Apr. 27, 2023.

(51) Int. Cl. G03F 7/004; C07D 221/14; G03F 7/16; G03F 7/32

(52) U.S. Cl. CPC G03F 7/0045; C07D 221/14; G03F 7/162; G03F 7/322

(57) ABSTRACT

A covalent compound free of any fluoroalkyls, any perfluoroalkyls or mixtures of fluoroalkyls and perfluoroalkyls comprising an imide N-(carbonylcarbamido) aryl sulfate moiety. In one of this compound it has structure (I), wherein R₁ is a linking group selected from the group consisting of an unsubstituted alkylene, a substituted alkylene, an unsubstituted vinylene, a substituted vinylene, an unsubstituted arylene, and a substituted arylene, where these linking groups are free of any fluoroalkyls, any perfluoroalkyls or mixtures of fluoroalkyls and perfluoroalkyls, and R₂ is an unsubstituted aryl or a substituted aryl which are free of any fluoroalkyls, any perfluoroalkyls or mixtures of fluoroalkyls and perfluoroalkyls and np is 1, 2, 3, 4 or 5. A positive or negative chemically amplified photoresist composition comprising as a PAG component said covalent compound. A process of forming a positive or negative photoresist patter respectively using said positive or negative chemically amplified photoresists.

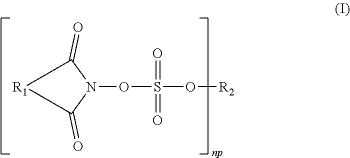

### FIELD OF THE INVENTION

The present invention relates to novel photoactive compounds useful in photoresist compositions in the field of microlithography, and especially useful for imaging negative and positive patterns in the production of semiconductor devices, as well as photoresist compositions and processes for imaging photoresists.

### BACKGROUND

Photoresist compositions are used in microlithography processes for making miniaturized electronic components such as in the fabrication of computer chips and integrated circuits. Generally, in these processes, a thin coating of film of a photoresist composition is first applied to a substrate material, such as silicon wafers used for making integrated circuits. The coated substrate is then baked to evaporate any solvent in the photoresist composition and to fix the coating onto the substrate. The photoresist coated on the substrate is next subjected to an image-wise exposure to radiation.

The radiation exposure causes a chemical transformation in the exposed areas of the coated surface. Visible light, ultraviolet (UV) light, electron beam and X-ray radiant energy are radiation types commonly used today in microlithographic processes. After this image-wise exposure, the coated substrate is treated with a developer solution to dissolve and remove either the radiation exposed or the unexposed areas of the photoresist. The trend toward the miniaturization of semiconductor devices has led to the use of new photoresists that are sensitive at lower and lower wavelengths of radiation and has also led to the use of sophisticated multilevel systems to overcome difficulties associated with such miniaturization.

There are two types of photoresist compositions: negative-working and positive-working. The type of photoresist used at a particular point in lithographic processing is determined by the design of the semiconductor device. When negative-working photoresist compositions are exposed image-wise to radiation, the areas of the photoresist composition exposed to the radiation become less soluble to a developer solution (e.g., a cross-linking reaction occurs) while the unexposed areas of the photoresist coating remain relatively soluble to such a solution. Thus, treatment of an exposed negative-working resist with a developer causes removal of the non-exposed areas of the photoresist coating and the creation of a negative image in the coating, thereby uncovering a desired portion of the underlying substrate surface on which the photoresist composition was deposited.

On the other hand, when positive-working photoresist compositions are exposed image-wise to radiation, those areas of the photoresist composition exposed to the radiation become more soluble to the developer solution (e.g., a rearrangement reaction occurs) while those areas not exposed remain relatively insoluble to the developer solution. Thus, treatment of an exposed positive-working photoresist with the developer causes removal of the exposed areas of the coating and the creation of a positive image in the photoresist coating. Again, a desired portion of the underlying surface is uncovered.

Photoresist resolution is defined as the smallest feature, which the resist composition can transfer from the photomask to the substrate with a high degree of image edge acuity after exposure and development. In many leading-edge manufacturing applications today, photoresist resolution on the order of less than one-half micron is necessary. In addition, it is almost always desirable that the developed photoresist wall profiles be near vertical relative to the substrate. Such demarcations between developed and undeveloped areas of the resist coating translate into accurate pattern transfer of the mask image onto the substrate. This becomes even more critical as the push toward miniaturization reduces the critical dimensions on the devices. In cases where the photoresist dimensions have been reduced to below 150 nm, the roughness of the photoresist patterns has become a critical issue. Edge roughness, commonly known as line edge roughness, is typically observed for line and space patterns as roughness along the photoresist line, and for contact holes as side wall roughness. Edge roughness can have adverse effects on the lithographic performance of the photoresist, especially in reducing the critical dimension latitude and in transferring the line edge roughness of the photoresist to the substrate. Hence, photoresists that minimize edge roughness are highly desirable.

Photoresists sensitive to short wavelengths, between about 100 nm and about 300 nm are often used where sub-half micron geometries are required. Particularly preferred are photoresists comprising non-aromatic polymers, a photoacid generator, optionally a dissolution inhibitor, and solvent.

High resolution, chemically amplified, deep ultraviolet (100-300 nm) positive and negative tone photoresists are available for patterning images with less than quarter micron geometries. To date, there are three major deep ultraviolet (UV) exposure technologies that have provided significant advancement in miniaturization, and these use lasers that emit radiation at 248 nm, 193 nm and 157 nm. Additionally, at the current forefront, extreme ultraviolet (EUV) lithography at 13.5 nm using a laser-pulsed tin droplet plasma is being used to pattern photoresists.

For use at UV, deep UV and EUV positive and negative chemically amplified photoresists comprise a polymer or oligomer, a photoacid generator and an organic spin casting solvent. In positive photoresists the polymer or oligomer is one which undergoes a catalytic deprotection of these materials under the influence of a photoacid generated from the PAG when irradiating a cast film of this composition on a substrate. Conversely, in negative photoresists the polymer or oligomer is one which becomes crosslinked catalytically under the influence of the photoacid and renders the cast film insoluble in the areas irradiated. The PAG is a photoactive component which generates an acid under the influence of radiation, either directly through the absorption of UV or deep UV light directly by the PAG, indirectly through the intermediacy of a sensitizer, or in the case of EUV through the action of a secondary electron generated by the EUV radiation.

Photoresists for 248 nm have typically been based on substituted polyhydroxystyrene and its copolymers, such as those described in U.S. Pat. Nos. 4,491,628, 5,350,660. On the other hand, photoresists for 193 nm exposure require non-aromatic polymers since aromatics are opaque at this wavelength. U.S. Pat. No. 5,843,624 and GB 2,320,718 disclose photoresists useful for 193 nm exposure. Generally, polymers containing alicyclic hydrocarbons are used for photoresists for exposure below 200 nm. Alicyclic hydrocarbons are incorporated into the polymer for many reasons, primarily since they have relatively high carbon to hydrogen ratios which improve etch resistance, they also provide transparency at low wavelengths, and they have relatively high glass transition temperatures. Photoresists sensitive at 157 nm have been based on fluorinated polymers, which are known to be substantially transparent at that wavelength. Photoresists derived from polymers containing fluorinated groups are described in WO 00/67072 and WO 00/17712.

The polymers used in a photoresist are designed to be transparent to the imaging wavelength, but on the other hand, the photoactive component has been typically designed to be absorbing at the imaging wavelength to maximize photosensitivity. The photosensitivity of the photoresist is dependent on the absorption characteristics of the photoactive component, the higher the absorption, the less the energy required to generate the acid, and the more photosensitive is the photoresist.

Photoacid generators (PAGs) are key components in chemically amplified resists used in photolithography. Per-fluoroalkyl sulfonate (PFAS), such as perfluorobutanesulfonates (PFBS), perfluorooctanesulfonates (PFOS) and other perfluoroalkylsulfonates (PFAS) have been well adopted as PAGs due to their strong acidity (superacid). Recently, concerns have been raised about their environmental impact due to their chemical persistence, bioaccumulation, and toxicity. It is a general interest to find environmental-friendly PAGs that are free of any fluoroalkyls, any perfluoroalkyls or mixtures of fluoroalkyls and perfluoroalkyls.

There are some commercialized fluorine-free PAGs using alkylsulfonic acid and tosyl sulfonic acid as the anion to generate acid. However, those PAGs are not strong acids (superacids) due to the lack of electron-withdrawing character like fluorine to stabilize the acid anion and make the acid stronger. This limits the application of these fluorine-free PAGs in photoresists that utilize high activation energy polymers.

Li et al. US 20090181319 A1 reported fluorine-free PAGs using electron withdrawing moiety such as CN, NO, NO₂, F, Cl, Br, I, SO₂Me, or CHO on the phenyl ring connected with sulfonic acid. These PAGs are applied for ArF(193 nm) lithography using sulfonium and onium cationic components. In J. Photopolym. Sci. Technol., p.173-p.183, Vol. 23, No. 2, 2010 IBM reported fluorine-free PAGs using cyano-group as the acceptor-substituted aromatic anions; pentacyanocyclopentadienide (CN5) and methoxycarbonyl-tetra-cyanocyclopentadienide (CN4-Cl). However, the toxicity of cyano-group and their stability in photoresist during the operation in lithography may still be a concern for their commercialization.

### DETAILED DESCRIPTION OF DRAWINGS

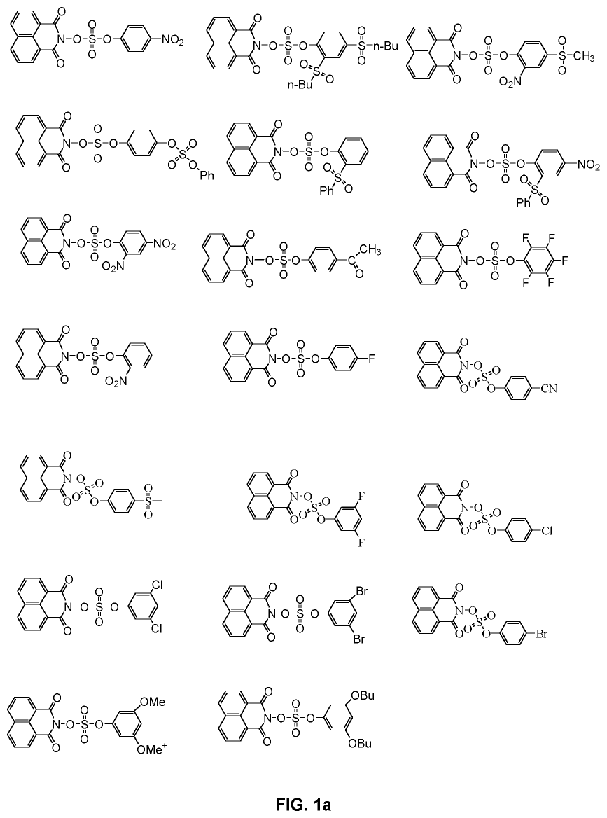

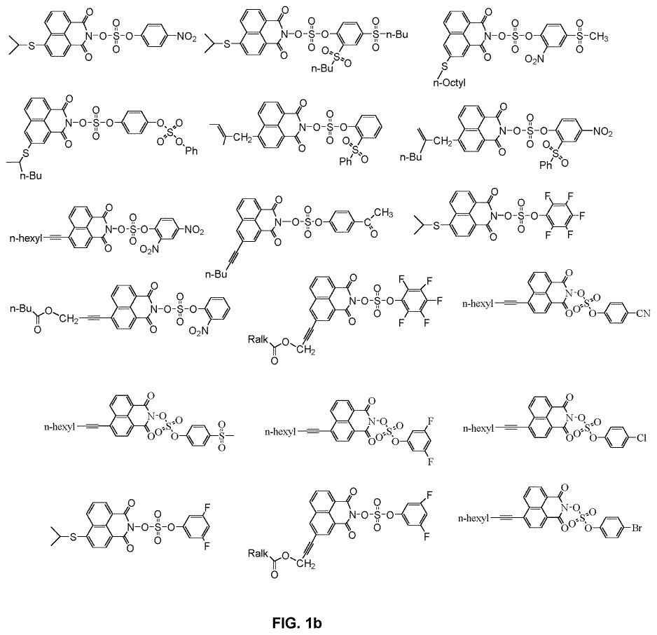

FIG. 1a Examples of naphthaleneimido sulfate containing inventive PAGs with electron withdrawing substituents on the phenyl moiety.

FIG. 1b Examples of naphthaleneimido sulfate containing inventive PAGs which incorporate both a UV sensitivity moiety on the naphthalene chromophore and an electron withdrawing substituent on the phenyl moiety.

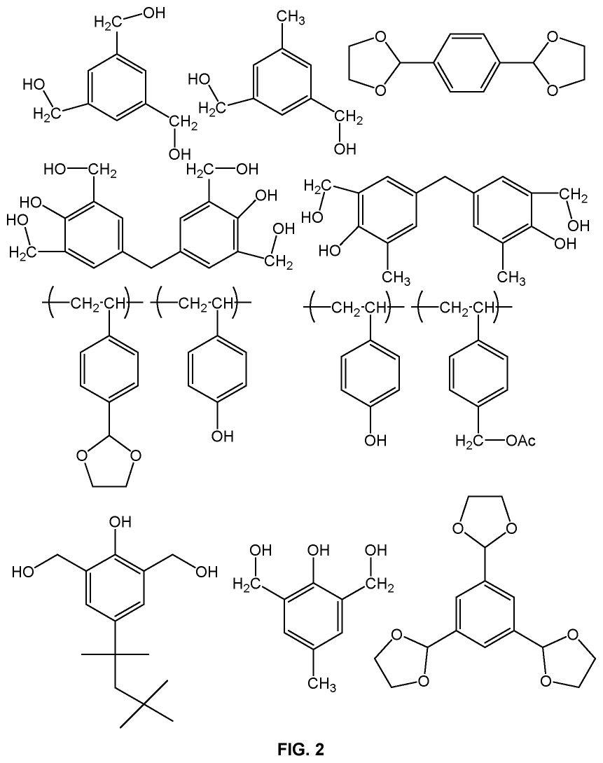

FIG. 2 Examples of polymers and crosslinkers which contain latent electrophiles.

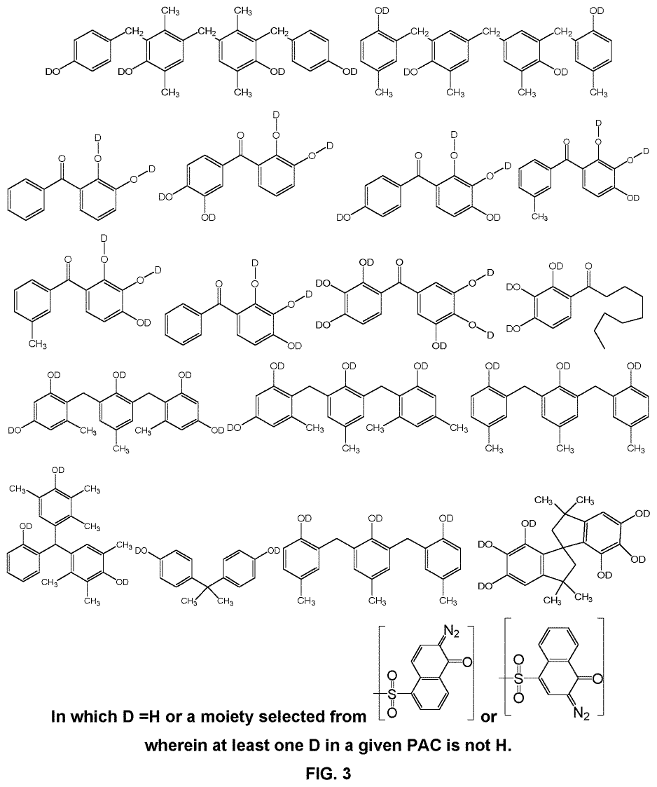

FIG. 3 Shows non-limiting examples of DNQ PAC compounds which may be used a free PAC component and/or be used to form a PAC moiety attached the polymer component on a phenolic moiety through an acetal comprising linking group

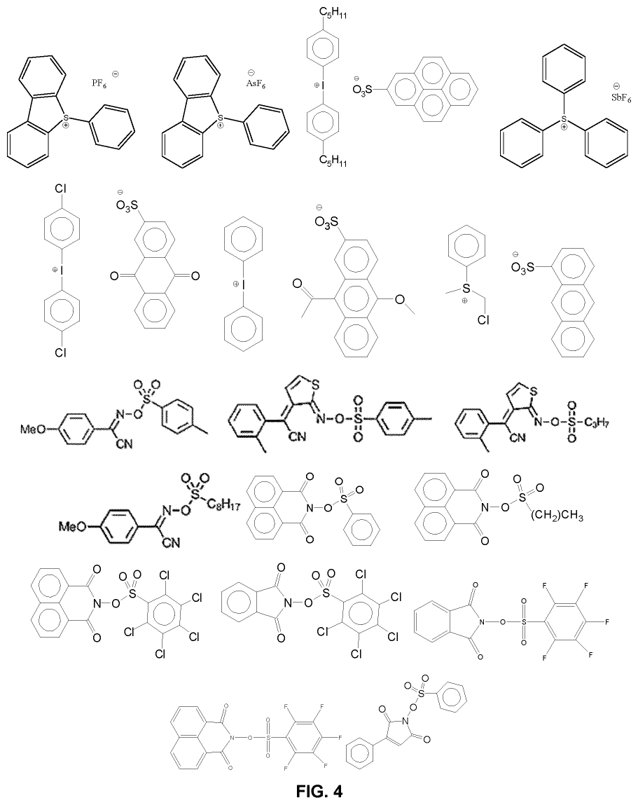

FIG. 4 Example of optional photoacid generators which generate sulfonic acids.

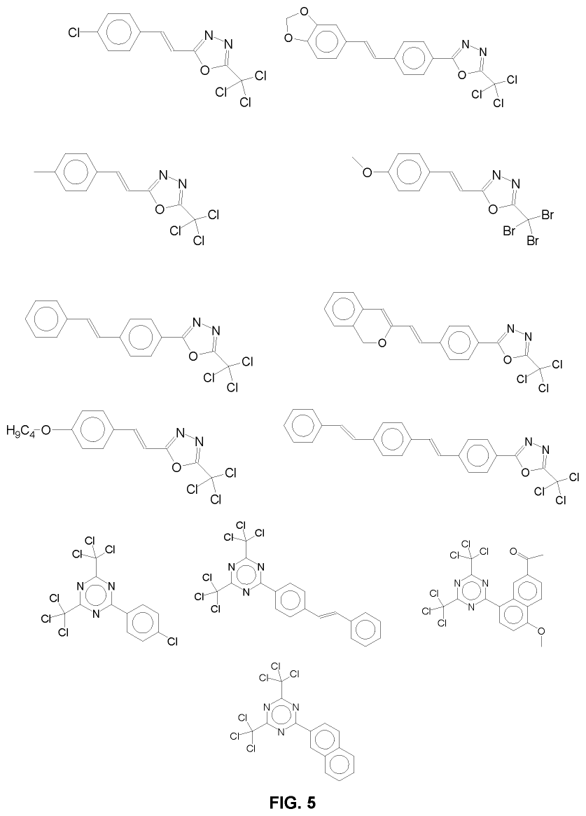

FIG. 5 Example of optional photoacid generators which generate HCl or HBr.

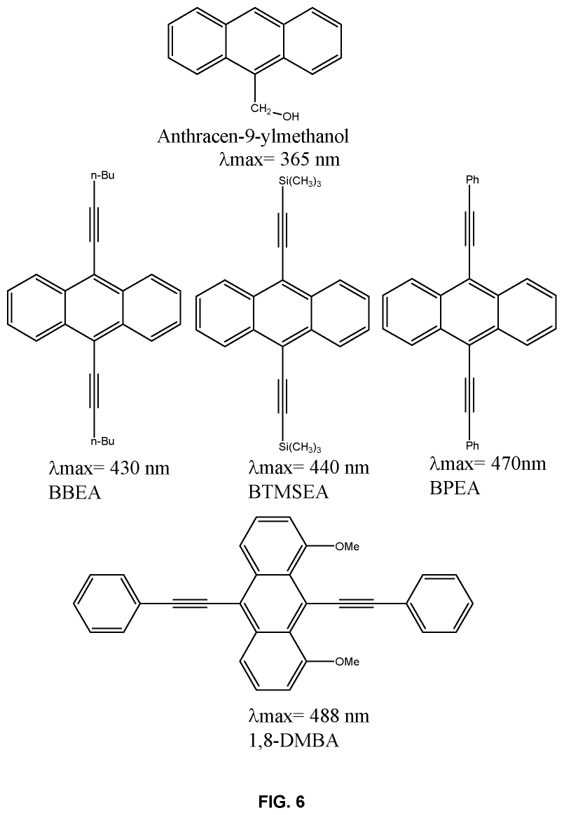

FIG. 6 Structures and maximum wavelength absorbance of specific representative sensitizers.

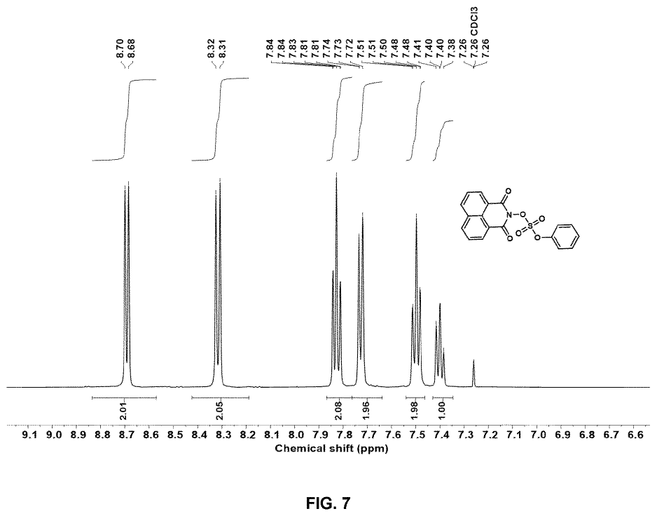

FIG. 7 ¹H NMR of PAG 1.

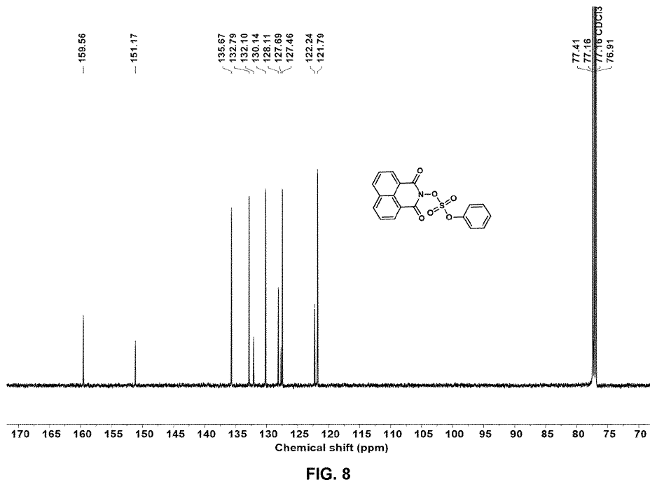

FIG. 8 ¹³C NMR of PAG 1.

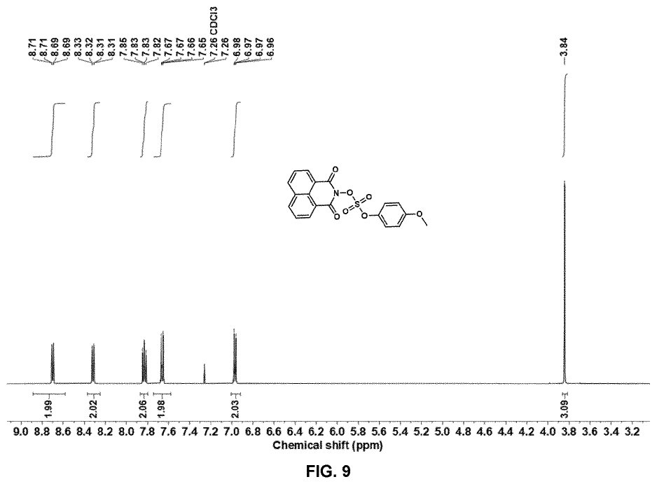

FIG. 9 ¹H NMR of PAG 2.

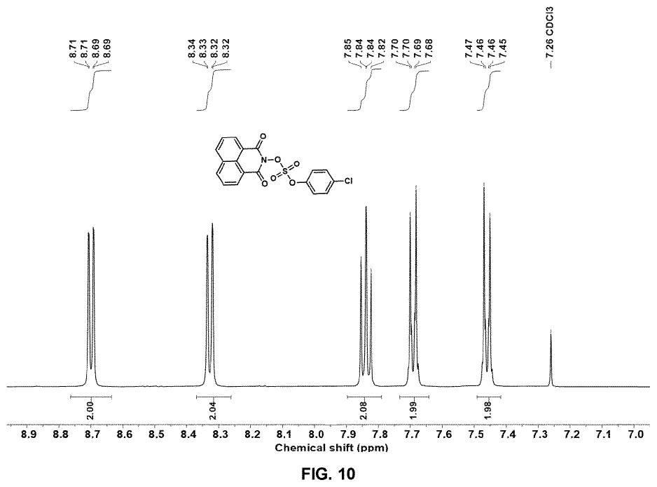

FIG. 10 ¹H NMR of PAG 3.

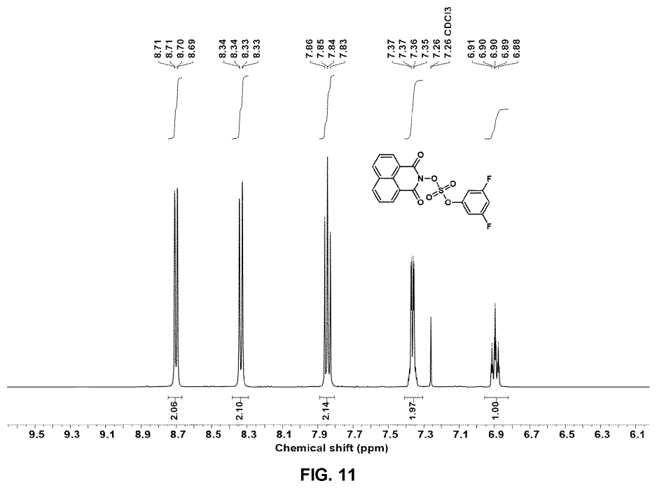

FIG. 11 ¹H NMR of PAG 4.

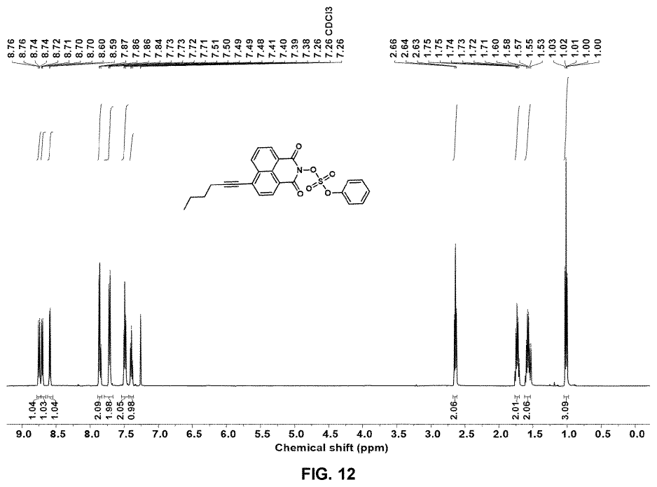

FIG. 12 ¹H NMR of PAG 5.

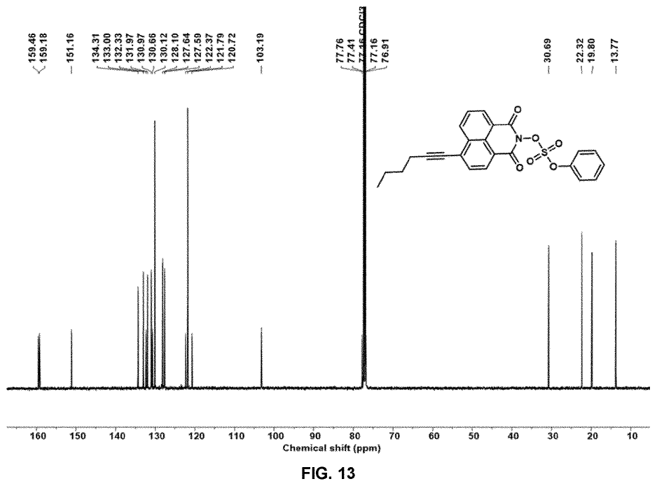

FIG. 13 ¹³C NMR of PAG 5.

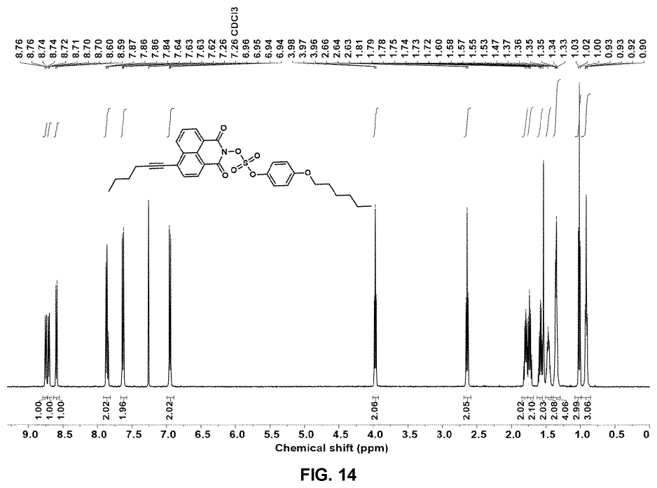

FIG. 14 ¹H NMR of PAG 6.

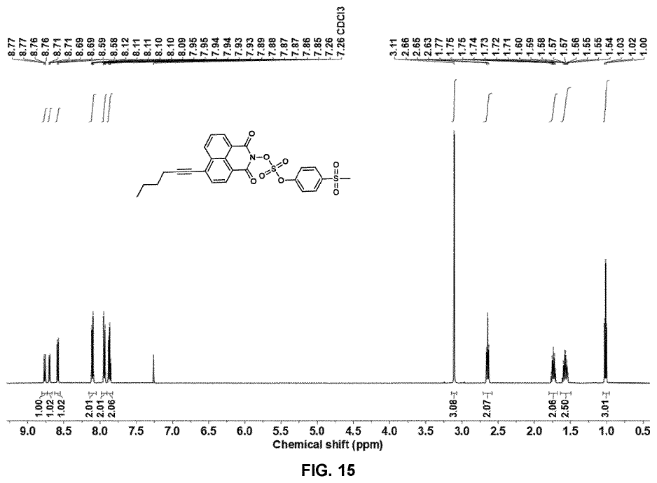

FIG. 15 ¹H NMR of PAG 7.

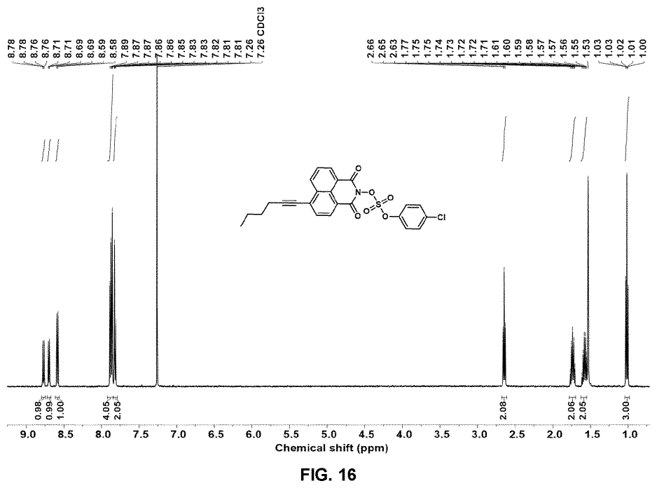

FIG. 16 ¹H NMR of PAG 8.

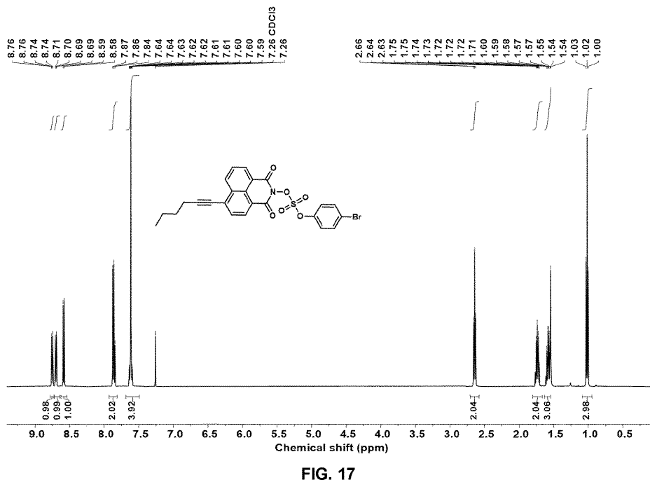

FIG. 17 ¹H NMR of PAG 9.

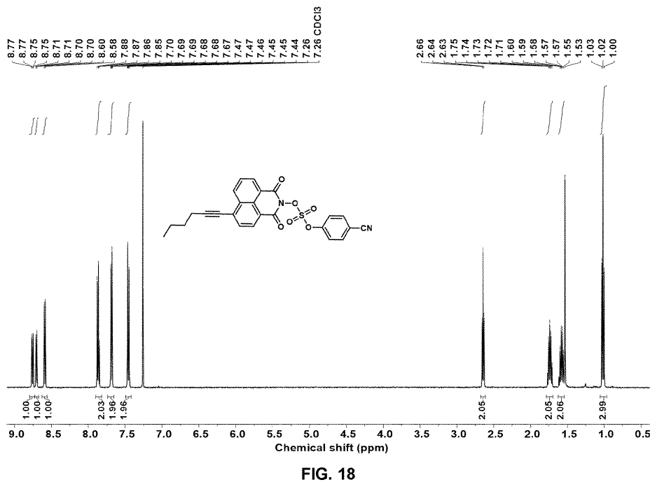

FIG. 18 ¹H NMR of PAG 10.

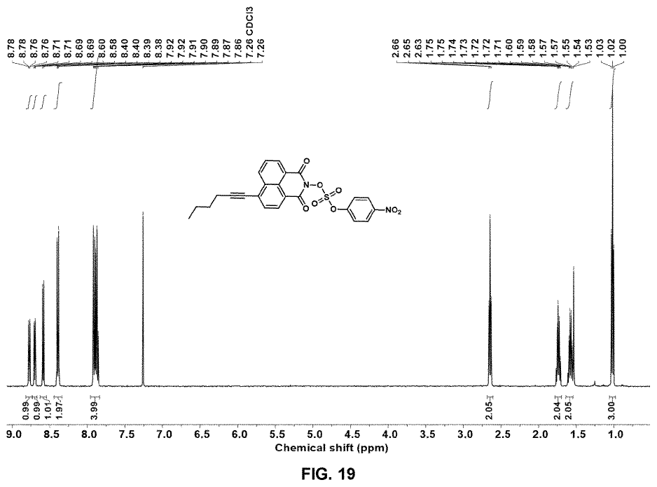

FIG. 19 ¹H NMR of PAG 11.

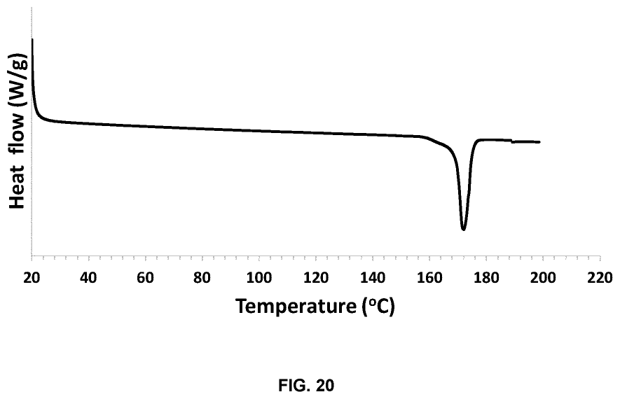

FIG. 20 DSC of PAG 1.

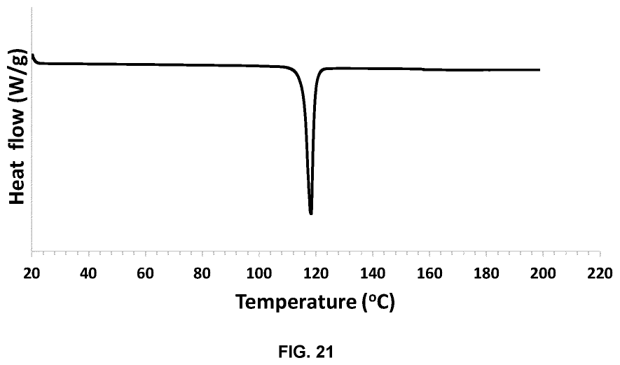

FIG. 21 DSC of PAG 5.

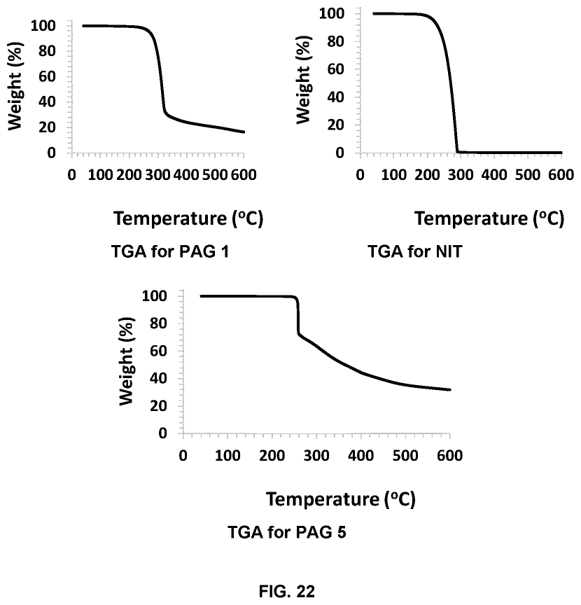

FIG. 22 Comparison of TGA of PAG 1, PAG 5 and NIT PAG.

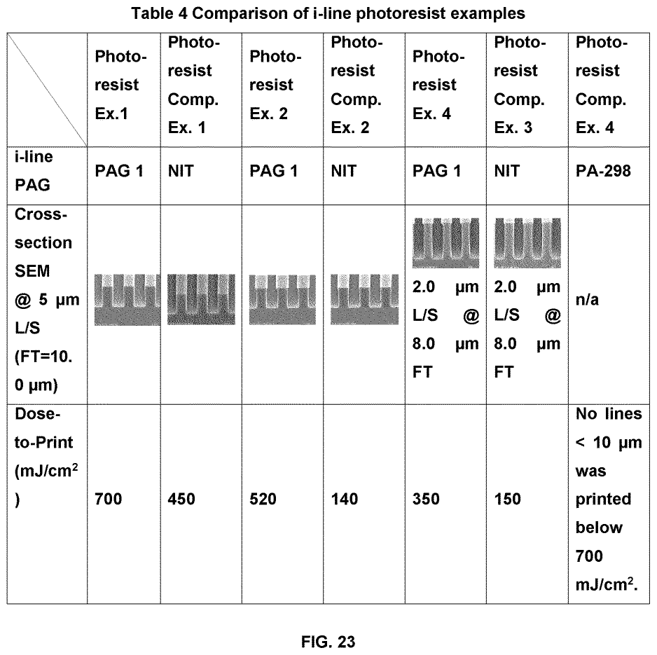

FIG. 23 Table 4 which compares the lithographic performance of Photoresist Examples 1 and 2 (Photoresist Ex. 1 and 2) to that of Photoresist comparative Examples 1 to 4 (Photoresist Comp. Ex 1 to 4).

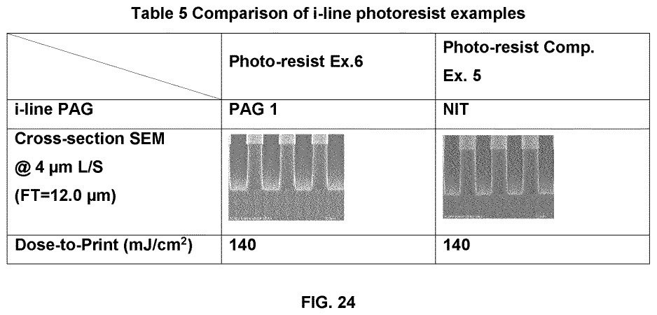

FIG. 24 Table 5 Comparison of Photoresist example 6 formulated with the inventive PAG 1, and Comparative Example 5 formulated with NIT PAG

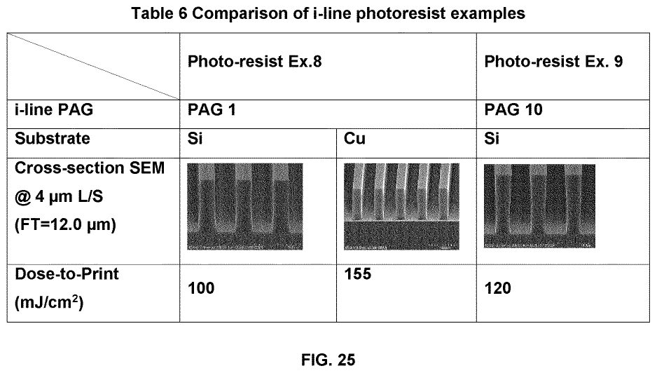

FIG. 25. Table 6 Lithographic Performance of Photoresist Example 8 on Si and Cu substrate and Photoresist Example 9 (PAG 10) on Si substrate.

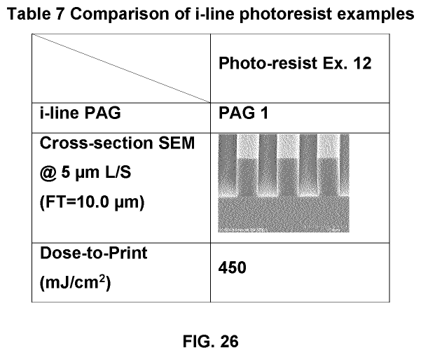

FIG. 26 Table 7 Lithographic performance of Photoresist Example 12. (Photoresist Example 12 shows a faster photospeed than its similar formulation Photoresist Examples 1)

### SUMMARY OF INVENTION

One aspect of this invention is a new class of photoacid generators (PAGs) free of any fluoroalkyls, any perfluoroalkyls or mixtures of fluoroalkyls and perfluoroalkyls, comprising at least one imide N-(carbonylcarbamido) aryl sulfate, which upon irradiation forms a very strong acid (superacid) which is free of any fluoroalkyls, any perfluoroalkyls or mixtures of fluoroalkyls and perfluoroalkyls, which has an easy synthesis route, and good solubility in organic spin casting solvents. In one embodiment of these PAGs they have structure (I), In one embodiment these have structure (I), wherein R₁ is a linking group selected from the group consisting of an unsubstituted alkylene, a substituted alkylene, an unsubstituted vinylene, a substituted vinylene, an unsubstituted arylene, and a substituted arylene, where these linking groups are free of any fluoroalkyls, any perfluoroalkyls or mixtures of fluoroalkyls and perfluoroalkyls, and R₂ is an unsubstituted aryl or a substituted aryl which are free of any fluoroalkyls, any perfluoroalkyls or mixtures of fluoroalkyls and perfluoroalkyls and np is the number of N-(carbonylcarbamido) sulfate moieties attached to an unsubstituted aryl or a substituted aryl which is either 1, 2, 3, 4 or 5. Other aspects of this invention are negative or positive chemically amplified photoresist compositions and the process of using these in forming an image on a substrate.

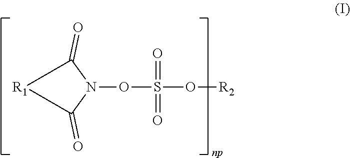

### DETAILED DESCRIPTION OF THE INVENTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or,” unless specifically stated otherwise. Furthermore, the use of the term “including,” as well as other forms such as “includes” and “included,” is not limiting. Also, terms such as “element” or “component” encompass both elements and components comprising one unit and elements or components that comprise more than one unit, unless specifically stated otherwise. As used herein, the conjunction “and” is intended to be inclusive and the conjunction “or” is not intended to be exclusive unless otherwise indicated. For example, the phrase “or, alternatively” is intended to be exclusive. As used herein, the term “and/or” refers to any combination of the foregoing elements including using a single element.

The term “alicyclic” refers to compound containing one or more carbon chains forming rings which can be saturated hydrocarbons or may contain unsaturation but are not aromatic.

The term C-1 to C-4 alkyl embodies methyl and C-2 to C-4 linear alkyls and C-3 to C-4 branched alkyl moieties and C-3 to C-4 alicyclic moieties, for example as follows: methyl, (—CH₃), ethyl (—CH₂—CH₃), n-propyl (—CH₂—CH₂—CH₃), isopropyl (—CH(CH₃)₂, n-butyl (—CH₂—CH₂—CH₂—CH₃), tert-butyl (—C(CH₃)₃), isobutyl (CH₂—CH(CH₃)₂, 2-butyl (—CH(CH₃)CH₂—CH₃), cyclopropyl, cyclobutyl. Similarly, the term C-1 to C-8 embodies methyl, C-2 to C-8 linear, C-3 to C-8 branched alkyls, C-3 to C-8 alicyclic moieties which encompass 1 or more rings (e.g., cyclopentyl, cyclohexyl, such as bicyclo[2.2.1]hep-2-yl, bicyclo[2.2.1]hep-1-yl, bicyclo[2.2.2.]oct-2-yl, and the like), C-5-C-8 alkylenecycloalkyls, and C-3 to C-8 alicyclic (e.g. cyclopropyl, cyclopentyl, cyclohexyl, bicyclic such as bicyclo[2.2.1]hep-2-yl, bicyclo[2.2.1]hep-1-yl, bicyclo[2.2.2.]oct-2-yl, bicyclo[2.2.2.)oct-1-yl and the like). Also, the term “C-1 to C-18 alkyl” includes methyl, C-2 to C-18 linear, C-3 to C-18 branched alkyls, C-4 to C-18 cycloalkyls, C-5-C-18 alkylenecycloalkyls C-3 to C-18 alicyclic (e.g. cyclopropyl, cyclobuty, cyclopentyl, cyclohexyl, alicyclic (e.g. cyclopropyl, cyclopentyl, cyclohexyl, bicyclic such as bicyclo[2.2.1]hep-2-yl, bicyclo[2.2.1]hep-1-yl, bicyclo[2.2.2.]oct-2-yl, bicyclo[2.2.2.)oct-1-yl adamantan-1-yl, adamatan-2-yl, octahydro-1H-4,7-methanoinden-5-yl, and the like. Similarly, the terms C-1 to C-18 alkyl and within them the aforementioned scope as described for the smaller carbon ranges extended to C-18 carbons instead for examples C-4 carbons or C-8 carbons. Additionally, these alkyl groups may be unsubstituted or substituted. Additionally, for alkyl groups which have 2 or more carbons which are substituted on the Naphthalene portion of the Naphthalene imide phenyl sulfate based photoacid PAG as described herein, these alkyl groups may additionally comprise a moiety within their length selected from a carbon to carbon double bond (=), a carbon to carbon triple bond (≡), a heteroatom comprising moiety selected from —O—, —S—, —C(═O)—, —C(═S)—, —S(═O)₂—, —S(═O)—, —C(═O)—O—, —C(═O)—S—, —O—S(═O)₂—, —O—C(═O)—O—, —C(═O)—NH—, —O—C(═O)—NH—, and —C(═O)—NR—, and mixtures thereof, where R is a C-1 to C-18 alkyl.

The term “C-2 to C-5 alkylene” embodies C-2 to C-5 linear alkylene moieties (e.g., ethylene, propylene etc.) and C-3 to C-5 branched alkylene moieties (e.g., —CH(CH₃)—, —CH(CH₃)—CH₂—, etc.).

The term “substituted” when designating an alkyl or alkylene moiety, within the inventive PAG compounds described herein, entails that substituents such as —O-Rw, hydroxy, —NO₂, —CN, —S(═O)₂-Rw, —O—S(═O)₂-Rw, —O—S(═O)₂-Rw, —O—S(═O)₂—O-Rw1, —O—S(═O)-Rw, —O—S(═O)—O-Rw1, —Br, —Cl, —I, —C(═O)—H, —C(═O)-Rw, —C(═O)—O-Rw, —O—C(═O)—ORw, —O—C(═O)-Rw, —S—R, —C(═O)—S-Rw, —S-Rw, —C(═S)-Rw, —C(═S)—O-Rw, —C(═S)—S-Rw, —O—C(═S)—S-Rw, an alkynyl, an alkenyl and the like, (where Rw is an unsubstituted or substituted alkyl or aryl moiety, Rw1 is a substituted or unsubstituted aryl moiety), and other similar substituent may be present but excludes substituents which have excessive basicity or acidity which would impede the function of the PAG, and specifically excludes the presence of F, fluorinated alkyls, perfluorinated alkyls or any other substituents which would contain within fluorinated alkyls, or perfluorinated alkyls, but does not exclude substituents which contain a fluorinated aryl, fluorinated arylene, and a perfluorinated aryl because such moieties do not contains any fluorinated alkyl or perfluorinated alkyls.

The terms an unsubstituted C-1 to C-18 alkoxy, a substituted C-1 to C-18 alkoxy an unsubstituted C-1 to C-18 alkylthio, a substituted C-1 to C-18 alkylthio, encompass within the scope for their alkyl moieties those as described above for unsubstituted C-1 to C-18 alkyl and substituted C-1 to C-18 alkyl.

The term “alkenyl” refers to a hydrocarbon containing a carbon-carbon double bond which at one carbon is attached to another moiety and at the other carbons has substituents Rₐₗₖₑₙ, R₁ₐₗₖₑₙ, R₂ₐₗₖₑₙ, which are individually selected from, a hydrogen atom, an aryl, or a C-1 to C-18 alkyl, similarly The term “alkynyl” refers to a hydrocarbon containing a carbon-carbon triple bond which at one carbon is attached to another moiety and at the other carbon has a substituent R_{alkyn} which is selected from, a hydrogen atom, an aryl, or a C-1 to C-18 alkyls. These respectively have the following structures:

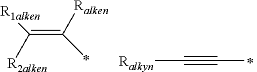

The term “aryl” or “aromatic groups” refers to such groups which contain 6 to 24 carbon atoms including phenyl, tolyl, xylyl, naphthyl, anthracenyl, biphenyls, bis-phenyls, tris-phenyls and the like. These aryl groups may further be substituted with any of the appropriate substituents, e.g., alkyl, alkoxy, acyl or aryl groups mentioned herein.

The term “fluoroalkyl” refers to a fluorinated alkyl or fluorinated alkylene moiety that contains at least one hydrogen (e.g. —CFH—, —CF₂H). The term “perfluorinated alkyl” refers to a fluorinated alkyl or fluoroalkylene moiety that does not contain any hydrogen atoms (e.g. —CF₂—, —CF₃). When the term free of any fluoroalkyls, any perfluoroalkyls or mixtures of fluoroalkyls and perfluoroalkyls, is employed herein this includes an alkyl moiety which contains perfluorinated alkyls, perfluorinated alkylenes, fluorinated alkyls, fluorinated alkylenes, and mixtures of these and also includes such moieties which incorporate within their length a carbon to carbon double bond (═), a carbon to carbon triple bond (≡), and/or a heteroatom comprising moiety selected from —O—, —S—, —C(═O)—, —C(═S)—, —S(═O)₂—, —S(═O)—, —C(═O)—O—, —C(═O)—S—, —O—S(═O)₂—, —O—C(═O)—O—, —C(═O)—NH—, —O—C(═O)—NH—, and —C(═O)—NR—, and mixtures thereof, where R is a C-1 to C-18 alkyl.

The term “arylene” refers to an aromatic hydrocarbon moiety which has two or more attachment points (e.g., 2-5), this moiety may be a single benzene moiety (e.g., two attachment points 1,4-phenylene, 1,3-phenylene and 1,2-phenylene; three attachment points 1,2,4-substituted benzene, 1,3,5-substituted benzene and the like), a polycyclic aromatic moiety with two attachment points such as those derived from naphthalene, anthracene, pyrene and the like, or multiple benzene rings in a chain which have two attachment point (e.g., biphenylene). In those instances where the aromatic moiety is a fused aromatic ring, these may be called fused ring arylenes, and more specifically named, for instance, naphthalenylene, anthracenylene, pyrenylene, and the like. Fused ring arylenes may be substituted or unsubstituted as described below, additionally these fused ring arylenes may also contain a hydrocarbon substituent which has two attachment sites on the fused ring forming an additional aliphatic or unsaturated ring forming by attachment to the fused ring a ring having 5 to 10 carbon atoms.

The terms “unsubstituted and substituted alkylene which have 2 or more carbons” as described above encompasses respectively, for each of these alkylene moiety types, ones which do contain any heteroatoms, heteroatom containing moieties, carbon to carbon double bond (═), or carbon to carbon triple bond (≡), unsaturation within the length of the alkylene moieties and also ones which do contain such moieties within the length of said alkylene moieties. Non limiting examples of heteroatoms and heteroatom containing groups which may be present in these alkylene moieties are —O—, —S—, —C(═O)—, —C(═S)—, —S(═O)₂—, —S(═O)—, —C(═O)—O—, —C(═O)—S—, —O—S(═O)₂—, —O—C(═O)—O—, —C(═O)—NH—, —O—C(═O)—NH—, and —C(═O)—NR—.

The term “substituted” when designating an aryl or arylene moiety, within the inventive PAG compounds described herein, unless otherwise indicated, entails that substituents such as a substituted aryl, an unsubstituted aryl, an unsubstituted C-1 to C-18 alkyl a substituted C-1 to C-18 alkyl, an unsubstituted C-1 to C-18 alkoxy, a substituted C-1 to C-18 alkoxy an unsubstituted C-1 to C-18 alkylthio, a substituted C-1 to C-18 alkylthio, O-Rw, hydroxy, —NO₂, —OH, —O-Rw, —CN, —S(═O)₂-Rw, —O—S(═O)₂-Rw, —O—S(═O)₂—O-Rw1, —O—S(═O)—R w, O—S(═O)—O-Rw1, —F, —Br, —Cl, —I, —C(═O)—H, —C(═O)-Rw, —C(═O)—O-Rw, —O—C(═O)—S—Rw, —O—C(═O)-Rw, —O—C(═O)—O-Rw, —S—R, —C(═O)—S-Rw, —S-Rw, —C(═S)-Rw, —C(═S)—O-Rw, —C(═S)—S-Rw, —O—C(═S)—S-Rw, an alkynyl, an alkenyl and other similar substituents, (where Rw is a C-1 to C-18 substituted or unsubstituted alkyl or aryl and Rw1 is a substituted or unsubstituted aryl), may be present but excludes substituents which have excessive basicity or acidity which would impede the function of the PAG, and specifically excludes fluorinated alkyls, perfluorinated alkyls or any other substituents which would contain within them fluorinated alkyls, or perfluorinated alkyls but does not exclude substituents which contain a fluorinated aryl, fluorinated arylene, a perfluorinated aryl and a perfluorinated arylene because such moieties do not contains any fluorinated alkyl or perfluorinated alkyls.

The term “electron donating substituent on aryl moieties” refers to substituents which are not or weakly electron withdrawing by induction and are electron donating by resonance for instance alkyl moieties. “Electron withdrawing substituents on aryl moieties” refers to substituents which are strongly electron withdrawing by induction because their attaching point is through an electronegative atom or moiety or are electron withdrawing by resonance for instance here is a listing of electron withdrawing groups which are electron withdrawing by resonance or induction, —C(═O)-alkyl, —OH, —O-alkyl, —O—C(═O)-alkyl, —S(═O)₂-alkyl, —NO₂, —O—S(═O)₂-alkyl, —CN, —S-alkyl, —C(═S)-alkyl, —C(═S)—O-alkyl, —C(═S)—S-alkyl, —O—C(═S)—S-alkyl, an alkynyl, an alkenyl and similar moieties.

The term “acrylic” as used herein encompasses repeat unit derived from acrylate derivatives generally, for example one derived from acrylate derivatives having the following structure, wherein the alkyl moiety may be a C-1 to C-8 alkyl, and Xacryl is either H, a C-1 to C-4 alkyl Racryl₁ is H, a C-1 to C-18 alkyl, a C-2 to C-18 alkyleneoxyalkyl, a C-2 to C-18 alkylenehydroxy, and a -Rcleav, where Rcleav is either a low activation energy or high activation energy protecting group which can be cleaved by a photogenerated acid from a PAG:

The term “styrenic” as used herein encompasses repeat units derived from styrene derivative generally for examples one derived from styrene derivatives having the following structure wherein Xsty moiety is H or a C-1 to C-4 alkyl and the Rsty moiety is H, an OH, a —O-Rcleav, a C-1 to C-8 alkyl moiety where Rcleav is either a low activation energy or high activation energy protecting group which can be cleaved by a photogenerated acid from a PAG:

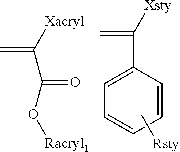

The term “Novolak” if used herein without any other modifier of structure, refers to Novolak resins which are soluble at room temperature in aqueous bases such as 0.26 N tetramethylammonium hydroxide and other aqueous developers of similar hydroxide normality.

The term “L/S,” is an abbreviation for line and space lithographic features.

This invention provides photoacid generators which are free of any fluoroalkyls, any perfluoroalkyls or mixtures of fluoroalkyls and perfluoroalkyls using aryl hydrogen sulfate as the generated super acid after exposure.

The term “covalent,” when referring to a compound or an organic moiety, has the meaning that this compound or this organic moiety only contains covalent bonds and does not contain any ionic bonds. For instance, a covalent photoacid generator (PAG), is one which only contains covalent bonds and does not contain any ionic bonds such as would occur in onium salt type photoacid generators such as a triphenylsulfonium or diphenyl iodonium salt of a strong acid such as a sulfonic acid.

Most PAGs typically incorporate a sulfonate group that, upon irradiation, generates a sulfonic acid. The strength of the corresponding acid primarily depends on the stability of its anion and the absence of well-defined protonation centers within it. In other words, a prerequisite in designing a strong acid is to assure a high degree of negative charge delocalization in its anion. Sulfo-anions effectively combine a polarizable sulfur atom with the high electron-acceptor tendency of the ═O atoms. The negative charge can be further delocalized through a judicious selection of the organic substrate linked to the SO3- group. In PFAS anions, the delocalization is driven by the strong negative inductive effect from the adjacent CF₂ (or CF₃) groups. We have discovered that by introducing an additional oxygen atom next to sulfonic moiety, further delocalization is achieved, leading to the generation of stronger acids. For instance, phenyl sulfonic acid has a predicted pKa of −0.6, according to ACD/Labs, whereas phenyl hydrogen sulfate has a value of −4.3 according to the same database. At the same time, aryl sulfates have acidities which are higher than that of perfluoroalkyl sulfonic acid. Table 1 provides a comparison of the pKa values between various aryl hydrogen sulfates and triflic acid. Even the unsubstituted aryl hydrogen sulfate, phenyl hydrogen sulfate, exhibits a lower pKₐ value than that of triflic acid, indicating greater acidity. As shown in Table 1 a, substitution of the phenyl moiety in this aryl hydrogen sulfate acid, even with substituents which are electron donating by resonance in the para position, still results in significantly high levels of acidity. On the other hand, substitution at the meta position with substituents which are electron donating by resonance imparts even higher acidity compared to perfluoroalkyl sulfonic acids like triflic acid. Additionally, when an electron withdrawing substituent by resonance and induction, such as alkylsulfone substituent or nitro, is introduced at the para position, it imparts even greater acidity.

TABLE 1a

| Photoacid Formed | Predicted pKₐ (ACD/Labs)* |
|---|---|
| 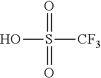 Triflic Acid | −3.9 |
| 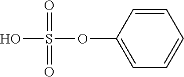 [SMILES: O=S(=O)(O)Oc1ccccc1] phenyl hydrogen sulfate | −4.3 |
| 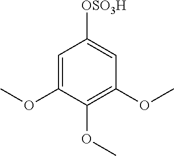 | −4.3 |
| 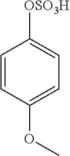 | −3.7 |
| 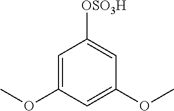 | −4.62 |
| 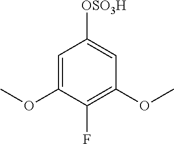 | −4.8 |
| 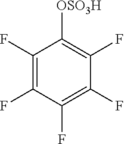 | −4.04 |
| 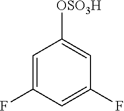 | −4.91 |
| 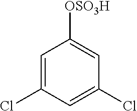 | −4.84 |
| 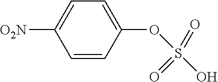 [SMILES: O=[N+]([O-])c1ccc(OS(=O)(=O)O)cc1] | −5.72 |
| 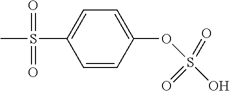 | −4.95 |
| 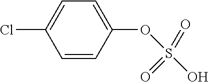 [SMILES: O=S(=O)(O)Oc1ccc(Cl)cc1] | −4.43 |

For instance, the predicted pKₐ of phenylhydrogen sulfate (pKa=−4.3) is comparable with the value of superacid triflic acid (pKₐ=−3.9). Naphthalene imide was utilized as the chromophore component for the application in i-line lithography. From the chemical structure point of view, this phenyl sulfate PAG (PAG 1) is relatively stable compared with the previous types of organic PAGs containing functional electron-withdrawing groups. The predicted low pKₐ as calculated by ACD/Labs*, is probably caused by the higher electronegativity of the oxygen atom (3.5) linked adjacent to the sulfur atom (2.5) in this acid. Table 1b compares NIT PAG which generates triflic acid to the corresponding Naphthalene imide phenyl sulfate based (PAG 1), which is one of the PAGs described herein. *(ACD/pKₐ software version 4.0 for Microsoft windows, Advanced Chemistry development Inc 8 King Street East, Suite 107, Toronto, Ontario Canada)

TABLE 1b

| Photoacid Generator | Photoacid Formed | Predicted pKₐ (ACD/Labs) |
|---|---|---|
| 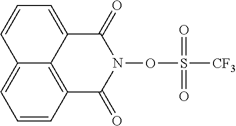 [SMILES: O=C1c2cccc3cccc(c23)C(=O)N1OS(=O)(=O)C(F)(F)F] NIT PAG | 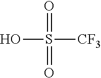 Triflic Acid | −3.9 |
| 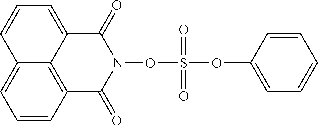 [SMILES: O=C1c2cccc3cccc(c23)C(=O)N1OS(=O)(=O)Oc1ccccc1] PAG 1 | 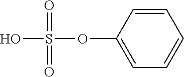 [SMILES: O=S(=O)(O)Oc1ccccc1] phenyl hydrogen sulfate | −4.3 |

PAG 1 in this invention was tested in different types of photoresist compositions and showed excellent lithographic performance that is comparable with references using the commercial perfluorocarbon (PFC)-containing NIT PAG (N-Hydroxynaphthalimide triflate). Further PAG 1 has strong i-line absorption, strong acid (superacid), easy synthesis, good solubility and was tested with both chemically amplified positive-type and negative-type photoresist formulations.

The CAR (Chemically amplified resist) formulations were spin coated on a substrate, soft baked on a hotplate, exposed with i-line stepper by a mask, then post-exposure-bake (PEB) and developed with aqueous alkaline solution. Finally, the wafers were rinsed with DI water and then spin dried to obtain photoresist patterns.

### Compounds

One aspect of this invention is a new class of covalent photoacid generators (PAGs) which are free of any fluoroalkyls, any perfluoroalkyls or mixtures of fluoroalkyls and perfluoroalkyls, and comprise at least one N-(carbonylcarbamido) sulfate attached to an aryl moiety forming an imide N-(carbonylcarbamido) aryl sulfate moiety, where the terms N-(carbonylcarbamido) sulfate and N-(carbonylcarbamido) sulfate moiety refers to the following structures where * designates an attachment point to a covalent organic moiety and “aryl” designates an aryl moiety as defined herein which may be unsubstituted or substituted. Non limiting examples of possible substituents are —NO₂, —OH, —O-Rw, —C(═O)-Rw, —C(═O)—H, —CN, —F, —Cl, —Br, —I, a C-1 to C-18 unsubstituted alkyl, a C-1 to C-18 substituted alkyl, phenyl, a substituted phenyl, —S(═O)₂-Rw, —O—S(═O)₂-Rw, —O—S(═O)₂—O-Rw1, —O—S(═O)-Rw, —O—S(═O)—O-Rw1, —C(═O)-Rw, —C(═O)—O-Rw, —C(═O)—S-Rw, —O—C(═O)—S-Rw, —O—C(═O)-Rw, —O—C(═O)—O-Rw, —S-Rw1, —C(═S)-Rw, —C(═S)—O-Rw, —C(═S)—S-Rw, —O—C(═S)—S-Rw, an alkynyl, an alkenyl and other similar electron withdrawing substituents, where Rw is a C-1 to C-18 substituted or unsubstituted alkyl or an unsubstituted or substituted aryl and Rw1 is an unsubstituted or substituted aryl. Another example of possible substitution is when the aryl moiety is substituted with at least one additional N-(carbonylcarbamido) sulfate moiety where ** designates the attachment point of this functional group to the aryl moiety in the N-(carbonylcarbamido) sulfate moiety and * designates the attachment point to a second organic moiety (shown below are examples of the resultant general structures which would result from mono, di and tri substitution).

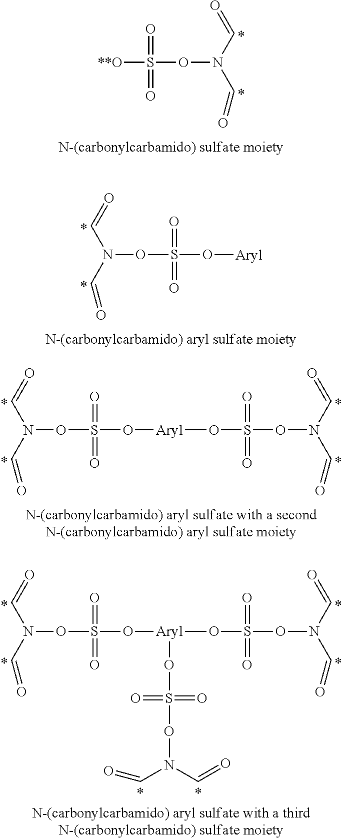

This PAG upon irradiation photogenerates a very strong acid (superacid) which is free of any fluoroalkyls, any perfluoroalkyls or mixtures of fluoroalkyls and perfluoroalkyls. These PAGs have an easy synthesis route, and also have good solubility in organic spin casting solvents.

In one embodiment of this invention, these new PAGs have structure (I), wherein R₁ is a linking group selected from the group consisting of an unsubstituted alkylene, a substituted alkylene, an unsubstituted vinylene, a substituted vinylene, an unsubstituted arylene, and a substituted arylene. These linking groups are free of any fluoroalkyls, any perfluoroalkyls or mixtures of fluoroalkyls and perfluoroalkyls, and R₂ is an unsubstituted aryl or a substituted aryl which is also free of any fluoroalkyls, any perfluoroalkyls or mixtures of fluoroalkyls and perfluoroalkyls and np is the number of N-(carbonylcarbamido) sulfate moieties attached to an unsubstituted aryl or a substituted aryl moieties R₂ which is either 1, 2, 3, 4 or 5. In one embodiment of this aspect np is 1, in another np is 2 in another it is 3 another it is 4, in yet another it is 5. In one aspect of these embodiments R₂ is a substituted or unsubstituted phenyl moiety; in another aspect of these embodiments R₂ has no further substituents; in another R₂ is further substituted with a substituent which is free of any fluoroalkyls, any perfluoroalkyls which is chosen from electron donating, electron withdrawing substituents or mixtures of such substituents,

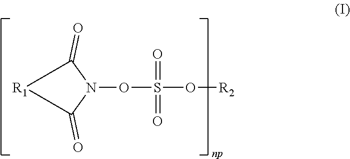

In another aspect of this invention, of said covalent compound which has structure (I), where R₁ is said unsubstituted alkylene or said substituted alkylene, it more specifically has structure (Ia), wherein R₁a, R₁b, R′₁a and R′₁b are independently selected from a hydrogen atom, a C-1 to C-18 alkyl, a C-1 to C-18 alkoxy, a substituted aryl, an unsubstituted aryl, and mixtures thereof, and np is 1, 2, 3, 4 or 5. In one embodiment of this aspect np is 1, in another np is 2 in another it is 3, in another it is 4, in yet another it is 5; in one aspect of these embodiments R₂ is a phenyl moiety; in another aspect of these embodiments R₂ has no further substituents; in another R₂ is further substituted with a substituent which is free of any fluoroalkyls, any perfluoroalkyls which is chosen from electron donating, electron withdrawing substituents or mixtures of such substituents.

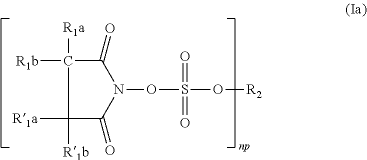

In another aspect of this invention, wherein said covalent compound has structure (I), where R₁ is said unsubstituted alkylene or said substituted alkylene, said compound has structure (Ib), wherein R₁a, R₁b, R₁c, R′₁a, R′₁b, R′₁c, are independently selected from a hydrogen atom, Cl, Br, —I, an unsubstituted C-1 to C-18 alkyl, a substituted C-1 to C-18 alkyl, an unsubstituted C-1 to C-18 alkoxy, a substituted C-1 to C-18 alkoxy, an unsubstituted aryl, a substituted aryl and mixtures thereof, and np is 1, 2, 3, 4 or 5. In one embodiment of this aspect np is 1, in another np is 2 in another it is 3, in another it is 4, in yet another it is 5. In one aspect of these embodiments R₂ is a phenyl moiety; in another aspect of these embodiments R₂ has no further substituents; in another R₂ is further substituted with a substituent which is free of any fluoroalkyls, any perfluoroalkyls which is chosen from an electron donating, an electron withdrawing substituents or a mixtures of such substituents.

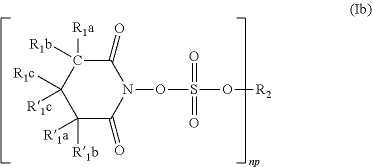

In another aspect of this invention, wherein said covalent compound has structure (I), where R₁ is said unsubstituted alkylene or said substituted alkylene, said compound has structure (Ic), wherein R₁a, R₁b, R₁c, R′₁a, R′₁b, R′₁c, are independently selected from a hydrogen atom, a C-1 to C-18 alkyl, a C-1 to C-18 alkoxy, a substituted aryl, an unsubstituted aryl, and mixtures thereof, and np is 1, 2, 3, 4 or 5. In one aspect of this embodiment R₁a, R₁b, R₁c, R′₁a, R′₁b, R′₁c are all a hydrogen atom. In another aspect of this embodiment at least one of R₁a, R₁b, R₁c, R′₁a, R′₁b, or R′₁c, is a C-1 to C-18 alkyl; in another aspect of this embodiment, it is a C-1 to C-8 alkyl, in another aspect of this embodiment it is a C-1 to C-4 alkyl. In another aspect of this embodiment at least one of R₁a, R₁b, R₁c, R′₁a, R′₁b, or R′₁c, is a C-1 to C-18 alkoxy; in another aspect of this embodiment, it is a C-1 to C-8 alkoxy, in another aspect of this embodiment it is a C-1 to C-4 alkoxy and np 1, 2, 3, 4 or 5. In one embodiment of this aspect np is 1, in another np is 2 in another it is 3, in another it is 4, in yet another it is 5. In one aspect of these embodiments R₂ is a phenyl moiety; in another aspect of these embodiments R₂ has no further substituents; in another R₂ is further substituted with a substituent which is free of any fluoroalkyls, any perfluoroalkyls which is chosen from an electron donating, an electron withdrawing substituents or a mixtures of such substituents.

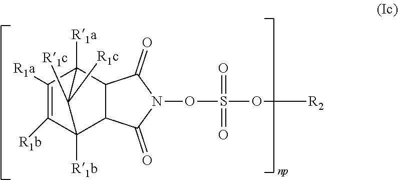

In another aspect of this invention, wherein said covalent compound has structure (I), where R₁ is said unsubstituted vinylene or said substituted vinylene, said compound has structure (Id), wherein Rid and R′₁d are independently selected from a hydrogen atom, Cl, Br, I, an unsubstituted C-1 to C-18 alkyl, a substituted C-1 to C-18 alkyl, an unsubstituted C-1 to C-18 alkoxy, a substituted C-1 to C-18 alkoxy, an unsubstituted aryl and a substituted aryl and mixtures thereof, and np is 1, 2, 3, 4 or 5. In one aspect of this embodiment Rid and R′₁d are both a hydrogen atom. In another aspect of this embodiment at least one of Rid and R′₁d is a C-1 to C-18 alkyl; in another aspect of this embodiment, it is a C-1 to C-8 alkyl, and in another aspect, it is a C-1 to C-4 alkyl. In another aspect of this embodiment at least one of Rid and R′₁d is a C-1 to C-18 alkoxy; in another aspect of this embodiment, it is a C-1 to C-8 alkoxy, and in another aspect it is a C-1 to C-4 alkoxy, and np is 1, 2, 3, 4 or 5. In one embodiment of this aspect np is 1, in another np is 2 in another it is 3; in another it is 4, in yet another it is 5. In one aspect of these embodiments R₂ is a phenyl moiety; in another aspect of these embodiments R₂ has no further substituents; in another R₂ is further substituted with a substituent which is free of any fluoroalkyls, any perfluoroalkyls which is chosen from an electron donating substituent, an electron withdrawing substituents or a mixtures of such substituents,

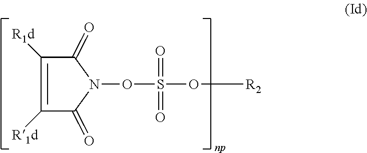

In another aspect of this invention, wherein said covalent compound has structure (I), where R₁ is said unsubstituted arylene or said substituted arylene, said compound has structure (Ie), wherein Rar₁, Rar₂, Rar₃, and Rar₄, are independently selected from a hydrogen atom, F, Cl, Br, I, an unsubstituted C-1 to C-18 alkyl, a substituted C-1 to C-18 alkyl, an unsubstituted C-1 to C-18 alkoxy, a substituted C-1 to C-18 alkoxy, an unsubstituted aryl, a substituted aryl and mixtures thereof, and np 1, 2, 3, 4 or 5. In one embodiment of this aspect np is 1, in another np is 2 in another it is 3 in another it is 4, and in yet another it is 5. In one aspect of these embodiments R₂ is a phenyl moiety; in another aspect of these embodiments R₂ has no further substituents; in another R₂ is further substituted with a substituent which is free of any fluoroalkyls, any perfluoroalkyls which is chosen from an electron donating, an electron withdrawing substituents or a mixtures of such substituents.

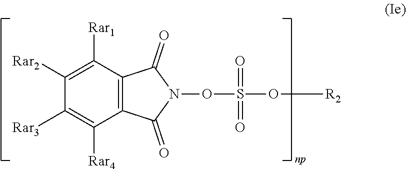

In another aspect of this invention, wherein said covalent compound has structure (I), where R₁ is said unsubstituted arylene or said substituted arylene, these are selected from a substituted fused polycyclic aromatic hydrocarbon moiety, or an unsubstituted fused polycyclic aromatic hydrocarbon moiety.

In another aspect of this invention, wherein said covalent compound has structure (I), where R₁ is an unsubstituted arylene or a substituted arylene, selected from a naphthalene moiety, an anthracene moiety, a phenanthrene moiety, a phenalene moiety, a tetracene moiety, a chrysene moiety a triphenylene moiety, a pyrene moiety, a pentacene moiety, and a perylene moiety, further wherein these moieties may be substituted or unsubstituted.

In another aspect of this invention, wherein said covalent compound has structure (I), where R₁ is a substituted or unsubstituted naphthalene moiety.

In another aspect of this invention, wherein said covalent compound has structure (I), where R₁ is a substituted or unsubstituted naphthalene moiety it has structure (If), where Rar₅, Rar₆, Rar₇, Rar₈, Rar₉, Rar₁₀, are independently selected from a hydrogen atom, F, Cl, Br, I, a substituted aryl, an unsubstituted aryl, an unsubstituted C-1 to C-18 alkyl a substituted C-1 to C-18 alkyl, an unsubstituted C-1 to C-18 alkoxy, a substituted C-1 to C-18 alkoxy an unsubstituted C-1 to C-18 alkylthio, a substituted C-1 to C-18 alkylthio, and mixtures thereof, where for substituents comprising C-2 to C-18 unsubstituted alkyl, and substituted C-2 to C-18 alkyl these may optionally comprise, within the length of the alkyl moiety, a moiety within their length selected from a carbon to carbon double bond (═), a carbon to carbon triple bond (≡), and a heteroatom comprising moiety selected from —O—, —S—, —C(═O)—, —C(═S)—, —S(═O)₂—, —S(═O)—, —C(═O)—O—, —C(═O)—S—, —O—S(═O)₂—, —O—C(═O)—O—, —C(═O)—NH—, —O—C(═O)—NH—, and —C(═O)—NR—, and mixtures thereof; where R is a C-1 to C-18 alkyl; and where np is 1, 2, 3, 4 or 5. In one embodiment of this aspect np is 1, in another np is 2 in another it is 3 in another it is 4, in yet another it is 5; in one aspect of this embodiment there are no further substituents on R₂; in another aspect, when np is 1 R₂ it contains 1 to 5 further substituents, when np is 2 it contains 1 to 4 further substituents, when np is 3 it contains 1 to 3 further substituents, when np is 4 it contains 1 to 2 further substituent, and when np is 5 it contains 1 further substituent. When further substituents are present these may be chosen from an electron donating, an electron withdrawing substituents or a mixtures of such substituents. In one aspect said substituents are independently selected from —NO₂, —OH, —O-Rw, —CN, —F, —Cl, —Br, —I, C-1 to C-18 unsubstituted alkyl, C-1 to C-18 substituted alkyl, —S(═O)₂-Rw, —O—S(═O)₂-Rw, —O—S(═O)₂—O-Rw1, —O—S(═O)-Rw, —O—S(═O)—O-Rw1, —C(═O)—O-Rw, —C(═O)—H, —C(═O)—S-Rw, —O—C(═O)—S-Rw-O—C(═O)—S—Rw-O—C(═O)—S-Rw, —O—C(═O)-Rw, —O—C(═O)—O-Rw, —S-Rw1, —C(═S)-Rw, —C(═S)—O-Rw, —C(═S)—S-Rw, —O—C(═S)—S-Rw, an alkynyl, an alkenyl and mixtures thereof, where Rw is a C-1 to C-18 substituted or unsubstituted alkyl or an unsubstituted or substituted aryl and Rw1 is an unsubstituted or substituted aryl.

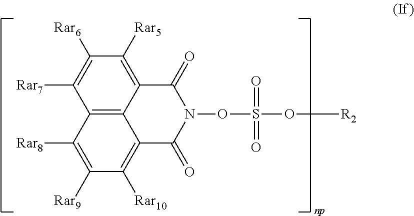

In one aspect of this invention, described herein where said covalent compound has either structures (I), (Ia), (Ib), (Ic), (Id), (Ie) or (If), R₂ is phenyl or a substituted phenyl. In one aspect of this embodiment R₂ is phenyl. In another aspect, R₂ is a further substituted phenyl in which, when np is 1 it contains 1 to 5 further substituents, when np is 2 it contains 1 to 4 further substituents, when np is 3 it contains 1 to 3 further substituents, when np is 4 it contain 1 to 2 further substituents, and when np is 5 it contains 1 further substituent independently selected from —NO₂, —OH, —O-Rw, —CN, —F, —Cl, —Br, —I, C-1 to C-18 unsubstituted alkyl, C-1 to C-18 substituted alkyl, phenyl, a substituted phenyl, —S(═O)₂—R, —O—S(═O)₂-Rw, —O—S(═O)₂—O-Rw1, —O—S(═O)-Rw, —O—S(═O)—O-Rw1, —C(═O)—H, —C(═O)-Rw, —C(═O)—O-Rw, —C(═O)—S-Rw, —O—C(═O)—S—Rw, —O—C(═O)-Rw, —O—C(═O)—O-Rw, —S-Rw1, —C(═S)-Rw, —C(═S)—O-Rw, —C(═S)—S-Rw, —O—C(═S)—S-Rw, an alkynyl, an alkenyl and mixtures thereof, where Rw is a C-1 to C-18 substituted or unsubstituted alkyl, or substituted or unsubstituted aryl and Rw1 is a substituted or unsubstituted aryl, where said substituents comprising an alkyl either comprise a moiety within their length selected from a carbon to carbon double bond (═), a carbon to carbon triple bond (≡), and a heteroatom comprising moiety selected from —O—, —S—, —C(═O)—, —C(═S)—, —S(═O)₂—, —S(═O)—, —C(═O)—O—, —C(═O)—S—, —O—S(═O)₂—, —O—C(═O)—O—, —C(═O)—NH—, —O—C(═O)—NH—, and —C(═O)—NR—, and mixtures thereof, where R is a C-1 to C-18 alkyl, or do not contain any such moieties within their length.

In another aspect of this invention, described herein where said covalent compound has either structures (I), (Ia), (Ib), (Ic), (Id), (Ie) or (If), R₂ is a further substituted phenyl, which when np is 1 it contains 1 to 5 further substituents, when np is 2 it contains 1 to 4 further substituents, when np is 3 it contains 1 to 3 further substituents, when np is 4 it contain 1 to 2 further substituents, and when np is 5 it contains 1 further substituent, independently selected from —NO₂, —OH, —O-Rw, —CN, —F, —Cl, —Br, —I, —S(═O)₂-Rw, —O—S(═O)₂-Rw, —O—S(═O)₂—O-Rw1, —O—S(═O)-Rw, —O—S(═O)—O-Rw1, —C(═O)—H, —C(═O)-Rw, —C(═O)—O-Rw, —C(═O)—S—Rw, —O—C(═O)—S-Rw, —O—C(═O)-Rw, —O—C(═O)—O-Rw, —S-Rw1, —C(═S)-Rw, —C(═S)—O-Rw, —C(═S)—S-Rw, —O—C(═S)—S-Rw, an alkynyl, an alkenyl and mixtures thereof, where Rw is a C-1 to C-18 substituted or unsubstituted alkyl or an unsubstituted or substituted aryl, Rw1 is a substituted or unsubstituted aryl.

In another aspect of this invention, described herein where said covalent compound has either structures (I), (Ia), (Ib), (Ic), (Id), (Ie) or (If), R₂ is a further substituted phenyl, which when np is 1, 2, 3 4 or 5, is further substituted with only 1 further substituent which selected from —NO₂, —OH, —O-Rw, —CN, —F, —Cl, —Br, —I, —S(═O)₂-Rw, —O—S(═O)₂-Rw, —O—S(═O)₂—O-Rw1, —O—S(═O)-Rw, O—S(═O)—O-Rw1, —C(═O)—H, —C(═O)-Rw, —C(═O)—O-Rw, —C(═O)—S-Rw, —O—C(═O)—S-Rw, —O—C(═O)-Rw, —O—C(═O)—O-Rw, —S-Rw1, —C(═S)-Rw, —C(═S)—O-Rw, —C(═S)—S-Rw, —O—C(═S)—S-Rw, an alkynyl, an alkenyl and mixtures thereof, where Rw is a C-1 to C-18 substituted or unsubstituted alkyl or an unsubstituted or substituted aryl and Rw1 is an unsubstituted or substituted aryl. In one aspect of this embodiment np is 1 and said substituent is located at the 4-position of said further substituted phenyl.

In another aspect of this invention, described herein where said covalent compound has either structures (I), (Ia), (Ib), (Ic), (Id), (Ie) or (If), ), R₂ is a further substituted phenyl, which when np is 1, 2, 3, or 4 is further substituted with only 2 further substituents which are selected from —NO₂, —OH, —O-Rw, —CN, —F, —Cl, —Br, —I, —S(═O)₂-Rw, —O—S(═O)₂-Rw, —O—S(═O)₂—O-Rw1, —O—S(═O)-Rw, —O—S(═O)—O-Rw1, —C(═O)—H, —C(═O)-Rw, —C(═O)—O-Rw, —C(═O)—S-Rw, —O—C(═O)—S-Rw, —O—C(═O)-Rw, —O—C(═O)—O-Rw, —S-Rw1, —C(═S)-Rw, —C(═S)—O-Rw, —C(═S)—S-Rw, —O—C(═S)—S-Rw, an alkynyl, an alkenyl and mixtures thereof, where Rw is a C-1 to C-18 substituted or unsubstituted alkyl or a substituted or unsubstituted aryl and Rw1 is an unsubstituted or substituted aryl. In one aspect of this embodiment, np is 1 and said further substituents are located at the 2-position and 4-position of said substituted phenyl.

In one aspect of this invention, said covalent has structure (Ig), where Rew, Rew₁, Rew₂, Rew₃, and Rew₄, are independently selected from a hydrogen atom, from —NO₂, —OH, —O-Rw, —CN, —F, —Cl, —Br, —I, C-1 to C-18 unsubstituted alkyl, C-1 to C-18 substituted alkyl, —S(═O)₂-Rw, —O—S(═O)₂-Rw, —O—S(═O)₂—O-Rw1, —O—S(═O)-Rw, —O—S(═O)—O-Rw1, —C(═O)—O-Rw, —C(═O)—H, —C(═O)—S-Rw, —O—C(═O)—S-Rw, —O—C(═O)-Rw, —O—C(═O)—O-Rw, —S-Rw1, —C(═S)-Rw, —C(═S)—O-Rw, —C(═S)—S-Rw, —O—C(═S)—S-Rw, an alkynyl, an alkenyl, a naphthaleneimido sulfate moiety of structure (Iga) where ** represents the attachment point of the naphthaleneimido sulfate of structure (Iga), and mixtures thereof, where Rw is a C-1 to C-18 unsubstituted or substituted alkyl, or a substituted or unsubstituted aryl, and Rw1 is an unsubstituted or substituted aryl, and where Rar₅, Rar₆, Rar₇, Rar₈, Rar₉, Rar₁₀, are independently selected from a hydrogen atom, F, Cl, Br, I, a substituted aryl, an unsubstituted aryl, an unsubstituted C-1 to C-18 alkyl a substituted C-1 to C-18 alkyl, an unsubstituted C-1 to C-18 alkoxy, a substituted C-1 to C-18 alkoxy an unsubstituted C-1 to C-18 alkylthio, a substituted C-1 to C-18 alkylthio, and mixtures thereof, where for substituents comprising C-2 to C-18 unsubstituted alkyl, and substituted C-2 to C-18 alkyl these may optionally comprise, within the length of the alkyl moiety, a moiety within their length selected from a carbon to carbon double bond (═), a carbon to carbon triple bond (≡), and a heteroatom comprising moiety selected from —O—, —S—, —C(═O)—, —C(═S)—, —S(═O)₂—, —S(═O)—, —C(═O)—O—, —C(═O)—S—, —O—S(═O)₂—, —O—C(═O)—O—, —C(═O)—NH—, —O—C(═O)—NH—, and —C(═O)—NR—, and mixtures thereof; where R is a C-1 to C-18 alkyl.

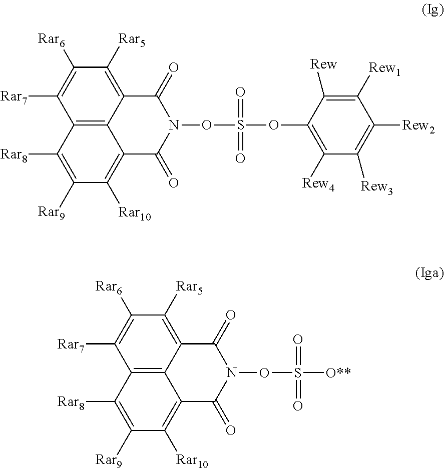

In one aspect said covalent compounds of structure (Ig), it more specifically has structure (Ig-1), R_{1g} is H or a UV sensitizing moiety comprising a chromophore comprising a thio moiety, an oxy (—O—) moiety, a carbonyl moiety, (—C(═O)—), a alkene moiety, an alkyne moiety, and mixtures thereof, and ** represents the attachment point of the naphthaleneimido sulfate of structure (Iga). In one aspect Rw is a C-1 to C-18 unsubstituted or substituted alkyl, or a substituted or unsubstituted aryl. In one aspect of this embodiment Rw is a C-1 to C-8 unsubstituted alkyl, in another it is a C-1 to C-4 unsubstituted alkyl. In another aspect Rw is phenyl. In another aspect Rw1 is phenyl. In one aspect of this embodiment, R_{1g} is H. In another aspect, R_{1g} is said UV sensitizing moiety. In one aspect of these embodiments at least one of Rew, Rew₁, Rew₂, Rew₃ and Rew₄ is selected from an electron withdrawing group selected from —NO₂, —OH, —O-Rw, —CN, —F, —Cl, —Br, —I, —S(═O)₂-Rw, —O—S(═O)₂-Rw, —O—S(═O)₂—O-Rw1, —O—S(═O)-Rw, —O—S(═O)—O-Rw 1, —C(═O)—O-Rw, —C(═O)—H, —C(═O)—S-Rw, —O—C(═O)—S-Rw, —O—C(═O)-Rw, —O—C(═O)—O—Rw, —S-Rw1, —C(═S)-Rw, —C(═S)—O-Rw, —C(═S)—S-Rw, and —O—C(═S)—S-Rw, and naphthaleneimido sulfate moiety of structure (Iga); in one aspect of this embodiment at least two of Rew, Rew₁, Rew₂, Rew₃ and Rew₄ are selected from these electron withdrawing groups; in one aspect of this embodiment at least three of Rew, Rew₁, Rew₂, Rew₃ and Rew₄ are selected from these electron withdrawing groups; in one aspect of this embodiment at least four of Rew, Rew₁, Rew₂, Rew₃ and Rew₄ are selected from these electron withdrawing groups; in one aspect of this embodiment Rew, Rew₁, Rew₂, Rew₃ and Rew₄ are all electron withdrawing groups; in one aspect of this embodiment Rew, Rew₁, Rew₂, Rew₃ and Rew₄ are all the same electron withdrawing group; in one aspect of this embodiment Rew, Rew₁, Rew₂, Rew₃ and Rew₄ are all the same electron withdrawing group and either —Cl, —Br or —F; in one aspect of this embodiment Rew, Rew₁, Rew₂, Rew₃ and Rew₄ are all —Cl; in one aspect of this embodiment Rew, Rew₁, Rew₂, Rew₃ and Rew₄ are all —F.

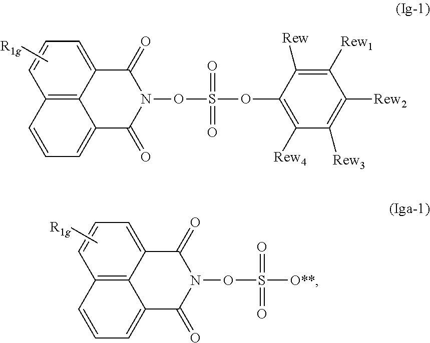

In another aspect of said inventive compound of structure (Ig), it more specifically has structure (Ih). In another aspect of said compound of structure (Ig), it more specifically has structure (Ih-1).

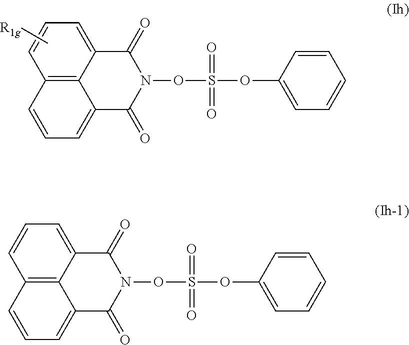

In one aspect of this invention, said covalent compound of structure (Ig) more specifically has structure (Iha) where, Rew₁, and Rew₃, are independently selected from, —OH, —O-Rw, —CN, —F, —Cl, —Br, —I, —S(═O)₂-Rw, —O—S(═O)₂-Rw, —O—S(═O)₂—O-Rw1, —O—S(═O)-Rw, —O—S(═O)—O-Rw1, —C(═O)—H, —C(═O)-Rw, —C(═O)—O-Rw, —C(═O)—S-Rw, —O—C(═O)—S—Rw, —O—C(═O)-Rw, —O—C(═O)—O-Rw, —S-Rw1, —C(═S)-Rw, —C(═S)—O-Rw, —C(═S)—S-Rw, —O—C(═S)—S-Rw, a substituted naphthaleneimido sulfate moiety of structure (Iga), and mixtures thereof, Rw is a C-1 to C-18 unsubstituted or substituted alkyl, or a substituted or unsubstituted aryl, Rw1 is an unsubstituted or substituted aryl, R_{1g} is H or a UV sensitizing moiety comprising a chromophore selected from the groups comprising a thio moiety, an oxy (—O—) moiety, a carbonyl moiety, (—C(═O)—), an alkene moiety, an alkyne moiety, and mixtures thereof, and ** represents the attachment point of the naphthaleneimido sulfate of structure (Iga). In one aspect of this embodiment Rw is a C-1 to C-8 unsubstituted alkyl, in another it is a C-1 to C-4 unsubstituted alkyl. In another aspect Rw is phenyl. In another aspect Rw1 is phenyl. In one aspect of this embodiment, R_{1g} is H and it more specifically has structure (Iha-1), In another, R_{1g} is said UV sensitizing moiety.

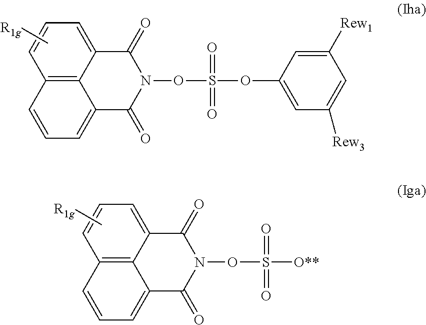

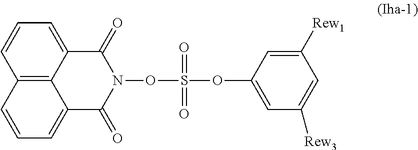

In specific examples of said covalent compound of structure (Iha), it has structure (Iha-1), where Rew₁ and Rew₃ are individually selected from —F, —Cl, —Br, —I, —O-Rw, —O—S(═O)₂-Rw, —O—S(═O)₂—O-Rw1, —O—C(═O)-Rw, O—C(═O)—S-Rw, and —O—C(═O)—O-Rw and a substituted naphthaleneimido sulfate moiety of structure (Iga). In one aspect structure (Iha), Rew₁ and Rew₃ are individually selected from F, Cl, Br, I, in one aspect of this embodiment it may have structures (Iha-2), (Iha-3), or (Iha-4). In one aspect of these embodiment R_{1g} is said UV sensitizing moiety comprising a chromophore, in another aspect R_{1g} is H.

In another aspect of said covalent compound of structure (Iha), Rew₁ and Rew₃, are the same and are selected from —O-Rw, —O—S(═O)₂—O-Rw1, —F, —Cl, —Br, —I, —O-Rw, —O—S(═O)₂-Rw, —O—S(═O)₂—O-Rw1, —O—C(═O)-Rw, O—C(═O)—S-Rw, and —O—C(═O)—O-Rw and a substituted naphthaleneimido sulfate moiety of structure (Iga). In one aspect of this embodiment R_{1g} is H. In another aspect of this embodiment R_{1g} is said UV sensitizing moiety

In another aspect of said covalent compound of structure (Iha-2), it more specifically has structure (Iha-3), in another it more specifically has structure (Iha-4), in another is more specifically has structure (Iha-5), in another it more specifically has structure (Iha-6). In one of the embodiments R_{1g} of these more specific structure R_{1g} is H, in another it is said UV sensitizing moiety comprising a chromophore.

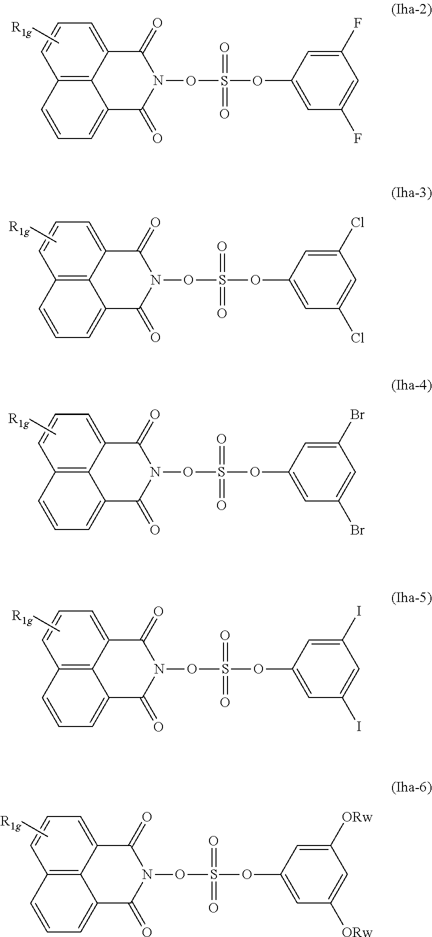

In one aspect of this invention, said covalent compound of structure (Ig) more specifically has structure (Ihb) where Rew, Rew₂, and Rew₄, are independently selected from a hydrogen atom, and the electron withdrawing groups —NO₂, —OH, —O-Rw, —CN, —F, —Cl, —Br, —I, —S(═O)₂-Rw, —O—S(═O)₂-Rw, —O—S(═O)₂—O-R w1, —O—S(═O)-Rw, —O—S(═O)—O-Rw1, —C(═O)—H, —C(═O)-Rw, —C(═O)—O-Rw, —C(═O)—S-Rw, —O—C(═O)—S-Rw, —O—C(═O)-Rw, —O—C(═O)—O-Rw, —S-Rw1, —C(═S)-Rw, —C(═S)—O-Rw, —C(═S)—S-Rw, —O—C(═S)—S-Rw, and a substituted naphthaleneimido sulfate moiety of structure (Iga), and mixtures thereof, where Rw is a C-1 to C-18 unsubstituted or substituted alkyl, or a substituted or unsubstituted aryl, Rw1 is an unsubstituted or substituted aryl, where at least one of Rew, Rew₂, and Rew₄, is not a hydrogen atom, and R_{1g} is H or a UV sensitizing moiety comprising a chromophore selected from the group consisting of a thio moiety, an oxy (—O—) moiety, a carbonyl moiety, (—C(═O)—), a alkene moiety, an alkyne moiety and mixtures thereof, and ** represents the attachment point of the naphthaleneimido sulfate of structure (Iga). In one aspect of this embodiment Rw is a C-1 to C-8 unsubstituted alkyl, in another it is a C-1 to C-4 unsubstituted alkyl. In another aspect Rw is phenyl. In another aspect Rw1 is phenyl. In one aspect of this embodiment, R_{1g} is H and it more specifically has structure (Ihb-1), in another, R_{1g} is said UV sensitizing moiety.

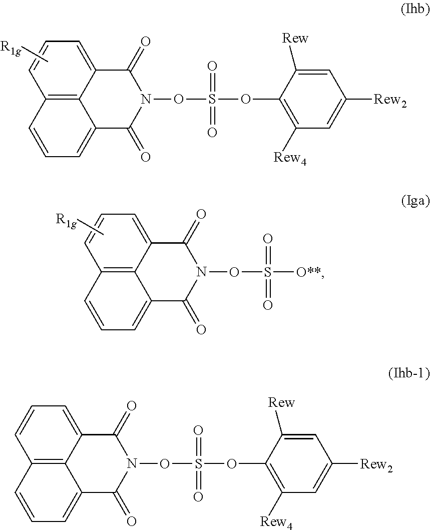

In one aspect of this invention, said covalent compound of structure (Ig) it is one where Rew, Rew₂, and Rew₄, are individually selected from H, and the electron withdrawing groups —NO₂, —CN, —S(═O)₂-Rw, —C(═O)—H, —C(═O)-Rw, —C(═O)—O-Rw, —C(═O)—S-Rw, —C(═S)-Rw, —C(═S)—O-Rw, and —C(═S)—S-Rw, where at least one of Rew, Rew₂, and Rew₄ is not a hydrogen. In another aspect of this embodiment Rew, Rew₂, and Rew₄ none of these is hydrogen, in one aspect of this embodiment Rew, Rew₂, and Rew₄ are the same electron withdrawing group, in another aspect of this embodiment at least two of Rew, Rew₂, and Rew₄ are different electronic withdrawing groups. In one aspect of this embodiment R_{1g} is H. In another aspect R_{1g} is said UV sensitizing moiety.

In one aspect of this invention, said covalent compound has structure (Ii), wherein X is 0 or S, Ri₁ is a C-1 to C-18 substituted or unsubstituted alkyl and mixtures thereof, where for C-2 to C-18 unsubstituted alkyl, and substituted C-2 to C-18 alkyl these may optionally comprise a moiety within their length selected from a carbon to carbon double bond (═), a carbon to carbon triple bond (≡), a heteroatom comprising moiety selected from —O—, —S—, —C(═O)—, —C(═S)—, —S(═O)₂—, —S(═O)—, —C(═O)—O—, —C(═O)—S—, —O—S(═O)₂—, —O—C(═O)—O—, —C(═O)—NH—, —O—C(═O)—NH—, — and —C(═O)—NR—, and mixtures thereof; where R is a C-1 to C-18 alkyl; and where Rew, Rew₁, Rew₂, Rew₃, and Rew₄, are independently selected from a hydrogen atom, —NO₂, —OH, —O-Rw, —CN, —F, —Cl, —Br, —I, a C-1 to C-18 unsubstituted alkyl, a C-1 to C-18 substituted alkyl, phenyl, a substituted phenyl, —S(═O)₂-Rw, —O—S(═O)₂-Rw, —O—S(═O)₂—O-Rw1, —O—S(═O)-Rw, —O—S(═O)—O-Rw1, —C(═O)—H, —C(═O)-Rw, —C(═O)—O-Rw, —C(═O)—S-Rw, —O—C(═O)—S—Rw, —O—C(═O)-Rw, —O—C(═O)—O-Rw, —S-Rw1, —C(═S)-Rw, —C(═S)—O-Rw, —C(═S)—S-Rw, —O—C(═S)—S-Rw, an alkynyl, an alkenyl, a substituted naphthaleneimido sulfate moiety of structure (Ii-1), and mixtures thereof, where Rw is a C-1 to C-18 substituted or unsubstituted alkyl or an unsubstituted or substituted aryl and Rw1 is an unsubstituted or substituted aryl and ** represents the attachment point of the substituted naphthaleneimido sulfate moiety of structure (Ii-1). In one aspect of this embodiment Rw is a C-1 to C-8 unsubstituted alkyl, in another it is a C-1 to C-4 unsubstituted alkyl. In another aspect Rw1 is phenyl. In one aspect of this embodiment it is one where Rew, Rew₁, Rew₂, Rew₃ and Rew₄, are individually selected from H, and the electron withdrawing groups —NO₂, —CN, —S(═O)₂-Rw, —C(═O)—H, —C(═O)-Rw, —C(═O)—O-Rw, —C(═O)—S-Rw, —C(═S)-Rw, —C(═S)—O-Rw, and —C(═S)—S-Rw, where at least one of Rew, Rew₁, Rew₂, Rew₃ and Rew₄ is not a hydrogen.

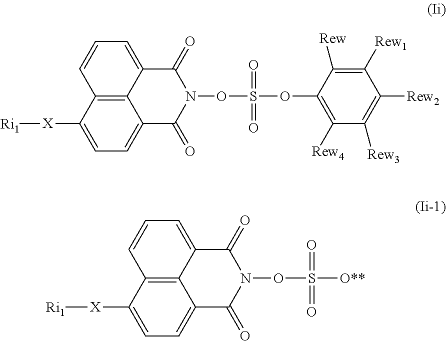

In one aspect of the compound having structure (Ii) it more specifically has structure (Iia), where when Ri₁ is a C-1 to C-18 or C-2 to C-18 substituted or unsubstituted alkyl, and mixtures thereof, where for C-2 to C-18 unsubstituted alkyl, and substituted C-2 to C-18 alkyl these may optionally comprise a moiety within their length selected from a carbon to carbon double bond (═), a carbon to carbon triple bond (≡), and a heteroatom comprising moiety selected from —O—, —S—, —C(═O)—, —C(═S)—, —S(═O)₂—, —S(═O)—, —C(═O)—O—, —C(═O)—S—, —O—S(═O)₂—, —O—C(═O)—O—, —C(═O)—NH—, —O—C(═O)—NH—, and —C(═O)—NR—, and mixtures thereof, where R is a C-1 to C-18 alkyl; and where Rew, Rew₁, Rew₂, Rew₃, and Rew₄, are independently selected from a hydrogen atom, —NO₂, —OH, —O-Rw, —CN, —F, —Cl, —Br, —I, a C-1 to C-18 unsubstituted alkyl, a C-1 to C-18 substituted alkyl, phenyl, a substituted phenyl, —S(═O)₂-Rw, —O—S(═O)₂-Rw, —O—S(═O)₂—O-Rw1, —O—S(═O)-Rw, —O—S(═O)—O—Rw1, —C(═O)—H, —C(═O)-Rw, —C(═O)—O-Rw, —C(═O)—S-Rw, —O—C(═O)-Rw, —O—C(═O)—S—Rw, —O—C(═O)—O-Rw, —S-Rw1, —C(═S)-Rw, —C(═S)—O-Rw, —C(═S)—S-Rw, —O—C(═S)—S-Rw, an alkynyl, an alkenyl, a substituted naphthaleneimido sulfate moiety of structure (Iia-1), and mixtures thereof, where Rw is a C-1 to C-18 substituted or unsubstituted alkyl or an unsubstituted or substituted aryl and Rw1 is an unsubstituted or substituted aryl and ** represents the attachment point of the substituted naphthaleneimido sulfate moiety of structure (Iia-1). In one aspect of this embodiment Rw is a C-1 to C-8 unsubstituted alkyl, in another it is a C-1 to C-4 unsubstituted alkyl. In another aspect Rw1 is phenyl. In one aspect of this embodiment it is one where Rew, Rew₁, Rew₂, Rew₃ and Rew₄, are individually selected from H, and the electron withdrawing groups —NO₂, —CN, —S(═O)₂-Rw, —C(═O)—H, —C(═O)-Rw, —C(═O)—O-Rw, —C(═O)—S-Rw, —C(═S)-Rw, —C(═S)—O-Rw, and —C(═S)—S-Rw, where at least one of Rew, Rew₁, Rew₂, Rew₃ and Rew₄ is not a hydrogen.

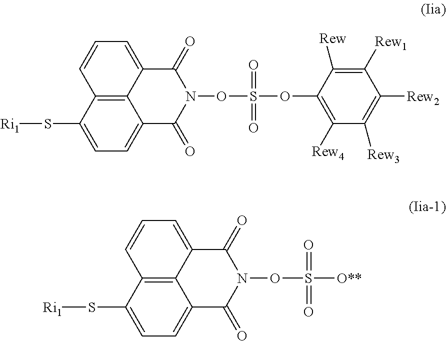

In one aspect of said covalent compound having structure (Iia), Ri₁ is a C-2 to C-18 substituted or unsubstituted alkyl. In another aspect of this embodiment Ri₁ is a C-1 to C-8 substituted or unsubstituted alkyl, in another it is a C-1 to C-4 substituted or unsubstituted alkyl.

In one aspect of this invention, said covalent compound has structure (Iia), where Ri₁ is a C-2 to C-18 an unsubstituted alkyl. In one aspect of this embodiment Ri₁ is a C-2 to C-8 unsubstituted alkyl, in another it is a C-2 to C-4 unsubstituted alkyl.

In one aspect of this invention, said covalent compound has structure (Iia), where Ri₁ is a C-3 to C-18 branched alkyl. In another aspect of this embodiment, it is a C-3 to C-8 branched alkyl, in another it is a C-3 to C-5 branched alkyl. In another aspect of this embodiment, it has structure (Iia-2), where Rew, Rew₁, Rew₂, Rew₃ and Rew₄ are defined as for structure (Iia) and where substituted naphthaleneimido sulfate moiety of structure (Iia-1) has structure (Iia-2a) and ** represents the attachment point of the substituted naphthaleneimido sulfate moiety of structure (Iia-2a). In another aspect of this embodiment, it has structure (Iia-3).

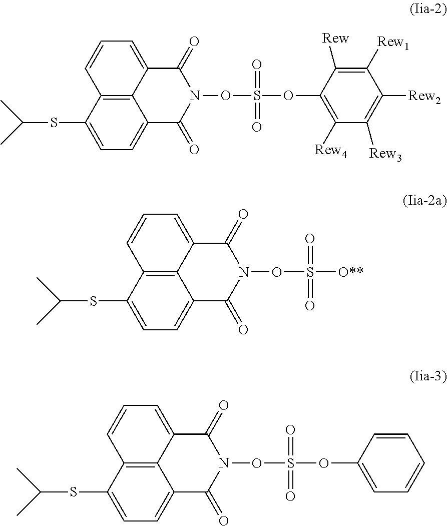

In one aspect of this invention, said covalent compound has structure (Iia′), where Ri₁ is a C-2 to C-18 unsubstituted alkyl, and where, Rew, Rew₁, Rew₂, Rew₃, and Rew₄, are independently selected from the group consisting of a hydrogen atom, —NO₂, —OH, —O-Rw, —CN, —F, —Cl, —Br, —I, a C-1 to C-18 unsubstituted alkyl, a C-1 to C-18 substituted alkyl, phenyl, a substituted phenyl, —S(═O)₂-Rw, —O—S(═O)₂-Rw, —O—S(═O)₂—O-Rw1, —O—S(═O)-Rw, —O—S(═O)—O-Rw1, —C(═O)—H, —C(═O)-Rw, —C(═O )—O-Rw, —C(═O)—S-Rw, —O—C(═O)—S-Rw, —O—C(═O)-Rw, —O—C(═O)—O-Rw, —S-Rw1, —C(═S)-Rw, —C(═S)—O-Rw, —C(═S)—S-Rw, —O—C(═S)—S-Rw, an alkynyl, an alkenyl, a substituted naphthaleneimido sulfate moiety of structure (Iia′-1), and mixtures thereof, where Rw is a C-1 to C-18 substituted or unsubstituted alkyl or an unsubstituted or substituted aryl and Rw1 is an unsubstituted or substituted aryl and ** represents the attachment point of the substituted naphthaleneimido sulfate moiety of structure (Iia-1). In one aspect of these embodiments they are ones where Rew, Rew₁, Rew₂, Rew₃ and Rew₄, are individually selected from H, and the electron withdrawing groups —NO₂, —CN, —S(═O)₂-Rw, —C(═O)—H, —C(═O)-Rw, —C(═O)—O-Rw, —C(═O)—S-Rw, —C(═S)-Rw, —C(═S)—O-Rw, and —C(═S)—S-Rw, were at least one of Rew, Rew₁ Rew₂, Rew₃ and Rew₄ is not a hydrogen.

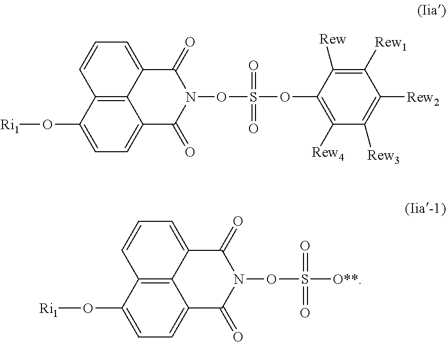

In one aspect of this invention, said covalent compound has structure (Ij), wherein X is 0 or S, Ri₁ is a C-1 to C-18 alkyl substituted or unsubstituted alkyl and mixtures thereof, where for C-2 to C-18 unsubstituted alkyl, and substituted C-2 to C-18 alkyl these may optionally comprise a moiety within their length selected from a carbon to carbon double bond (═), a carbon to carbon triple bond (≡), and a heteroatom comprising moiety selected from —O—, —S—, —C(═O)—, —C(═S)—, —S(═O)₂—, —S(═O)—, —C(═O)—O—, —C(═O)—S—, —O—S(═O)₂—, —O—C(═O)—O—, —C(═O)—NH—, —O—C(═O)—NH—, and —C(═O)—NR—, and mixtures thereof, where R is a C-1 to C-18 alkyl, and where Rew, Rew₁, Rew₂, Rew₃, and Rew₄, are independently selected from a hydrogen atom, —NO₂, —OH, —O-Rw, —CN, —F, —Cl, —Br, —I, a C-1 to C-18 unsubstituted alkyl, a C-1 to C-18 substituted alkyl, phenyl, a substituted phenyl, —S(═O)₂-Rw, —O—S(═O)₂-Rw, —O—S(═O)₂—O-Rw1, —O—S(═O)-Rw, —O—S(═O)—O-Rw 1, —C(═O)—H, —C(═O)-Rw, —C(═O)—O-Rw, —C(═O)—S-Rw, —O—C(═O)—S—Rw, —O—C(═O)-Rw, —O—C(═O)—O-Rw, —S-Rw1, —C(═S)-Rw, —C(═S)—O-Rw, —C(═S)—S-Rw, —O—C(═S)—S-Rw, an alkynyl, an alkenyl, a substituted naphthaleneimido sulfate moiety of structure (Ij-1), and mixtures thereof, where Rw is a C-1 to C-18 substituted or unsubstituted alkyl or an unsubstituted or substituted aryl and Rw1 is an unsubstituted or substituted aryl and ** represents the attachment point of the substituted naphthaleneimido sulfate moiety of structure (Ij-1). In one aspect of this embodiment Rw is a C-1 to C-8 unsubstituted alkyl, in another it is a C-1 to C-4 unsubstituted alkyl. In another aspect Rw is phenyl. In another aspect Rw1 is phenyl. In one aspect of these embodiments they are ones where Rew, Rew₁, Rew₂, Rew₃ and Rew₄, are individually selected from H, and the electron withdrawing groups —NO₂, —CN, —S(═O)₂-Rw, —C(═O)—H, —C(═O)-Rw, —C(═O)—O-Rw, —C(═O)—S-Rw, —C(═S)-Rw, —C(═S)—O-Rw, and —C(═S)—S-Rw, were at least one of Rew, Rew₁, Rew₂, Rew₃ and Rew₄ is not a hydrogen.

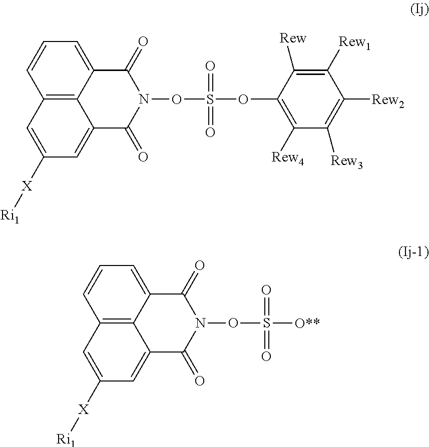

In one aspect of the compound having structure (Ij) it more specifically has structure (Ija), where Ri₁ is a C-1 to C-18 substituted or unsubstituted alkyl, and mixtures thereof, where for C-2 to C-18 unsubstituted alkyl, and substituted C-2 to C-18 alkyl these may optionally comprise a moiety within their length selected from a carbon to carbon double bond (═), a carbon to carbon triple bond (≡), and a heteroatom comprising moiety selected from —O—, —S—, —C(═O)—, —C(═S)—, —S(═O)₂—, —S(═O)—, —C(═O)—O—, —C(═O)—S—, —O—S(═O)₂—, —O—C(═O)—O—, —C(═O)—NH—, —O—C(═O)—NH—, and —C(═O)—NR—, and mixtures thereof, where R is a C-1 to C-18 alkyl; where Rew, Rew₁, Rew₂, Rew₃, and Rew₄, are independently selected from a hydrogen atom, —NO₂, —OH, —O-Rw, —CN, —F, —Cl, —Br, —I, a C-1 to C-18 unsubstituted alkyl, a C-1 to C-18 substituted alkyl, phenyl, a substituted phenyl, —S(═O)₂-Rw, —O—S(═O)₂-Rw, —O—S(═O)₂—O-Rw1, —O—S(═O)-Rw, —O—S(═O)—O—Rw1, —C(═O)—H, —C(═O)-Rw, —C(═O)—O-Rw, —C(═O)—S-Rw, —O—C(═O)—S—Rw, —O—C(═O)-Rw, —O—C(═O)—O-Rw, —S-Rw1, —C(═S)-Rw, —C(═S)—O-Rw, —C(═S)—S-Rw, —O—C(═S)—S-Rw, an alkynyl, an alkenyl, a substituted naphthaleneimido sulfate moiety of structure (Ija-1), and mixtures thereof, where Rw is a C-1 to C-18 substituted or unsubstituted alkyl or an unsubstituted or substituted aryl and Rw1 is a substituted or unsubstituted aryl and ** represents the attachment point of the substituted naphthaleneimido sulfate moiety of structure (Ija-1). In one aspect of this embodiment Rw is a C-1 to C-8 unsubstituted alkyl, in another it is a C-1 to C-4 unsubstituted alkyl. In another aspect Rw is phenyl. In another aspect Rw1 is phenyl.

In one aspect of said covalent compound has structure (Ija), Ri₁ is a C-2 to C-18 substituted or unsubstituted alkyl. In another aspect of this embodiment Ri₁ is a C-1 to C-8 substituted or unsubstituted alkyl, in another it is a C-1 to C-4 substituted or unsubstituted alkyl.

In one aspect of this invention, said covalent compound has structure (Ija), where Ri₁ is a C-2 to C-18 unsubstituted alkyl. In one aspect of this embodiment Ri₁ is a C-2 to C-8 unsubstituted alkyl, in another it is a C-2 to C-4 unsubstituted alkyl.

In one aspect of this invention, said covalent compound has structure (Ija), where Ri₁ is a C-3 to C-18 branched alkyl. In another aspect of this embodiment, it is a C-3 to C-8 branched alkyl, in another it is a C-3 to C-5 branched alkyl. In another aspect of this embodiment, it has structure (Ija-2), where Rew, Rew₁, Rew₂, Rew₃ and Rew₄ are defined as for structure (Ija) and where substituted naphthaleneimido sulfate moiety of structure (Ija-1) has structure (Ija-2a) and ** represents the attachment point of the substituted naphthaleneimido sulfate moiety of structure (Ija-2a). In another aspect of this embodiment, it has structure (Ija-3). In one aspect of these embodiments they are ones where Rew, Rew₁, Rew₂, Rew₃ and Rew₄, are individually selected from H, and the electron withdrawing groups —NO₂, —CN, —S(═O)₂-Rw, —C(═O)—H, —C(═O)-Rw, —C(═O)—O-Rw, —C(═O)—S-Rw, —C(═S)-Rw, —C(═S)—O-Rw, and —C(═S)—S-Rw, were at least one of Rew, Rew₁, Rew₂, Rew₃ and Rew₄ is not a hydrogen.

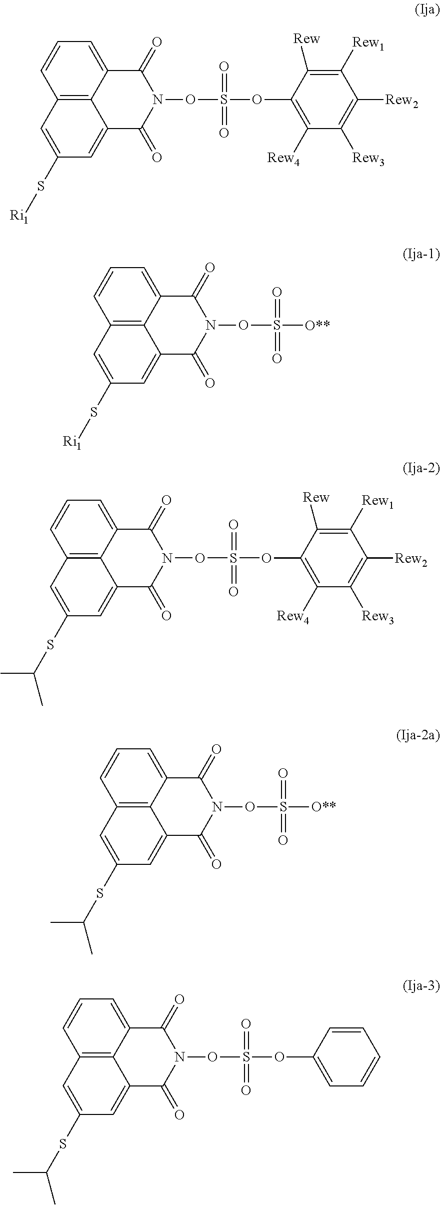

In one aspect of the compound having structure (Ij) it more specifically has structure (Ija′), where R₁₁ is a C-1 to C-18 substituted or unsubstituted alkyl, and mixtures thereof, where for C-2 to C-18 unsubstituted alkyl, and substituted C-2 to C-18 alkyl these may optionally comprise a moiety within their length selected from a carbon to carbon double bond (═), a carbon to carbon triple bond (≡), and a heteroatom comprising moiety selected from —O—, —S—, —C(═O)—, —C(═S)—, —S(═O)₂—, —S(═O)—, —C(═O)—O—, —C(═O)—S—, —O—S(═O)₂—, —O—C(═O)—O—, —C(═O)—NH—, —O—C(═O)—NH—, and —C(═O)—NR—, and mixtures thereof, where Rew, Rew₁, Rew₂, Rew₃, and Rew₄, are independently selected from a hydrogen atom, —NO₂, —OH, —O-Rw, —CN, —F, —Cl, —Br, —I, a C-1 to C-18 unsubstituted alkyl, a C-1 to C-18 substituted alkyl, phenyl, a substituted phenyl, —S(═O)₂-Rw, —O—S(═O)₂-Rw, —O—S(═O)₂—O-Rw1, —O—S(═O)-Rw, —O—S(═O)—O—Rw1, —C(═O)—H, —C(═O)-Rw, —C(═O)—O-Rw, —C(═O)—S-Rw, —O—S(═O)₂-Rw, —O—C(═O)—S—Rw, —O—C(═O)-Rw, —O—C(═O)—O-Rw, —S-Rw1, —C(═S)-Rw, —C(═S)—O-Rw, —C(═S)—S-Rw, —O—C(═S)—S-Rw, an alkynyl, an alkenyl, a substituted naphthaleneimido sulfate moiety of structure (Ija′-1), and mixtures thereof, where Rw is a C-1 to C-18 substituted or unsubstituted alkyl or an unsubstituted or substituted aryl and Rw1 is a substituted or unsubstituted aryl and ** represents the attachment point of the substituted naphthaleneimido sulfate moiety of structure (Ija′-1). In one aspect of this embodiment Rw is a C-1 to C-8 unsubstituted alkyl, in another it is a C-1 to C-4 unsubstituted alkyl. In another aspect Rw is phenyl. In another aspect Rw1 is phenyl. In one aspect of these embodiments they are ones where Rew, Rew₁ Rew₂, Rew₃ and Rew₄, are individually selected from H, and the electron withdrawing groups —NO₂, —CN, —S(═O)₂-Rw, —C(═O)—H, —C(═O)-Rw, —C(═O)—O-Rw, —C(═O)—S-Rw, —C(═S)-Rw, —C(═S)—O-Rw, and —C(═S)—S-Rw, were at least one of Rew, Rew₁, Rew₂, Rew₃ and Rew₄ is not a hydrogen.

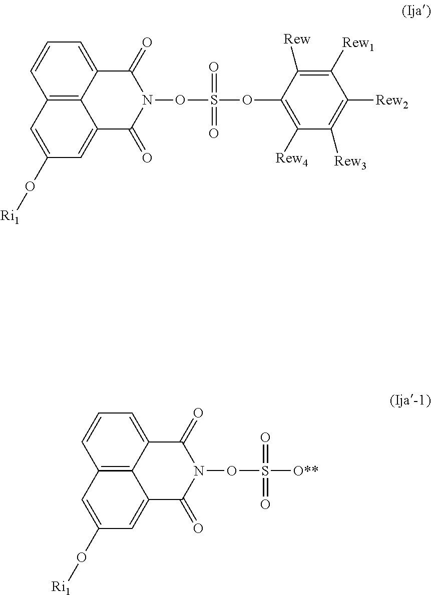

In one aspect of this invention, said covalent compound has structure (Ik), wherein X₁ is direct valence bond, or a C-1 to C-18 alkylene spacer (e.g. a C-1 to C-8 alkylene spacer), Ro and Rol are independently selected from a hydrogen atom, a C-1 to C-18 unsubstituted alkyl, a substituted C-1 to C-18 alkyl and mixtures thereof, where for C-2 to C-18 unsubstituted alkyl, and substituted C-2 to C-18 alkyl these may optionally comprise a moiety within their length selected from a carbon to carbon double bond (═), a carbon to carbon triple bond (≡), and a heteroatom comprising moiety selected from —O—, —S—, —C(═O)—, —C(═S)—, —S(═O)₂—, —S(═O)—, —C(═O)—O—, —C(═O)—S—, —O—S(═O)₂—, —O—C(═O)—O—, —C(═O)—NH—, —O—C(═O)—NH—, and —C(═O)—NR—, and mixtures thereof; and where R is a C-1 to C-18 alkyl; and where Rew, Rew₁, Rew₂, Rew₃, and Rew₄, are independently selected from hydrogen, —NO₂, —OH, —O-Rw, —CN, —F, —Cl, —Br, —I, a C-1 to C-18 unsubstituted alky 1, a C-1 to C-18 substituted alkyl, phenyl, a substituted phenyl, —S(═O)₂-Rw, —O—S(═O)₂-Rw, —O—S(═O)₂—O-Rw1, —O—S(═O)-Rw, —O—S(═O)—O-Rw1, —C(═O)—H, —C(═O)-Rw, —C(═O)—O-Rw, —C(═O)—S-Rw, —O—C(═O)—S-Rw, —O—C(═O)-Rw, —O—C(═O)—O-Rw, —S-Rw1, —C(═S)-Rw, —C(═S)—O-Rw, —C(═S)—S-Rw, —O—C(═S)—S-Rw, an alkynyl, an alkenyl, a substituted naphthaleneimido sulfate moiety of structure (Ik-1), and mixtures thereof, where Rw is a C-1 to C-18 substituted or unsubstituted alkyl or an unsubstituted or substituted aryl and Rw1 is an unsubstituted or substituted aryl and ** represents the attachment point of the substituted naphthaleneimido sulfate moiety of structure (Ik-1). In one aspect of this embodiment Rw is a C-1 to C-8 unsubstituted alkyl, in another it is a C-1 to C-4 unsubstituted alkyl. In another aspect Rw is phenyl. In another aspect Rw1 is phenyl.

In another aspect of this embodiment both Ro and Roi are both a hydrogen atom. In another aspect of this embodiment at least one of Ro and Roi is an unsubstituted C-1 to C-18 alkyl, in another aspect of this embodiment this is a C-1 to C-8 unsubstituted alkyl in another it is a C-1 to C-4 unsubstituted alkyl. In another aspect of this embodiment at least one of Ro and Roi is a substituted C-1 to C-18 alkyl, in another aspect of this embodiment this is a C-1 to C-8 substituted alkyl in another it is a C-1 to C-4 substituted alkyl. In one aspect of these embodiments they are ones were Rew, Rew₁, Rew₂, Rew₃ and Rew₄, are individually selected from H, and the electron withdrawing groups —NO₂, —CN, —S(═O)₂-Rw, —C(═O)—H, —C(═O)-Rw, —C(═O)—O-Rw, —C(═O)—S-Rw, —C(═S)-Rw, —C(═S)—O-Rw, and —C(═S)—S-Rw, were at least one of Rew, Rew₁, Rew₂, Rew₃ and Rew₄ is not a hydrogen.

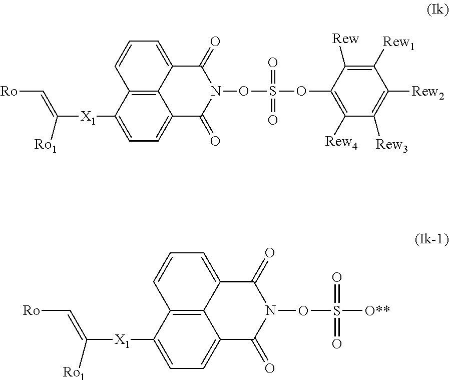

In one aspect of this invention, said covalent compound has structure (Il), wherein X₁ is direct valence bond, or a C-1 to C-18 alkylene spacer (e.g. a C-1 to C-8 alkylene spacer), Ro and Rol are independently selected from a hydrogen atom, a C-1 to C-18 unsubstituted alkyl, a substituted C-1 to C-18 alkyl, and mixtures thereof, where said C-2 to C-18 unsubstituted alkyl and substituted C-2 to C-18 alkyl, are ones either comprising or not a moiety within their length independently selected from a carbon to carbon double bond (═), a carbon to carbon triple bond (—), a heteroatom comprising moiety selected from —O—, —S—, —C(═O)—, —C(═S)—, —S(═O)₂—, —S(═O)—, —C(═O)—O—, —C(═O)—S—, —O—S(═O)₂—, —O—C(═O)—O—, —C(═O)—NH—, —O—C(═O)—NH—, and —C(═O)—NR—, where R is a C-1 to C-18 alkyl, and mixtures thereof; and where Rew, Rew₁, Rew₂, Rew₃, and Rew₄, are independently selected of a hydrogen atom, —NO₂, —OH, —O-Rw, —CN, —F, —Cl, —Br, —I, a C-1 to C-18 unsubstituted alkyl, a C-1 to C-18 substituted alkyl, phenyl, a substitute d phenyl, —S(═O)₂-Rw, —O—S(═O)₂-Rw, —O—S(═O)₂—O-Rw1, —O—S(═O)-Rw, —O—S(═O)—O—Rw1, —C(═O)—H, —C(═O)-Rw, —C(═O)—O-Rw, —C(═O)—S-Rw, —O—C(═O)—S—Rw, —O—C(═O)-Rw, —O—C(═O)—O-Rw, —S-Rw1, —C(═S)-Rw, —C(═S)—O-Rw, —C(═S)—S-Rw, —O—C(═S)—S-Rw, an alkynyl, an alkenyl, a substituted naphthaleneimido sulfate moiety of structure (Il-1), and mixtures thereof, where Rw is a C-1 to C-18 substituted or unsubstituted alkyl or an unsubstituted or substituted aryl and Rw1 is an unsubstituted or substituted aryl. In one aspect of this embodiment Rw is a C-1 to C-18 substituted or unsubstituted alkyl or a substituted or unsubstituted aryl and Rw1 is an unsubstituted or substituted aryl and ** represents the attachment point of the substituted naphthaleneimido sulfate moiety of structure (Il-1). In another Rw is a C-1 to C-4 unsubstituted alkyl. In another aspect Rw is phenyl. In another aspect, Rw1 is phenyl. In another aspect of this embodiment both Ro and Roi are both a hydrogen atom. In another aspect of this embodiment at least one of Ro and Roi is an unsubstituted C-1 to C-18 alkyl, in another aspect of this embodiment this is a C-1 to C-8 unsubstituted alkyl in another it is a C-1 to C-4 unsubstituted alkyl. In another aspect of this embodiment at least one of Ro and Roi is a substituted C-1 to C-18 alkyl, in another aspect of this embodiment this is a C-1 to C-8 substituted alkyl in another it is a C-1 to C-4 substituted alkyl. In one aspect of these embodiments they are ones where Rew, Rew₁, Rew₂, Rew₃ and Rew₄, are individually selected from H, and the electron withdrawing groups —NO₂, —CN, —S(═O)₂-Rw, —C(═O)—H, —C(═O)-Rw, —C(═O)—O-Rw, —C(═O)—S-Rw, —C(═S)-Rw, —C(═S)—O-Rw, and —C(═S)—S-Rw, were at least one of Rew, Rew₁, Rew₂, Rew₃ and Rew₄ is not a hydrogen.

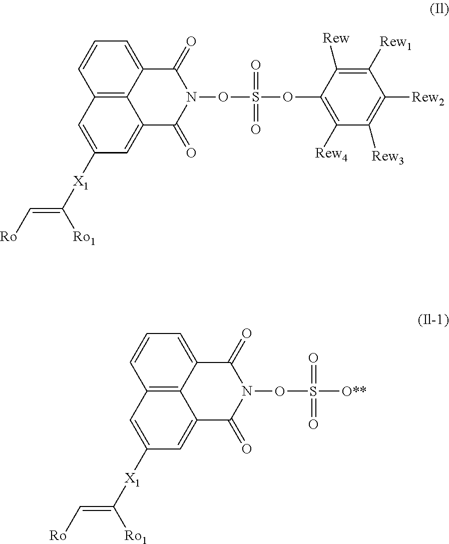

In one aspect of this invention, said covalent compound has structure (Im), wherein X₁ is direct valence bond, or a C-1 to C-8 alkylene spacer, Ro₃ is selected from a hydrogen atom, a phenyl, a substituted phenyl, a thiophen-3-yl, a C-1 to C-18 unsubstituted alkyl, a substituted C-1 to C-18 alkyl, and mixtures thereof, where for C-2 to C-18 unsubstituted alkyl, and substituted C-2 to C-18 alkyl these may optionally comprise a moiety within their length selected from a carbon to carbon double bond (═), a carbon to carbon triple bond (≡), and a heteroatom comprising moiety selected from —O—, —S—, —C(═O)—, —C(═S)—, —S(═O)₂—, —S(═O)—, —C(═O)—O—, —C(═O)—S—, —O—S(═O)₂—, —O—C(═O)—O—, —C(═O)—NH—, —O—C(═O)—NH—, and —C(═O)—NR—, and mixtures thereof, where R is a C-1 to C-18 alkyl; and where Rew, Rew₁, Rew₂, Rew₃, and Rew₄, are independently selected from a hydrogen atom, —NO₂, —OH, —O-Rw, —CN, —F, —Cl, —Br, —I, a C-1 to C-18 unsubstituted alkyl, a C-1 to C-18 substituted alkyl, phenyl, a substituted phenyl, —S(═O)₂-Rw, —O—S(═O)₂-Rw, —O—S(═O)₂—O-Rw1, —O—S(═O)-Rw, —O—S(═O)—O-Rw1, —C(═O)—H, —C(═O)-Rw, —C(═O)—O-Rw, —C(═O)—S-Rw, —O—C(═O)—S—Rw, —O—C(═O)-Rw, —O—C(═O)—O-Rw, —S-Rw1, —C(═S)-Rw, —C(═S)—O-Rw, —C(═S)—S-Rw, —O—C(═S)—S-Rw, an alkynyl, an alkenyl, a substituted naphthaleneimido sulfate moiety of structure (Imn-1), and mixtures thereof, where Rw is a C-1 to C-18 substituted or unsubstituted alkyl or an unsubstituted or substituted aryl and Rw1 is an unsubstituted or substituted aryl and ** represents the attachment point of the substituted naphthaleneimido sulfate moiety of structure (Im-1). In one aspect of this embodiment Rw is a C-1 to C-8 unsubstituted alkyl, in another it is a C-1 to C-4 unsubstituted alkyl. In another aspect Rw is phenyl. In another aspect Rw1 s phenyl.

In one aspect of this embodiment Ro₃ is a hydrogen atom. In another aspect of this embodiment Ro₃ is a C-1 to C-18 substituted or unsubstituted alkyl, in another aspect of this embodiment it is a C-1 to C-8 unsubstituted alkyl and in another aspect of this embodiment it is a C-1 to C-4 unsubstituted alkyl. In another aspect of this embodiment Ro₃ is a C-1 to C-18 substituted alkyl, in another aspect of this embodiment it is a C-1 to C-8 substituted alkyl and in another aspect of this embodiment it is a C-1 to C-4 substituted alkyl. In another aspect of this invention, said covalent compound has structure (Ima), where Ro₃ is selected from phenyl, 4-methoxyphenyl, 4-phenoxyphenyl, and thiophen-3-yl. In one aspect of these embodiments they are ones where Rew, Rew₁, Rew₂, Rew₃ and Rew₄, are individually selected from H, and the electron withdrawing groups —NO₂, —CN, —S(═O)₂-Rw, —C(═O)—H, —C(═O)-Rw, —C(═O)—O-Rw, —C(═O)—S-Rw, —C(═S)-Rw, —C(═S)—O-Rw, and —C(═S)—S-Rw, were at least one of Rew, Rew₁, Rew₂, Rew₃ Rew₃, and Rew₄ is not a hydrogen.

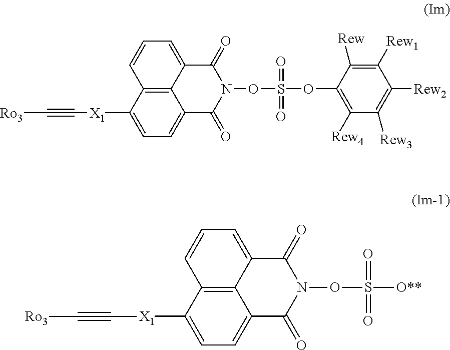

In one aspect of this invention, said covalent compound has structure (Ima), where Ro₃ is selected from phenyl, 4-methoxyphenyl, 4-phenoxyphenyl, and thiophen-3-yl. In another aspect of this invention, said covalent compound has structure (Imb). In another aspect of this embodiment, it has structure (Imc). In another aspect of this embodiment, it has structure (Imd) where Ralk is a C-1 to C-18 alkyl. In each of the embodiments Rew, Rew₁, Rew₂, Rew₃, and Rew₄, may be independently selected a hydrogen atom, —NO₂, —OH, —O-Rw, —CN, —F, —Cl, —Br, —I, a C-1 to C-18 unsubstituted alkyl, a C-1 to C-18 substituted alkyl, phenyl, a substituted phenyl, —S(═O)₂-Rw, —O—S(═O)₂-Rw, —O—S(═O)₂—O-Rw1, —O—S(═O)-Rw, —O—S (═O)—O-Rw1, —C(═O)—H, —C(═O)-Rw, —C(═O)—O-Rw, —C(═O)—S-Rw, —O—C(═O)—S-Rw, —O—C(═O)-Rw, —O—C(═O)—O-Rw, —S-Rw1, —C(═S)-Rw, —C(═S)—O-Rw, —C(═S)—S-Rw, —O—C(═S)—S-Rw, an alkynyl, an alkenyl, and a substituted naphthaleneimido sulfate moiety of structure (Ima-1), where Rw is a C-1 to C-18 substituted or unsubstituted alkyl or an unsubstituted or substituted aryl and Rw1 and ** represents the attachment point of the substituted naphthaleneimido sulfate moiety of structure (Ima-1). In one aspect of these embodiments they are ones where Rew, Rew₂, Rew₃ and Rew₄, are individually selected from H, and the electron withdrawing groups —NO₂, —CN, —S(═O)₂-Rw, —C(═O)—H, —C(═O)-Rw, —C(═O)—O-Rw, —C(═O)—S-Rw, —C(═S)-Rw, —C(═S)—O-Rw, and —C(═S)—S-Rw, were at least one of Rew, Rew₂, Rew₃ and Rew₄ is not a hydrogen.

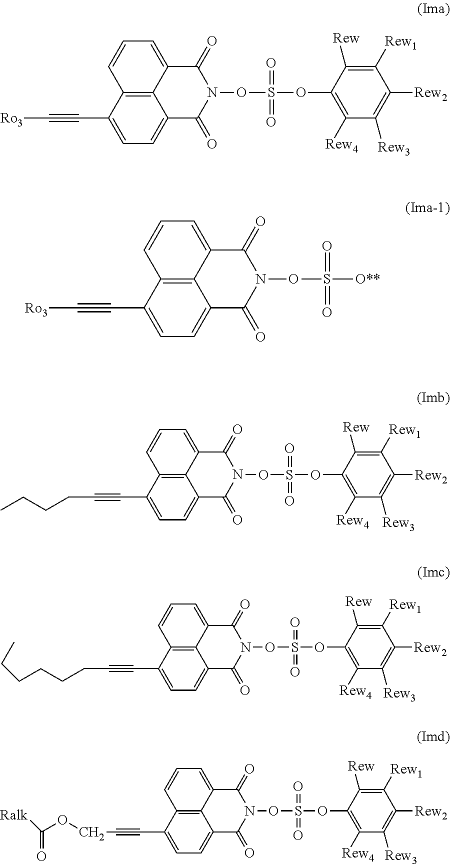

In one aspect of this invention, said covalent compound has structure (In), wherein X₁ is direct valence bond, or a C-1 to C-8 alkylene spacer, Ro₃ is selected from a hydrogen atom, a phenyl, a substituted phenyl, a thiophen-3-yl, a C-1 to C-18 unsubstituted alkyl, a substituted C-1 to C-18 alkyl, and mixtures thereof, where for C-2 to C-18 unsubstituted alkyl, and substituted C-2 to C-18 alkyl these may optionally comprise a moiety within their length selected from a carbon to carbon double bond (═), a carbon to carbon triple bond (≡), and a heteroatom comprising moiety selected from —O—, —S—, —C(═O)—, —C(═S)—, —S(═O)₂—, —S(═O)—, —C(═O)—O—, —C(═O)—S—, —O—S(═O)₂—, —O—C(═O)—O—, —C(═O)—NH—, —O—C(═O)—NH—, and —C(═O)—NR—, and mixtures thereof, where R is a C-1 to C-18 alkyl; and where Rew, Rew₁, Rew₂, Rew₃, and Rew₄, are independently selected from a hydrogen atom, —NO₂, —OH, —O-Rw, —CN, —F, —Cl, —Br, —I, a C-1 to C-18 unsubstituted alkyl, a C-1 to C-18 substituted alkyl, phenyl, a substituted phenyl, —S(═O)₂-Rw, —O—S(═O)₂-Rw, —O—S(═O)₂—O-Rw1, —O—S(═O)-Rw, —O—S(═O)—O-Rw1, —C(═O)—H, —C(═O)-Rw, —C(═O)—O-Rw, —C(═O)—S-Rw, —O—C(═O)—S—Rw, —O—C(═O)-Rw, —O—C(═O)—O-Rw-S-Rw1, —C(═S)-Rw, —C(═S)—O-Rw, —C(═S)—S-Rw, —O—C(═S)—S-Rw, an alkynyl, an alkenyl, a substituted naphthaleneimido sulfate moiety of structure (In-1), and mixtures thereof, where Rw is a C-1 to C-18 substituted or unsubstituted alkyl or an unsubstituted or substituted aryl and Rw1 is an unsubstituted or substituted aryl and ** represents the attachment point of the substituted naphthaleneimido sulfate moiety of structure (In-1). In one aspect of this embodiment Rw is a C-1 to C-8 unsubstituted alkyl, in another it is a C-1 to C-4 unsubstituted alkyl. In another aspect Rw is phenyl. In another aspect Rw1 is phenyl. In one aspect of these embodiments they are ones where Rew, Rew₁, Rew₂, Rew₃ and Rew₄, are individually selected from H, and the electron withdrawing groups —NO₂, —CN, —S(═O)₂-Rw, —C(═O)—H, —C(═O)-Rw, —C(═O)—O-Rw, —C(═O)—S-Rw, —C(═S)-Rw, —C(═S)—O-Rw, and —C(═S)—S-Rw, were at least one of Rew, Rew₁, Rew₂, Rew₃ and Rew₄ is not a hydrogen.

In one aspect of this embodiment, it has structure (Ina), where Ro₃ is selected from phenyl, 4-methoxyphenyl, 4-phenoxyphenyl, and thiophen-3-yl. In another aspect of this embodiment, it has structure (Inb). In another aspect of this embodiment, it has structure (Ina), where Ro₃ is selected from phenyl, 4-methoxyphenyl and 4-phenoxyphenyl. In one aspect of this invention, said covalent has structure has structure (Ina), where Ro₃ is a C-1 to C-18 alkyl. In another aspect of this invention, where said covalent compound has structure (Ina) it has structure (Inb). In another aspect of this invention, where said covalent compound has structure (Ina) it has structure (Inc). In another aspect of this invention, where said covalent compound has structure (Ina), it has structure (Ind) where Ralk is a C-1 to C-18 alkyl. In one aspect of these embodiments they are ones where Rew, Rew₁, Rew₂, Rew₃ and Rew₄, are individually selected from H, and the electron withdrawing groups —NO₂, —CN, —S(═O)₂-Rw, —C(═O)—H, —C(═O)-Rw, —C(═O)—O-Rw, —C(═O)—S-Rw, —C(═S)-Rw, —C(═S)—O-Rw, and —C(═S)—S-Rw, were at least one of Rew, Rew₁, Rew₂, Rew₃ and Rew₄ is not a hydrogen.

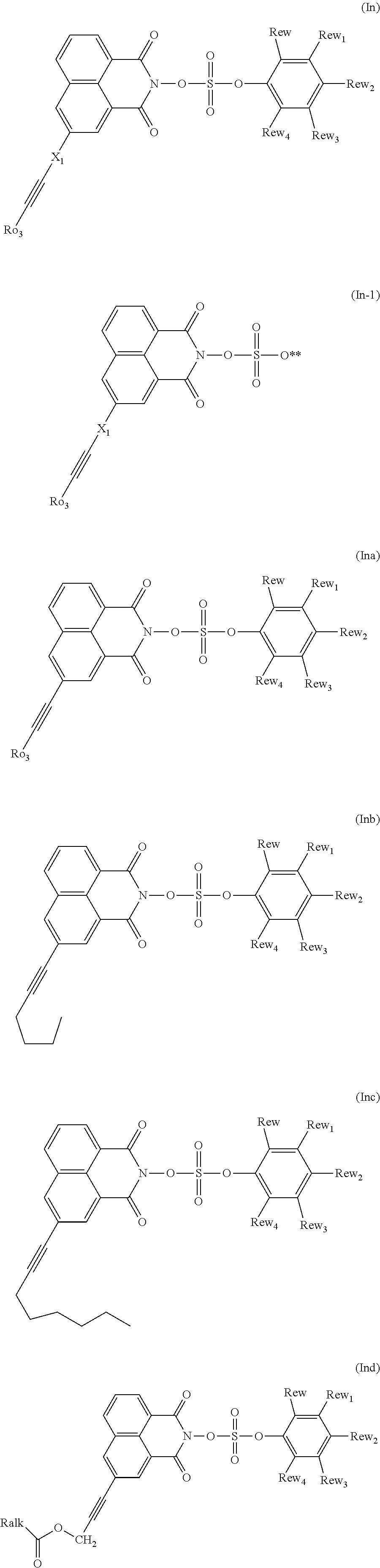

In one embodiment of this invention, these new PAGs have structure (Io), wherein Rnc₁ and Rnc₂ are organic substituents which are free of any fluoroalkyls, any perfluoroalkyls or mixtures of fluoroalkyls and perfluoroalkyls, which are independently selected from an unsubstituted C-1 to C-18 alkyl, a substituted C-1 to C-18 alkyl, an unsubstituted aryl, a substituted aryl and a mixture of these, and R₂ is an unsubstituted aryl or a substituted aryl which is also free of any fluoroalkyls, any perfluoroalkyls or mixtures of fluoroalkyls and perfluoroalkyls and np is 1, 2, 3, 4 or 5. In one aspect of this embodiment both Rnc₁ and Rnc₂ are independently selected from a unsubstituted C-1 to C-18 alkyl, in another aspect both are the same unsubstituted alkyl C-1 to C-18 alkyl; in one aspect of these embodiments Rnc₁ and Rnc₂ are selected from unsubstituted C-1 to C-4 alkyl. In another aspect of this embodiment both Rnc₁ and Rnc₂ are independently selected from an unsubstituted aryl in another aspect both Rnc₁ and Rnc₂ are the same unsubstituted aryl; in one aspect of these embodiments said unsubstituted aryl are selected from phenyl, naphthyl and anthracenyl. In another aspect of this embodiment it has structure (Ioa), where Rew, Rew₁, Rew₂, Rew₃, and Rew₄, are independently selected from a hydrogen atom, —NO₂, —OH, —O-Rw, —CN, —F, —Cl, —Br, —I, a C-1 to C-18 unsubstituted alkyl, a C-1 to C-18 substituted alkyl, phenyl, a substituted phenyl, —S(═O)₂-Rw, —O—S(═O)₂-Rw, —O—S(═O)₂—O-Rw1, —O—S(═O)-Rw, —O—S(═O)—O—Rw1, —C(═O)—H, —C(═O)-Rw, —C(═O)—O-Rw, —C(═O)—S-Rw, —O—C(═O)-Rw, —O—C(═O)—S—Rw, —O—C(═O)—O-Rw, —S-Rw1, —C(═S)-Rw, —C(═S)—O-Rw, —C(═S)—S-Rw, —O—C(═S)—S-Rw, an alkynyl, an alkenyl, a substituted imido sulfate moiety of structure (Ioa-1), and mixtures thereof, where Rw is a C-1 to C-18 substituted or unsubstituted alkyl or an unsubstituted or substituted aryl and Rw1 is an unsubstituted or substituted aryl and ** represents the attachment point of the imido sulfate moiety of structure (Ioa-1). In one aspect of this embodiment Rw is a C-1 to C-8 unsubstituted alkyl, in another it is a C-1 to C-4 unsubstituted alkyl. In another aspect Rw is phenyl. In another aspect Rw1 is phenyl. In one aspect of these embodiments they are ones where Rew, Rew₁, Rew₂ Rew₃, and Rew₄, are individually selected from H, and the electron withdrawing groups —NO₂, —CN, —S(═O)₂-Rw, —C(═O)—H, —C(═O)-Rw, —C(═O)—O-Rw, —C(═O)—S-Rw, —C(═S)-Rw, —C(═S)—O-Rw, and —C(═S)—S-Rw, were at least one of Rew, Rew₁, Rew₂, Rew₃ and Rew₄ is not a hydrogen.

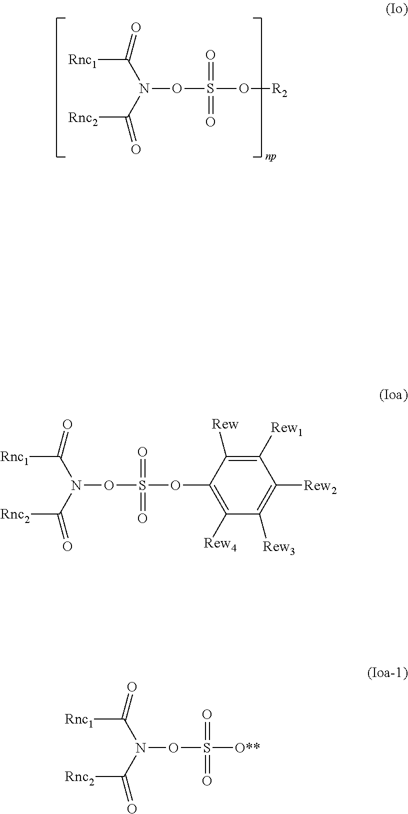

In the compounds of structure (Ia) to (Io), and their substructures described herein, which have a phenyl ring substituted by Rew, Rew₁, Rew₂, Rew₃, and Rew₄, as noted above, particular choices of these will favor increased acidity of the photoacid released after irradiation; in particular ones where at least one of these substituents is an electron withdrawing substituent selected from —NO₂, —OH, —O-Rw, —CN, —F, —Cl, —Br, —I, —S(═O)₂-Rw, —O—S(═O)₂-Rw, —O—S(═O)₂—O-Rw1, —O—S(═O)-Rw, —O—S(═O)—O-Rw1, —C(═O)—H, —C(═O)-Rw, —C(═O)—O-Rw, —C(═O)—S-Rw, —O—C(═O)—S-Rw, —O—C(═O)-Rw, —O—C(═O)—O-Rw, and —S-Rw1, —C(═S)-Rw, —C(═S)—O-Rw, —C(═S)—S-Rw, —O—C(═S)—S-Rw, an alkynyl, an alkenyl where Rw is a C-1 to C-18 substituted or unsubstituted alkyl or a substituted or unsubstituted aryl and Rw1 is an unsubstituted or substituted aryl. Examples of advantageous substitution patterns are when Rew₂ is one of these electron withdrawing substituents and Rew, Rew₁, Rew₃, and Rew₄ are all hydrogen atoms. Specific examples of these substituted phenyl moieties would be 4-nitrophenyl, 4-cyanophenyl, 4-fluorophenyl, 4-chlorophenyl, 4-bromophenyl, 4-methylsulfonyl, 4-methylcarbonyl, 4-methoxycarbonyl, 4-(methylthio)carbonyl, 4-(methylsulfonyl)oxy, 4-acetoxy. Multiple substitution with these electron withdrawing group will further increase acidity, for instance Rew, Rew₁, Rew₂, Rew₃, and Rew₄ are all —F (a.k.a. pentafluorophenyl) or when two of the other electron withdrawing substituents especially those that are primarily electron withdrawing by resonance are present in the 2,4-position on the phenyl ring as non-limiting examples 2,4-dinitrophenyl, 2,4-dicyanophenyl, 2,4-di(methylsulfonyl)phenyl, 2, 4-di(methylcarbonyl)phenyl, 2,4-di(methoxycarbonylphenyl, 2-cyano-4-nitrophenyl, 2-nitro-4-cyanophenyl, 2-nitro-4-(methylsulfonyl)phenyl, 2-(methylsulfonyl)-4-nitrophenyl. For those electron withdrawing substituents with Rw alkyl moieties longer alkyl chains may be used to increase the solubility of these PAG and decrease the diffusion of the photoacid generated upon irradiation. Another approach to increase solubility of the PAG and limit diffusion of the photoacid released by having at least one of Rew, Rew₁, Rew₂, Rew₃, and Rew₄ as noted above as an alkyl substituent with a greater number of carbons, preferably C-4 to C-18. These alkyls may be chosen from linear alkyls, branched alkyls, or alicyclic alkyls with 1 or more rings. Non-limiting examples are when Rew, Rew₁, Rew₃, and Rew₄ are all hydrogen atoms and Rew₂ (which is in the 4-position on the phenyl ring (a.k.a. para) is a C-4 to C-8 unsubstituted alkyl substituent. Specific non-limiting examples would be 4-n-butylphenyl, 4-n-octylphenyl, 4-tertbutylphenyl, 4-cyclohexylphenyl, 4-(adamant-1-yl)-phenyl. More than one of Rew, Rew₁, Rew₂, Rew₃, and Rew₄ may be chosen to be a C-4 to C-8 alkyl to also increase solubility of the PAG and to limit its diffusion; as non-limiting examples 3,5-di(n-butyl)phenyl, 3,5-di(n-octyl)phenyl, 3,5-di(tert-amyl)phenyl and the like. Other substituents which can be used to limit diffusion. Another approach to increase solubility of the PAG and limit diffusion of the photoacid released by having at least one of Rew, Rew₁, Rew₂, Rew₃, and Rew₄, as noted above, be selected from a phenyl or a substituted phenyl substituent.

For substitution patterns on naphthaleneimido sulfate containing inventive PAGs where at least one of Rew, Rew₁, Rew₂, Rew₃, and Rew₄, is an electron withdrawing group such as NO₂, —OH, —O-Rw, —CN, —F, —Cl, —Br, —I, —S(═O)₂-Rw, —O—S (═O)₂-Rw, —O—S(═O)₂—O-Rw1, —O—S(═O)-Rw, —O—S(═O)—O-Rw1, —C(═O)—H, —C(═O)-Rw, —C(═O)—O-Rw, —C(═O)—S-Rw, —O—C(═O)—S-Rw, —O—C(═O)—O-Rw, —S-Rw1, —C(═S)-Rw, —C(═S)—O-Rw, —C(═S)—S-Rw, —O—C(═S)—S-Rw, —C(═S)-Rw, —C(═S)—O-Rw, —C(═S)—S-Rw, —O—C(═S)—S-Rw, an alkynyl, an alkenyl, or an additional naphthaleneimido sulfate moiety. Their UV sensitivity and solubility may be enhanced by employing the substitution patterns on the naphthalene moiety as shown in structures (Ig), (Ii), (Ii-1), (Iia), (Iia-1), (Iia′), (Iia′-1), (Iia-2), (Ij), (Ij-1), (Ija), (Ija-1), (Ij′a), (Ija′-1), (Ija-2), (Ija-3), (Ik), (Ik-1), (I1), (Il-1), (Im), (Im-1), (Ima), (Ima-1), (Imb), (Imc), (Imd), (In), (In-1), (Ina), (Inb), (Inc), or (Ind). FIG. 1a shows non-limiting examples of naphthaleneimido sulfate containing inventive PAGs an electron withdrawing substituent on the phenyl moiety.

FIG. 1b shows non-limiting examples of naphthaleneimido sulfate containing inventive PAGs which incorporate both a UV sensitivity moiety on the naphthalenic chromophore and an electron withdrawing substituent on the phenyl moiety.

As outlined herein, the inventive PAGs molecules may contain more than one N-(carbonylcarbamido) sulfate moiety. Particular embodiments of the bifunctional N-(carbonylcarbamido) aryl sulfate moiety as shown below in structures (II), (IIa), (IIb), (IIc), (IId) (IIe), (IIf), (IIo), (IIfa), (IIfa-1), (IIfa-2), (IIfa-3), (IIfa-4), (IIfa-5), (IIfa-7), (IIfa-8), (IIfa-9), (IIfb), (IIfb-1), (IIfb-2), (IIfb-3), (IIfb-4), (IIfb-5), (IIfb-6), (IIfb-7), (IIfb-8), (IIfb-9), (IIfc), (IIfc-1), (IIfc-2), (IIfc-3), (IIfc-4), (IIfc-5), (IIfc-6), (IIfc-7), (IIfc-8), and (IIfc-9), where the definition or R₁, R₂, R₁a, R′₁a, R₁b, R′₁b, R₁c, R′₁c, R₁d, R′₁d, Rar₁, Rar₂, Rar₃, Rar₄, Rar₅, Rar₆, Rar₇, Rar₈, Rar₉, Rar₁₀, Rnc₁, Rnc₂, X₁, Ralk Ro and Roi are defined as previously outlined. Since R₂ is an aryl moiety the presence of additional N-(carbonylcarbamido) sulfate moiety, an electron withdrawing substituent, will tend to increase the acidity of the released photoacid. For example, when the aryl moiety is a phenyl moiety as seen in Table 2 the acidity for phenyl hydrogen sulfate increases as a second N-(carbonylcarbamido) sulfate moiety is added either at the para or ortho position and increases further still with a third N-(carbonylcarbamido) sulfate moiety. Additionally, the presence of additional N-(carbonylcarbamido) sulfate moiety in these PAG may also tend to decrease diffusion because these will act either in their photolyzed form a photo released acid or in non-photolyzed form (if only one N (carbolycarbino) sulfate is photolyzed per PAG molecule). This increased acidity and reduced diffusion will be beneficial for resolution of negative or positive chemically amplified photoresists formulations in which the inventive PAGs are added. Structures (IIfa), (IIfb), and (IIfc) show specific examples of these when the aryl moiety is phenyl and the N-(carbonylcarbamido) sulfate chromophore is derived from 1,8-napththalimide. More specifically as non-limiting examples structures (IIfa-1) to (IIfa-9), (IIfb-1) to (IIb-9), and (IIc-1) to (IIc-9) show structures in which a pendant group has been added to tune UV absorbance and solubility.

TABLE 2

| Acid | pKₐ |
|---|---|
| 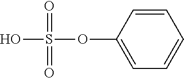 [SMILES: O=S(=O)(O)Oc1ccccc1] | −4.3 |
| 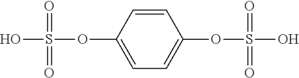 [SMILES: O=S(=O)(O)Oc1ccc(OS(=O)(=O)O)cc1] | −4.9 |
| 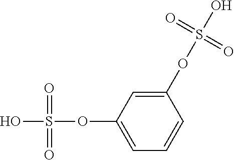 [SMILES: O=S(=O)(O)Oc1cccc(OS(=O)(=O)O)c1] | −4.8 |
| 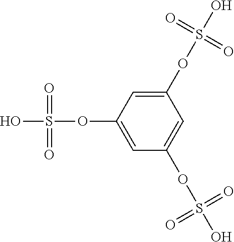 [SMILES: O=S(=O)(O)Oc1cc(OS(=O)(=O)O)cc(OS(=O)(=O)O)c1] | −5.7 |

(II) 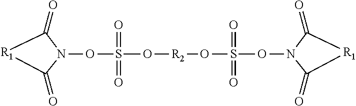
(IIa) 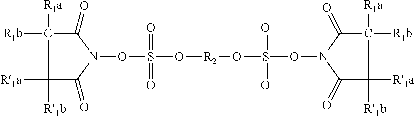
(IIb) 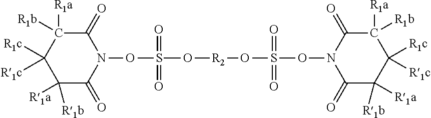
(IIc) 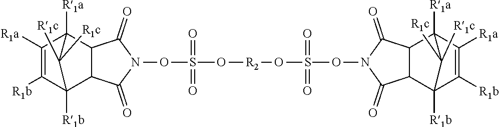
(IId) 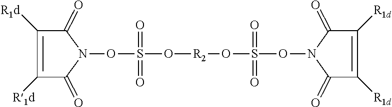
(IIe) 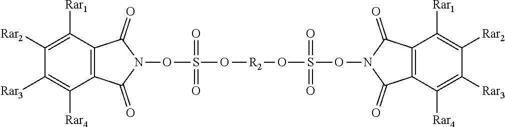
(IIf) 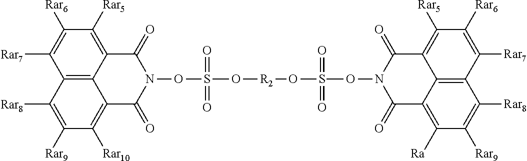
(IIfa) 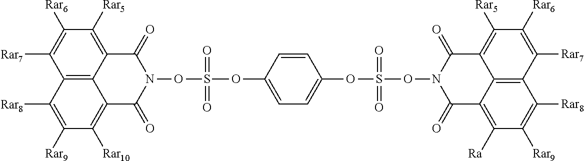
(IIfa-1) 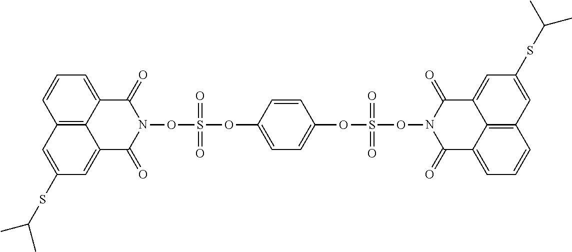 [SMILES: CC(C)Sc1cc2c3c(cccc3c1)C(=O)N(OS(=O)(=O)Oc1ccc(OS(=O)(=O)ON3C(=O)c4cccc5cc(SC(C)C)cc(c45)C3=O)cc1)C2=O]
(IIfa-2) 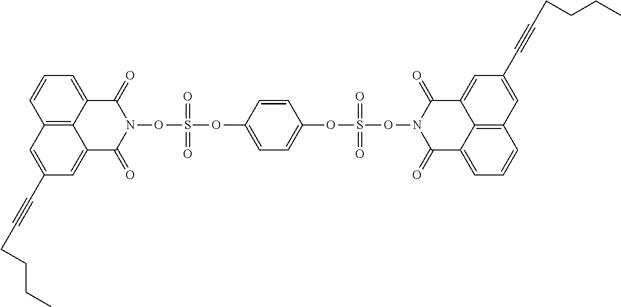 [SMILES: CCCCC#Cc1cc2c3c(cccc3c1)C(=O)N(OS(=O)(=O)Oc1ccc(OS(=O)(=O)ON3C(=O)c4cccc5cc(C#CCCCC)cc(c45)C3=O)cc1)C2=O]
(IIfa-3) 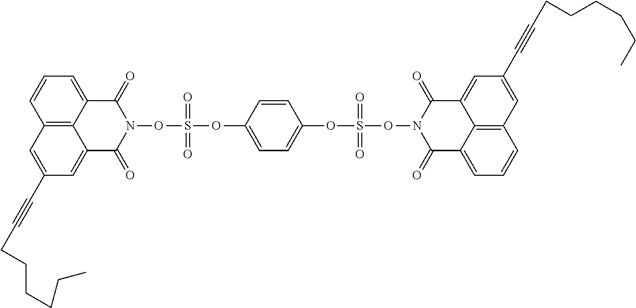 [SMILES: CCCCCCC#Cc1cc2c3c(cccc3c1)C(=O)N(OS(=O)(=O)Oc1ccc(OS(=O)(=O)ON3C(=O)c4cccc5cc(C#CCCCCCC)cc(c45)C3=O)cc1)C2=O]
(IIo) 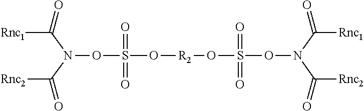
(IIfa-4) 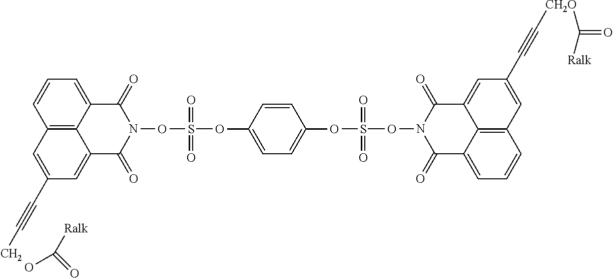
(IIfa-5) 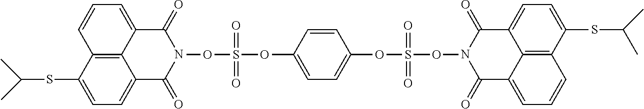 [SMILES: CC(C)Sc1ccc2c3c(cccc13)C(=O)N(OS(=O)(=O)Oc1ccc(OS(=O)(=O)ON3C(=O)c4cccc5c(SC(C)C)ccc(c45)C3=O)cc1)C2=O]
(IIfa-6) 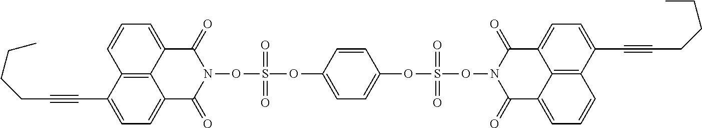 [SMILES: CCCCC#Cc1ccc2c3c(cccc13)C(=O)N(OS(=O)(=O)Oc1ccc(OS(=O)(=O)ON3C(=O)c4cccc5c(C#CCCCC)ccc(c45)C3=O)cc1)C2=O]
(IIfa-7) 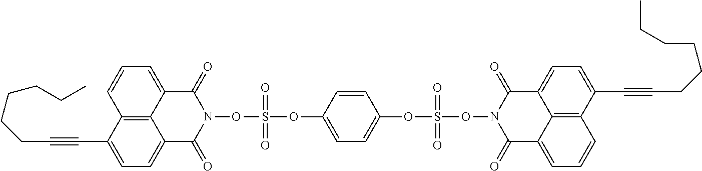 [SMILES: CCCCCCC#Cc1ccc2c3c(cccc13)C(=O)N(OS(=O)(=O)Oc1ccc(OS(=O)(=O)ON3C(=O)c4cccc5c(C#CCCCCCC)ccc(c45)C3=O)cc1)C2=O]
(IIfa-8) 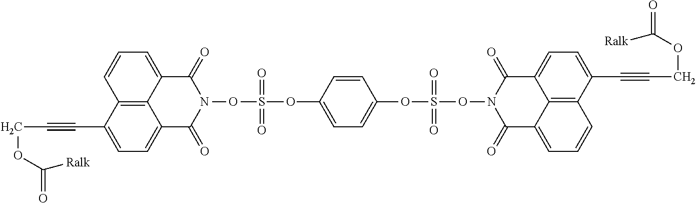
(IIfa-9) 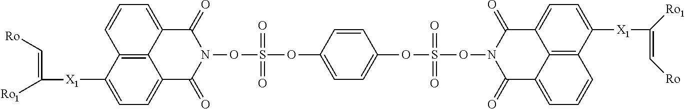
(IIfb) 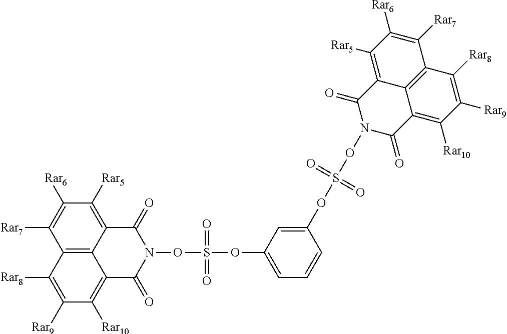
(IIfb-1) 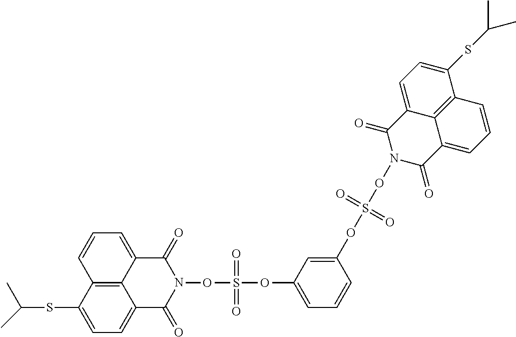 [SMILES: CC(C)Sc1ccc2c3c(cccc13)C(=O)N(OS(=O)(=O)Oc1cccc(OS(=O)(=O)ON3C(=O)c4cccc5c(SC(C)C)ccc(c45)C3=O)c1)C2=O]
(IIfb-2) 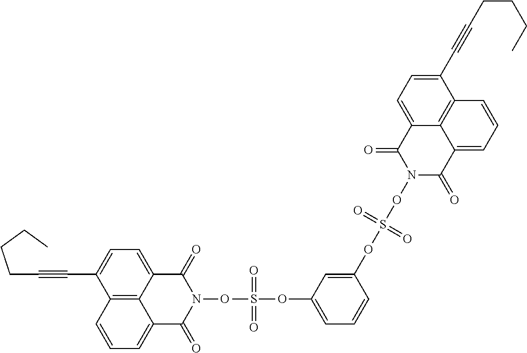 [SMILES: CCCCC#Cc1ccc2c3c(cccc13)C(=O)N(OS(=O)(=O)Oc1cccc(OS(=O)(=O)ON3C(=O)c4cccc5c(C#CCCCC)ccc(c45)C3=O)c1)C2=O]
(IIfb-3) 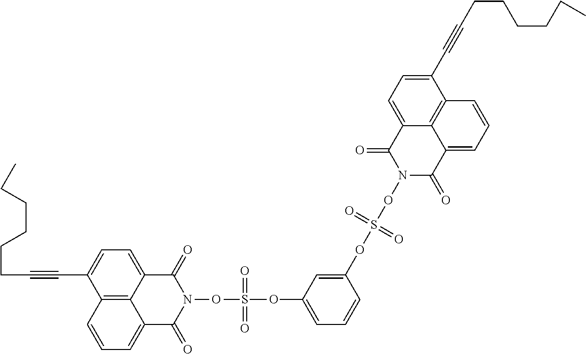 [SMILES: CCCCCCC#Cc1ccc2c3c(cccc13)C(=O)N(OS(=O)(=O)Oc1cccc(OS(=O)(=O)ON3C(=O)c4cccc5c(C#CCCCCCC)ccc(c45)C3=O)c1)C2=O]
(IIfb-4) 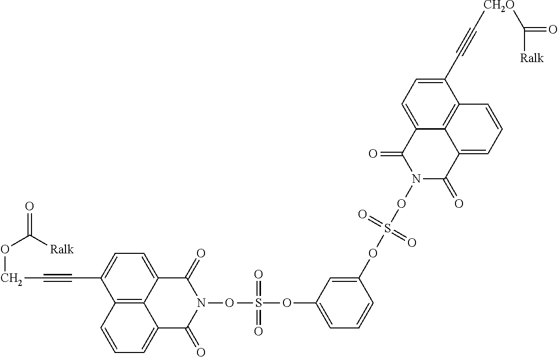
(IIfb-5) 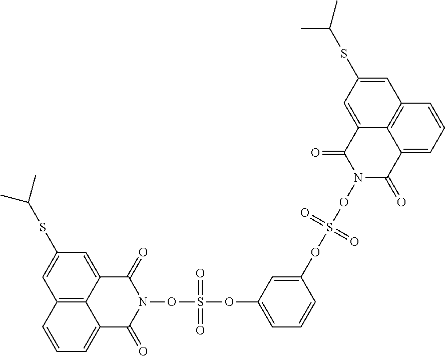 [SMILES: CC(C)Sc1cc2c3c(cccc3c1)C(=O)N(OS(=O)(=O)Oc1cccc(OS(=O)(=O)ON3C(=O)c4cccc5cc(SC(C)C)cc(c45)C3=O)c1)C2=O]
(IIfb-6) 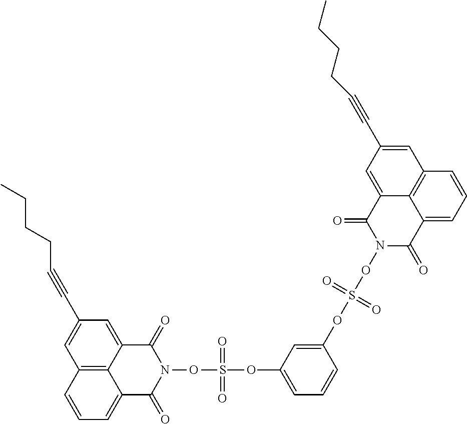 [SMILES: CCCCC#Cc1cc2c3c(cccc3c1)C(=O)N(OS(=O)(=O)Oc1cccc(OS(=O)(=O)ON3C(=O)c4cccc5cc(C#CCCCC)cc(c45)C3=O)c1)C2=O]
(IIfb-7) 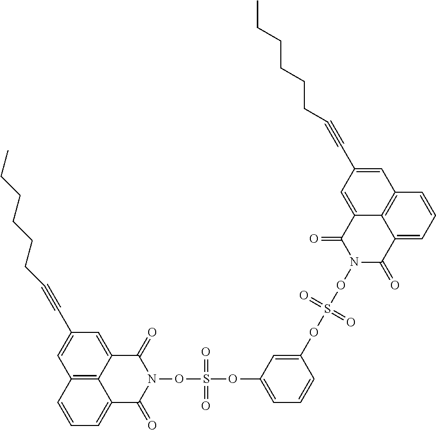 [SMILES: CCCCCCC#Cc1cc2c3c(cccc3c1)C(=O)N(OS(=O)(=O)Oc1cccc(OS(=O)(=O)ON3C(=O)c4cccc5cc(C#CCCCCCC)cc(c45)C3=O)c1)C2=O]
(IIfb-8) 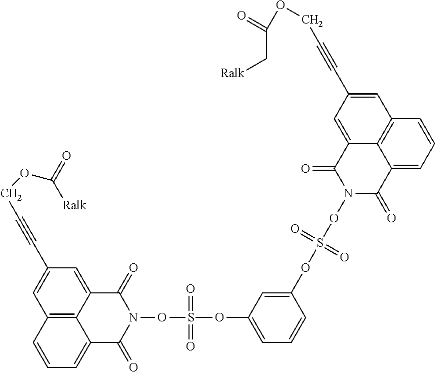
(IIfb-9) 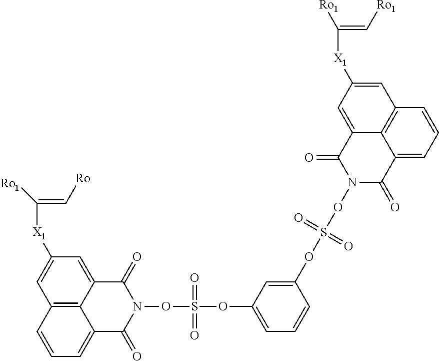
(IIfc) 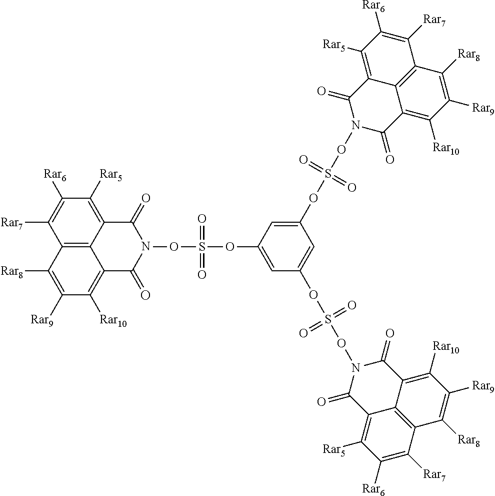
(IIfc-1) 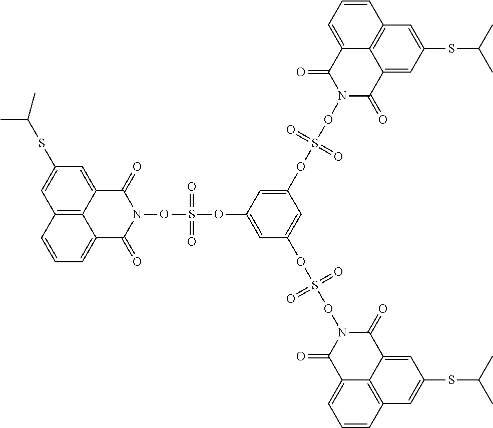 [SMILES: CC(C)Sc1cc2c3c(cccc3c1)C(=O)N(OS(=O)(=O)Oc1cc(OS(=O)(=O)ON3C(=O)c4cccc5cc(SC(C)C)cc(c45)C3=O)cc(OS(=O)(=O)ON3C(=O)c4cccc5cc(SC(C)C)cc(c45)C3=O)c1)C2=O]
(IIfc-2) 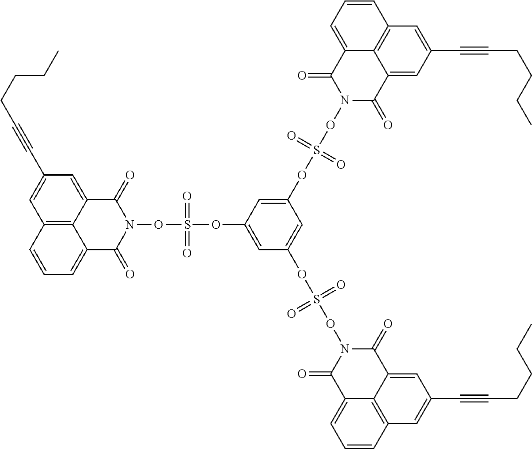 [SMILES: CCCCC#Cc1cc2c3c(cccc3c1)C(=O)N(OS(=O)(=O)Oc1cc(OS(=O)(=O)ON3C(=O)c4cccc5cc(C#CCCCC)cc(c45)C3=O)cc(OS(=O)(=O)ON3C(=O)c4cccc5cc(C#CCCCC)cc(c45)C3=O)c1)C2=O]
(IIfc-3) 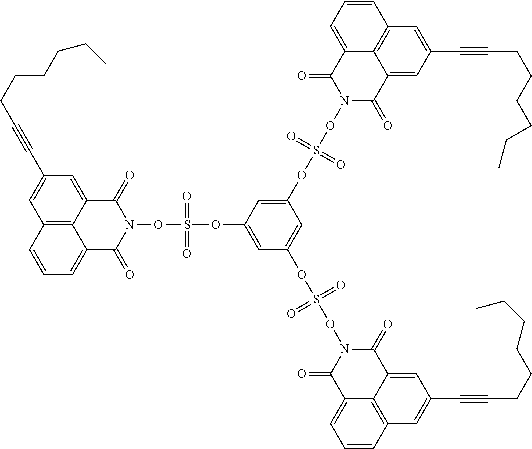 [SMILES: CCCCCCC#Cc1cc2c3c(cccc3c1)C(=O)N(OS(=O)(=O)Oc1cc(OS(=O)(=O)ON3C(=O)c4cccc5cc(C#CCCCCCC)cc(c45)C3=O)cc(OS(=O)(=O)ON3C(=O)c4cccc5cc(C#CCCCCCC)cc(c45)C3=O)c1)C2=O]
(IIfc-4) 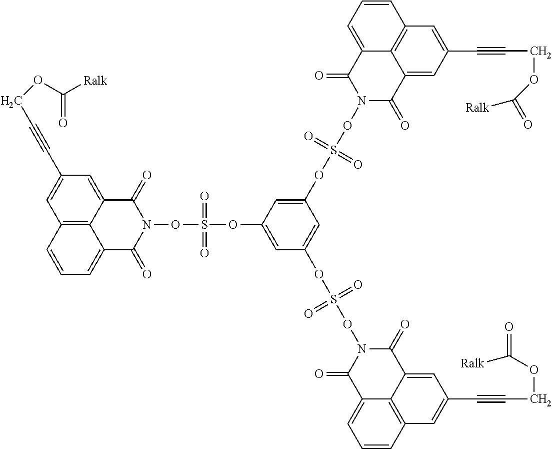
(IIfc-5) 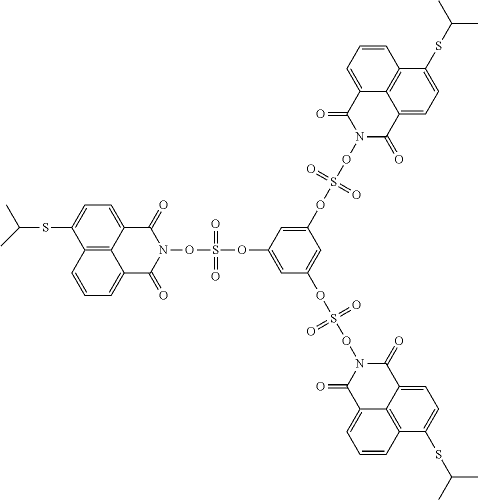 [SMILES: CC(C)Sc1ccc2c3c(cccc13)C(=O)N(OS(=O)(=O)Oc1cc(OS(=O)(=O)ON3C(=O)c4cccc5c(SC(C)C)ccc(c45)C3=O)cc(OS(=O)(=O)ON3C(=O)c4cccc5c(SC(C)C)ccc(c45)C3=O)c1)C2=O]
(IIfc-6) 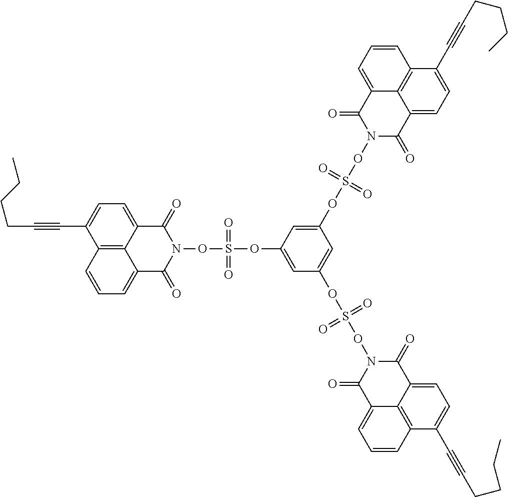 [SMILES: CCCCC#Cc1ccc2c3c(cccc13)C(=O)N(OS(=O)(=O)Oc1cc(OS(=O)(=O)ON3C(=O)c4cccc5c(C#CCCCC)ccc(c45)C3=O)cc(OS(=O)(=O)ON3C(=O)c4cccc5c(C#CCCCC)ccc(c45)C3=O)c1)C2=O]
(IIfc-7) 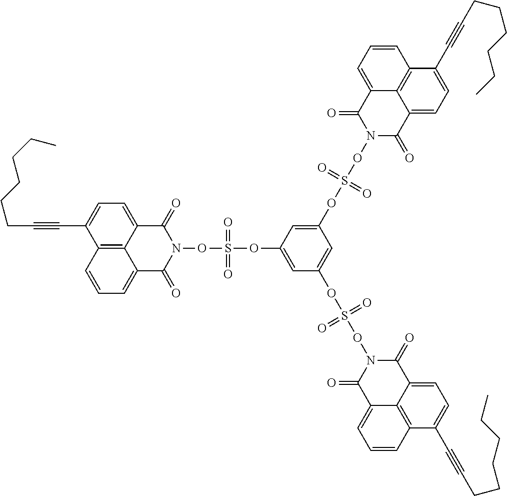 [SMILES: CCCCCCC#Cc1ccc2c3c(cccc13)C(=O)N(OS(=O)(=O)Oc1cc(OS(=O)(=O)ON3C(=O)c4cccc5c(C#CCCCCCC)ccc(c45)C3=O)cc(OS(=O)(=O)ON3C(=O)c4cccc5c(C#CCCCCCC)ccc(c45)C3=O)c1)C2=O]
(IIfc-8) 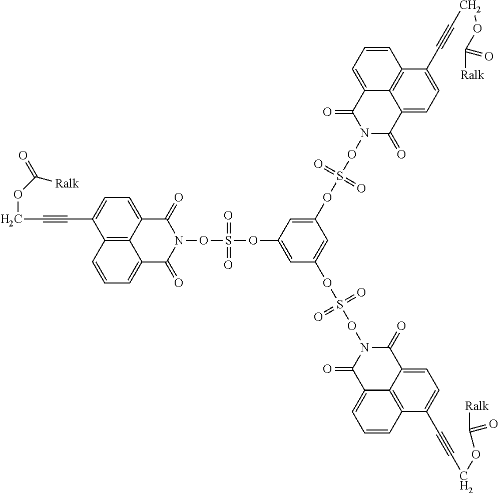
(IIfc-9)

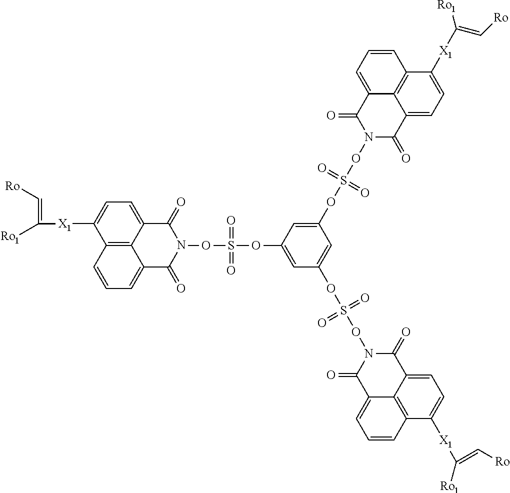

### Compositions

The inventive PAG described herein may be used in negative or positive chemically amplified photoresist which are designed to be sensitive to different radiation such as e-beam, 1-line UV, broadband UV, 248 nm UV, 193 nm UV (dry and immersion) and EUV. These formulations may contain one type of the novel PAGs described herein.

The loading of these PAGs will vary upon specific application and sensitivity required but in general the loading would be about 0.05 wt. % to about 10 wt. % of total wt. of solids (which includes all components except the organic spin casting solvent). In some instances, the loading of PAG may vary from about 0.05 wt. % to about 5 wt. % or from about 0.05 wt. % to about 2 wt. %, or about 0.05 to about 1 wt. %. In other instances, the loading of PAG may vary from about 0.1 wt. % to about 10 wt. % of total wt. of solids, or about 0.1 wt. % to about 5 wt. %, or about 0.1 wt. % to about 2 wt. %.

### Positive Chemically Amplified Photoresists

Another aspect of this invention is a positive chemically amplified photoresist composition developable in aqueous base, comprising,
1) a PAG component which is at least one inventive covalent PAG comprising a N-(carbonylcarbamido) aryl sulfate, as describe herein,
2) at least one polymer, comprising one or more repeat units with at least one acid cleavable group, which cleave upon the action of photogenerated acid from the covalent PAG comprising a N-(carbonylcarbamido) aryl sulfate and yield upon removal of the acid cleavable group base solubilizing groups which renders the polymer soluble in aqueous base developer such as 0.26 N aqueous tetramethyl ammonium hydroxide (TMAH). Examples of such base solubilizing groups are phenols, carboxylic acid, phosphonic acids, and 1,1,1-trifluoro-2-(perfluoalkyl)-alkanyl-2-ols such as 1,1,1-trifluoro-2-(trifluoromethyl)-methanyl-2-ol. In one aspect of this component, it is at least one polymer comprising one or more (meth)acrylate repeat units and further comprising one or more repeat units with at least one acid cleavable group which when cleaved by photogenerated acid renders said polymer soluble in aqueous base developer. 11) an organic spin coating solvent.

These positive chemically amplified photoresists may additionally comprise any one the following optional components or mixture of these components:
An optional resin component which is soluble in 0.26 N aqueous TMAH,
An optional DNQ PAC component,
An optional thiol derivative component where the thiol moiety is attached to an sp2 carbon which is part of the ring, wherein said thiol derivative is selected from the group consisting of heterocyclic thiol compound and an aryl thiol compound,
An optional glycidyl hydroxy benzoic acid condensate additive,
An optional base component,
An optional photobleaching dye component,
An optional sensitizer,
An optional different type of PAG component,
An optional surfactant component.

Both the essential components and optional components for positive chemically amplified photoresists will be described in more detail as follows.

### Negative Chemically Amplified Photoresists

Another aspect of this invention is a negative chemically amplified photoresist composition comprising:
1a) a PAG component which is at least one inventive covalent PAG comprising a N-(carbonylcarbamido) aryl sulfate, as describe herein,
2a) a photoresist resin component which is soluble in aqueous base which undergoes chemically amplified crosslinking in the presence of a photogenerated acid from the inventive covalent PAG comprising a N-(carbonylcarbamido) aryl sulfate, as described herein. This photoresist resin may be a single base soluble resin which can self-crosslink cationically under the influence of a said photogenerated acid or alternatively a more complex component which comprises at least one resin which is soluble in 0.26 N aqueous TMAH and at least one latent electrophilic crosslinker which upon action with the photoacid generator can cationically crosslink said resin. 13a) an organic spin coating solvent.

These negative chemically amplified photoresists may additionally comprise any one of the following optional components or mixtures of these:
An optional resin component which is soluble in 0.26 N aqueous TMAH,
An optional crosslinking component,
An optional DNQ PAC component,
An optional thiol derivative component where the thiol moiety is attached to an sp2 carbon which is part of the ring, wherein said thiol derivative is selected from the group consisting of heterocyclic thiol compound and an aryl thiol compound,
An optional base component,
An optional photobleaching dye component,
An optional dye,
An optional different type of PAG component
An optional sensitizer component,
An optional surfactant component.

Both the essential components and optional components for positive and negative chemically amplified photoresists will be described in more detail as follows.

### Detailed Description of Components for Positive and Negative Chemically Amplified Photoresists

### Polymers Comprising One or More Repeat Units with at Least One Acid Cleavable Group

The polymer of the photoresist composition of the present invention which are useful for positive chemically amplified photoresist are ones which are insoluble in an aqueous alkali developer but become soluble prior to development. Typically, the polymer is an aqueous alkali soluble polymer which is protected by an acid labile group. Alkali soluble polymers can be homopolymers or copolymers comprising units derived from monomers comprising a hydroxy group or an ester group. Preferred, are polymers comprising phenolic groups, such as polymer repeat unit derived from the hydroxystyrene monomer or aqueous base soluble Novolak polymers. The phenolic groups are blocked with an acid labile group, such as esters and/or acetals, tert-butoxycarbonyl or alkyloxycarbonylalkyl (such as (tert-butoxycarbonyl)methyl). Also preferred are (alkyl)acrylates which may be copolymerized to provide an acid labile ester group, examples of which are tert-butyl acrylate, tert-butyl methacrylate and methyladamantyl acrylate. An example are copolymers of hydroxystyrene and acrylates. The polymers may further comprise comonomeric units which do not have acid labile groups and are derived from polymerizable monomers, for example, styrene, acetoxystyrene, benzyl methacrylate, methoxystyrene and acrylate monomers which have pendant alcoholic group such as 2-hydroxyethylmethacrylate (HEMA) and the like.

Examples of hydroxystyrene based resins usable for capping with acid labile groups include poly-(4-hydroxystyrene); poly-(3-hydroxystyrene); poly-(2-hydroxystyrene); and copolymers of 4-, 3-, or 2-hydroxystyrene with other monomers, particularly dipolymers and terpolymers. Examples of other monomers usable herein include 4-, 3-, or 2-acetoxystyrene, 4-, 3-, or 2-alkoxystyrene, styrene, α-methylstyrene, 4-, 3-, or 2-alkylstyrene, 3-alkyl-4-hydroxystyrene, 3,5-dialkyl-4-hydroxystyrene, 4-, 3-, or 2-chlorostyrene, 3-chloro-4-hydroxystyrene, 3,5-dichloro-4-hydroxystyrene, 3-bromo-4-hydroxystyrene, 3,5-dibromo-4-hydroxystyrene, vinylbenzyl chloride, 2-vinylnaphthalene, vinylanthracene, vinylaniline, vinylbenzoic acid, vinylbenzoic acid esters, N-vinylpyrrolidone, 1-vinylimidazole, 4-, or 2-vinylpyridine, 1-vinyl-2-pyrrolidinone, N-vinyl lactam, 9-vinylcarbazole, vinyl benzoate, acrylic acid and its derivatives, i.e. methyl acrylate and its derivatives, acrylamide and its derivatives, methacrylic acid and its derivatives, i.e. methyl methacrylate and its derivatives, methacrylamide and its derivatives, acrylonitrile, methacrylonitrile, 4-vinyl benzoic acid and its derivatives, i.e. 4-vinyl benzoic acid esters, 4-vinylphenoxy acetic acid and its derivatives, i.e. 4-vinylphenoxy acetic acid esters, maleimide and its derivatives, N-hydroxymaleimide and its derivatives, maleic anhydride, maleic/fumaric acid and their derivatives, i.e. maleic/fumaric acid ester, vinyltrimethylsilane, vinyltrimethoxysilane, or vinyl-norbornene and its derivatives. Another examples of preferred other monomers usable herein include isopropenylphenol, propenylphenol, poly-(4-hydroxyphenyl) (meth)acrylate, poly-(3-hydroxyphenyl) (meth)acrylate, poly-(2-hydroxyphenyl) (meth)acrylate, N-(4-hydroxyphenyl) (meth)acrylamide, N-(3-hydroxyphenyl) (meth)acrylamide, N-(2-hydroxyphenyl) (meth)acrylamide, N-(4-hydroxybenzyl) (meth)acrylamide, N-(3-hydroxybenzyl) (meth)acrylamide, N-(2-hydroxybenzyl) (meth)acrylamide, 3-(2-hydroxy-hexafluoropropyl-2)-styrene, and 4-(2-hydroxy-hexafluoropropyl-2)-styrene.

As described above, for the photoresist of the present invention, the hydroxystyrene based resin is made alkali insoluble by protecting alkali soluble groups on the resin with an acid cleavable protective group. The introduction of the protective group may be carried out by any proper method depending upon alkali soluble groups on the resin, and could be easily carried out by a person having ordinary skill in the art. Similarly, an aqueous base soluble Novolak resin may be made insoluble in aqueous base by protecting some or all the phenolic moieties with an acid labile group, which upon cleavage release the aqueous base soluble Novolak.

For example, when the alkali soluble group on the resin is a phenolic hydroxy group such as in 4-hydoxystyrene copolymers or Novolaks, the phenolic hydroxy groups present in the resin are partly or fully protected by any known acid labile protective group, preferably by one or more protective groups which form acid cleavable C(O)OC, C—O—C or C—O—Si bonds. Examples of protective groups usable herein include acetal or ketal groups formed from alkyl or cycloalkyl vinyl ethers, silyl ethers formed from suitable trimethylsilyl or t-butyl(dimethyl)silyl precursors, alkyl ethers formed from methoxymethyl, methoxyethoxymethyl, cyclopropylmethyl, cyclohexyl, t-butyl, amyl, 4-methoxybenzyl, o-nitrobenzyl, or 9-anthrylmethyl precursors, t-butyl carbonates formed from t-butoxycarbonyl precursors, and carboxylates formed from t-butyl acetate precursors. Also useful are groups such as (tert-butoxycarbonyl)methyl and its (C-1 to C-6) alkyl analogs.

When the alkali soluble group on the resin is a carboxyl group, the carboxyl groups present on the resin are partly or fully protected by an acid labile protective group, preferably by one or more protective groups which form acid cleavable C—O—C or C—O—Si bonds. Examples of protective groups usable herein include alkyl or cycloalkyl vinyl ethers and esters formed from precursors containing methyl, methyloxymethyl, methoxyethoxymethyl, benzyloxymethyl, phenacyl, N-phthalimidomethyl, methylthiomethyl, t-butyl, amyl, cyclopentyl, 1-methylcyclopentyl, cyclohexyl, 1-methylcyclohexyl, 2-oxocyclohexyl, mevalonyl, diphenylmethyl, α-methylbenzyl, o-nitrobenzyl, p-methoxybenzyl, 2,6-dimethoxybenzyl, piperonyl, anthrylmethyl, triphenylmethyl, 2-methyladamantyl, tetrahydropyranyl, tetrahydrofuranyl, 2-alkyl-1,3-oxazolinyl, trimethylsilyl, or t-butyldimethylsilyl group.

According to the present invention, the above resins may be used alone or a mixture of two or more.

Particularly preferred for 248 nm and 193 nm applications are polymers comprising units derived from at least one monomer selected from substituted hydroxystyrene, unsubstituted hydroxystyrene, substituted alkyl acrylates, unsubstituted acrylates. The acrylates may contain acid labile groups or nonacid labile groups. The polymer may further comprise units which do not have an acid labile group, such as those derived from monomers based on substituted or unsubstituted styrene, ethylene with pendant groups such as C-5 to C-10 monocyclic alicyclic alkyls, multicyclic alicyclic alkyls, such as adamantly, norbornanyl, octahydro-1H-methanoinden-5-yl, aryls such as phenyl, carboxylic acid, etc. For i-line and broadband application two types of polymers are preferred, Novolak types and acrylate types which contain acid cleavable groups protecting at least partially the base solubilizing OH moieties of these polymer. In the Novolak and Acrylate type polymers respectively their phenolic and carboxylic acid base solubilizing hydroxy moieties are partly or fully protected by an acid labile protective group, preferably by one or more protective groups which form acid cleavable C—O—C or C—O—Si bonds. Examples of protective groups usable herein include alkyl or cycloalkyl vinyl ethers and esters formed from precursors containing methyl, methyloxymethyl, methoxyethoxymethyl, benzyloxymethyl, phenacyl, N-phthalimidomethyl, methylthiomethyl, t-butyl, amyl, cyclopentyl, 1-methylcyclopentyl, cyclohexyl, 1-methylcyclohexyl, 2-oxocyclohexyl, mevalonyl, diphenylmethyl, α-methylbenzyl, o-nitrobenzyl, p-methoxybenzyl, 2,6-dimethoxybenzyl, piperonyl, anthrylmethyl, triphenylmethyl, 2-methyladamantyl, tetrahydropyranyl, tetrahydrofuranyl, 2-alkyl-1,3-oxazolinyl, trimethylsilyl, or t-butyldimethylsilyl group. Although the plasma etch resistance needed for photoresist pattern transfer is inherent in these Novolak type polymers for the acrylate type polymers this etch resistance may is imparted by the inclusion of repeat units which contain aryl moieties. Example of such repeat units are styrene and substituted styrenes, acrylate or methacrylate repeat units with pendant benzyl moieties or substituted pendant benzyl moieties on acrylate or methacrylates with pendant oxyphenyl moieties or substituted oxy phenyl moieties. Another approach are methacrylate or acrylate polymer which do not contain any acid labile groups on the acrylate functionality but instead include a hydroxystyrene derived repeat unit whose base solubilizing moiety is protected with one of the aforementioned protecting group. Hybrid materials are also possible that contain both acrylate and/or methacrylate derived from the corresponding acid, whose carboxylic acid moieties are partially or fully protected with an acid labile group but also contain repeat units derived from hydroxystyrene whose phenolic base solubilizing groups is are partially or protected with an acid labile group. Mixtures of these different types of polymers may also be employed.

### Photoresist Resin Component which is Soluble in Aqueous Base which Undergoes Chemically Amplified Crosslinking

This component for use in negative chemically amplified photoresists is an aqueous base soluble phenolic resin which also contains pendant groups which have a latent electrophile which can be activated through the action of a photoacid and promote the crosslinking of the resin with itself. An example of such materials are copolymers of 4-hydroxystyrene with a 4-vinylbenzyl carboxylate such as 4-vinylbenzyl acetate. Alternatively, such systems may additionally comprise a latent cationic crosslinker. Another embodiment are aqueous base soluble resins where this component may comprise two components, one component of which is a base soluble phenolic polymer such as Novolaks or copolymers comprising 4-hydroxystyrene which do not have a latent electrophilic site but where the crosslinking reaction with a separate latent electrophilic crosslinker, as described herein. FIG. 2 shows some examples of polymers and crosslinkers which contain latent electrophiles.

### Optional Resin Component, which is Soluble in 0.26 N Aqueous TMAH

The optional resin component which is soluble in 0.26N aqueous TMAH, which may be used in the inventive chemically amplified photoresist compositions may be at least one copolymer comprising base solubilizing repeat units derived from methacrylic or acrylic acid, copolymers which comprise base solubilizing repeat units derived from hydroxystyrene, alone or copolymerized with other repeat units, copolymer containing mixtures of repeat units selected from the group consisting of ones derived from 4-hydroxystyrene, methacrylic acid, acrylic acid. The optional resin which is soluble in 0.26 N aqueous TMAH may also be a mixture of two of these types of resins or mixtures with other types of base soluble resins such as Novolak resin. Novolak polymers may be used as the optional resin components soluble in aqueous bases either are one such polymer or mixtures of two or more of these polymers or mixtures of Novolak polymers with other types of base soluble resins. Non-limiting examples are base soluble Novolaks comprising repeat units having bridges and phenolic compounds. Suitable phenolic compounds include, without limitation, phenols, cresols, substituted and unsubstituted resorcinols, xylenols, substituted and unsubstituted benzene triols, and combinations thereof. Specific non-limiting examples of suitable phenols are Bisphenol A, Bisphenol F, Bisphenol AP, Bisphenol AF, Bisphenol B, Bisphenol BP, Bisphenol C, Bisphenol E, Bisphenol S, phenol, meta-cresol, para-cresol, ortho-cresol, 3,5-dimethylphenol, 3-ethylphenol, 4-ethylphenyl, 3,5-diethylphenol, and combinations thereof. Novolak polymers are produced, usually, with an acid catalyst, by condensation polymerization of phenolic compounds and aldehydes such as formaldehyde, acetaldehyde or substituted or unsubstituted benzaldehydes or condensation products of phenolic compounds and substituted or unsubstituted methylol compounds. Bridges described supra may comprise methylene groups or methyne groups. Novolak polymers can also be made as condensation products of ketones such as acetone, methyl ethyl ketone, acetophenone and the like. Catalysts may include Lewis acids, Bronsted acids, dicationic and tricationic metal ions and the like. For example, without limitation, aluminum chloride, calcium chloride, manganese chloride, oxalic acid, hydrochloric acid, sulfuric acid, methane sulfonic acid trifluoromethane sulfonic acid or combinations comprising any of the foregoing may be used. Examples of suitable Novolak polymers include those obtained by the condensation reaction between a phenolic compound such as phenol, o-cresol, m-cresol, p-cresol, 2-5-xylenol, Bisphenol A, bisphenol F, Bisphenol AP, Bisphenol AF, Bisphenol B, Bisphenol BP, Bisphenol C, Bisphenol E, Bisphenol S, phenol, 3,5-dimethylphenol, 3-ethylphenol, 4-ethylphenyl, 3,5-diethylphenol, and the like with an aldehyde compound such as formaldehyde in the presence of an acid or multivalent metal-ion catalyst. An exemplary weight average molecular weight for the alkali-soluble Novolak polymer may be in the range from 1,000 to 30,000 Daltons. A further exemplary weight average molecular weight may be from 1,000 to 20,000 Daltons. A still further exemplary weight average molecular weight may be from 1,500 to 10,000 Daltons. Exemplary bulk dissolution rates for Novolak polymers in 2.38% aqueous tetramethylammonium hydroxide are 10 Å/sec (Angstrom units per second) to 15,000/sec. Further exemplary bulk dissolution rates are 100 Å/sec to 10,000 Å/sec. Still further exemplary bulk dissolution rates are 200/sec to 5,000/sec. A still further exemplary bulk dissolution rate of 1,000/sec may be obtained from a single Novolak polymer or a blend of Novolak polymers, each comprising m-cresol repeat units. Exemplary cresylic Novolak polymers may comprise, in cresol mole percentage terms, 0%-60% p-cresol, 0%-20% o-cresol, and 0%-80% m-cresol. Further exemplary cresylic Novolak polymers may comprise 0%-50% p-cresol, 0%-20% o-cresol, and 50%-100% m-cresol. Repeat units in Novolak polymers are defined by the composition of the polymer, so that, for example, p-cresol may be introduced by polymerization with an aldehyde or by dimethylol-p-cresol. Moreover, cresylic Novolak polymers may contain other phenolic compounds such as phenol, xylenols, resorcinols, benzene triols and the like.

In one embodiment this base soluble Novolak is derived from the copolymerization of Bisphenol A, formaldehyde, and meta-cresol. The binder resin comprises repeat units having general structure (NOV-1), wherein q represents the number of repeat units in the polymer chain and attachment of the —CH₂— repeat unit may be at ortho or para positions. This component may also contain branched structures in which an additional 1 to 2 ortho-positions on the repeat unit derived from Bisphenol A are linked to a —CH₂— repeat unit attached to another repeat unit derive from either meta-cresol or Bisphenol-A. Further, an additional meta or para position on the repeat unit derived from meta-cresol can be linked to a —CH₂— repeat unit attached to another repeat unit derived from either meta-cresol or Bisphenol A.

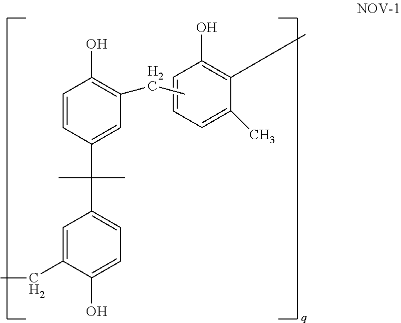

In one embodiment of the Novolak, having structure (NOV-1), it has an Mw ranging from about 20,000 to about 5,000 with a polydispersity (PD) ranging from about 3 to about 5. In another embodiment of this aspect of the invention the Novolak has an Mw ranging from about 15,000 to about 3,000. In another embodiment Mw is from about 12,000 to about 5,000. In yet another embodiment Mw is from about 11,000 to about 7,000. In still another embodiment Mw is from about 10,000 to about 8,000. In another embodiment, the Mw is about 9,000 and the PD is about 4.1.

In positive chemically amplified photoresist compositions this optional component may be used as supplemental component to tune the dissolution and plasma etching characteristics of such photoresists. In negative chemically amplified photoresist this component may play a similar dissolution role but additionally when used in conjunction with latent cationic crosslinker induce by the action of the photoacid a crosslinking reaction.

### Optional Diazonaphthoquinonesulfonate Photoactive Compound (DNQ PAC)

For the positive and negative chemically amplified photoresist composition described herein which contain the inventive covalent PAG comprising a N-(carbonylcarbamido)oxy aryl sulfonate as described herein, these composition may further comprise a DNQ-PAC component which may be a single material or a mixture of materials in which is Diazonaphthoquinonesulfonate moiety having structure (1-DNQ) or a structure (2-DNQ) which forms at least one sulfonate ester with a phenolic compound.

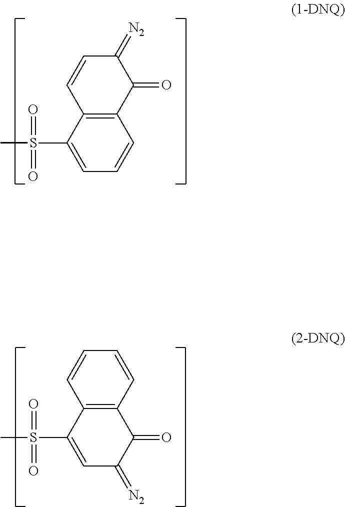

In another embodiment of the inventive compositions described herein, said DNQ PAC ranges from about 5 to about 20 wt. % solids.

In another embodiment of the inventive compositions described herein, said DNQ PAC component is a single material or a mixture of materials having general formula (3-DNQ) wherein D_{1c}, D_{2c}, D_{3c} and D_{4c} are independently selected from H or a moiety having structure (1-DNQ), and further wherein at least one of D_{1c}, D_{2c}, D_{3c} or D_{4c} is a moiety having structure (1-DNQ).

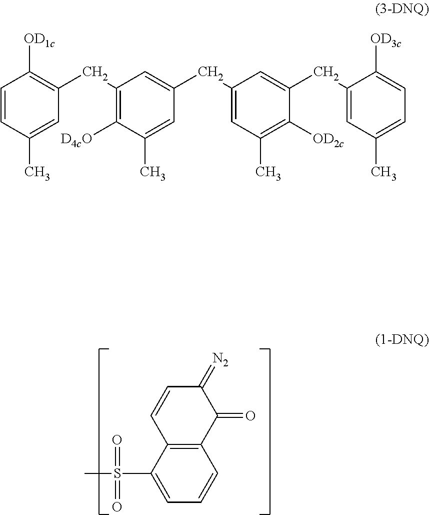

In another embodiment of the inventive compositions described herein, said DNQ PAC component is a single material or a mixture of materials having general formula (3-DNQ) wherein D_{1c}, D_{2c}, D_{3c} and D_{4c} are independently selected from H or a moiety having structure (2-DNQ), and further wherein at least one of D_{1c}, D_{2c}, D_{3c} or D_{4c} is a moiety having structure (2-DNQ).

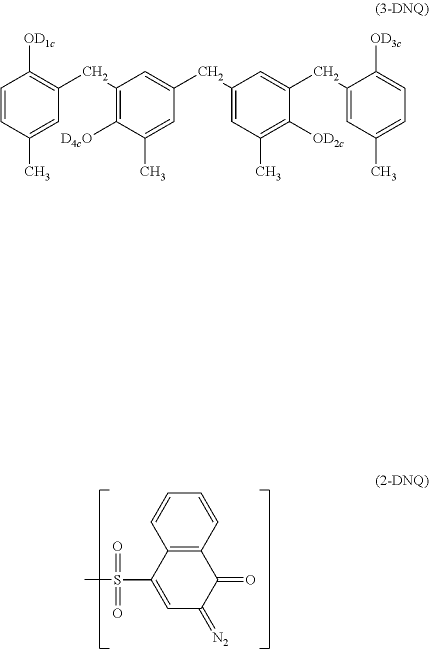

In another embodiment of the inventive compositions described herein, said DNQ PAC component is a single material or a mixture of materials having general formula (4-DNQ), wherein D_{1c}, D_{2c}, and D_{3c} are independently selected from H or a moiety having structure (1-DNQ), and further wherein at least one of D_{1c}, D_{2c}, or D_{3c} is a moiety having structure (1-DNQ),

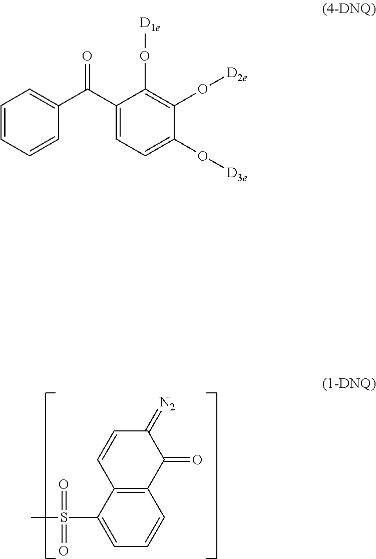

In another embodiment of the inventive compositions described herein, said DNQ PAC component is a single material or a mixture of materials having general formula (4-DNQ), wherein D_{1c}, D_{2c}, and D_{3c} are independently selected from H or a moiety having structure (2-DNQ), and further wherein at least one of D_{1c}, D_{2c}, or D_{3c} is a moiety having structure (2-NDQ).

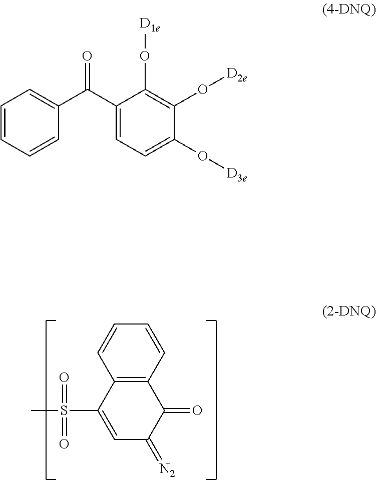

In another embodiment of the inventive compositions described herein, said DNQ PAC component is a single material or a mixture of materials having general formula (4a-DNQ), wherein D_{1c}, D_{2c}, D_{3c} and D₄ₑ are independently selected from H or a moiety having structure (1-DNQ), and further wherein at least one of D_{1c}, D_{2c}, D_{3c} and D₄ₑ is a moiety having structure (1-DNQ).

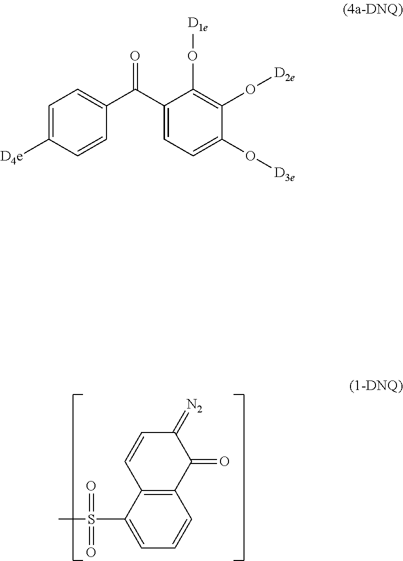

In another embodiment of the inventive compositions described herein, said DNQ PAC component is a single material or a mixture of materials having general formula (4a-DNQ), (wherein D_{1c}, D_{2c}, D_{3c} and D₄ₑ are independently selected from H or a moiety having structure(2-DNQ), and further wherein at least one of D_{1c}, D_{2c}, D_{3c} and D₄ₑ is a moiety having structure (2-DNQ).

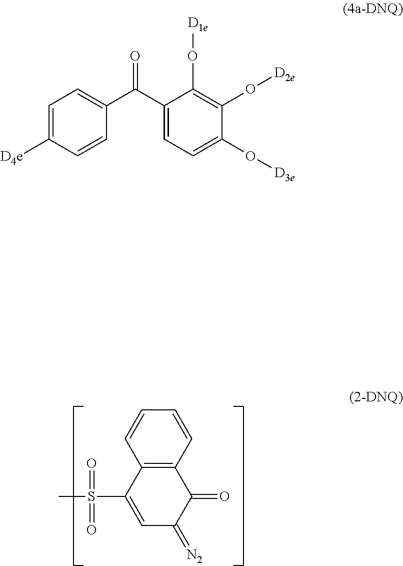

In another embodiment of the inventive compositions described herein, said DNQ PAC component is a single material or a mixture of materials having general formula (5-DNQ), wherein D_{1f}, D_{2f}, D_{3f} and D_{4f} are independently selected from H or a moiety having structure (1-DNQ), and further wherein at least one of D_{1f}, D_{2f}, D_{3f} or D_{4f} is a moiety having structure (1-DNQ).

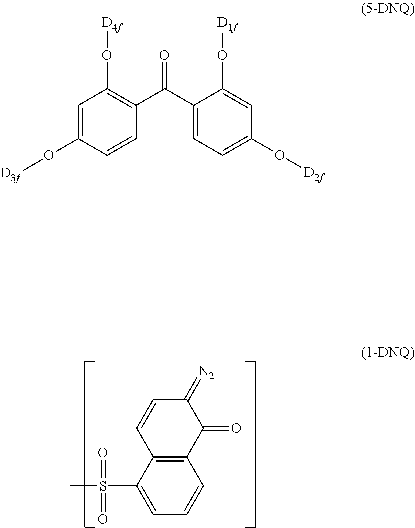

In another embodiment of the inventive compositions described herein, said DNQ PAC component is a single material or a mixture of materials having general formula (5-DNQ), wherein D_{1f}, D_{2f}, D_{3f} and D_{4f} are independently selected from H or a moiety having structure (2-DNQ), and further wherein at least one of D_{1f}, D_{2f}, D_{3f} or D_{4f} is a moiety having structure (2-DNQ).

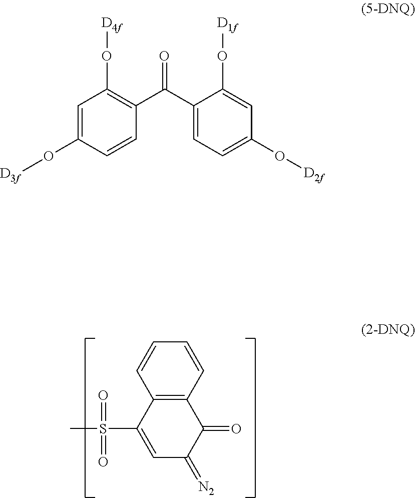

In another embodiment of the inventive compositions described herein, said DNQ PAC component is a mixture of the above described DNQ PAC materials.

The following are non-limiting examples of suitable DNQ PAC for use in the disclosed inventive composition as component c) said DNQ PAC component: PW898 (CAS 107761-81-9) is a 2,2′-4,4-tetrahydroxy-DNQ PAC (6-diazo-5,6-dihydro-5-oxo-1-naphthalene-sulfonic acid ester with (4-hydroxyphenyl)-(2,3,4-trihydroxyphenyl), methanone) available from Accel Pharmtech LLC (East Brunswick, NJ). It is a mixture of materials having general formula (4a-DNQ), wherein D_{1c}, D_{2c}, D_{3c} and D₄ₑ are independently selected from H or a moiety having structure (1-DNQ), and further wherein at least one of D_{1c}, D_{2c}, D_{3c} or D₄ₑ is a moiety having structure (1-DNQ).

NK-280 is a DNQ-PC sold under this name by TOYO GOSEI., LTD.

It is a mixture of materials having general formula (3-DNQ) wherein D_{1c}, D₂c, D_{3c} and D_{4c} are independently selected from H or a moiety having structure (1-DNQ), where at least one of D_{1c}, D_{2c}, D_{3c}, or D_{4c} is a moiety having structure (1-DNQ) and on average about 2.8 of the phenolic positions D_{1c}, D_{2c}, D_{3c} and D_{4c} groups are esterified with (1-DNQ). In the aspect of said inventive positive chemically amplified photoresist FIG. 3 shows non-limiting examples of DNQ PAC compounds which may be used to form a free PAC component and/or be used to form a PAC moiety attached to the polymer component on a phenolic moiety through an acetal comprising linking group.

### Optional Thiol Derivative Component

The positive and negative chemically amplified photoresist composition described herein which contain the inventive covalent PAG comprising a N-(carbonylcarbamido) aryl sulfate as described herein, may additionally contain a thiol derivative component this component which may be selected from the group consisting of thiol derivatives having the structures (H1), (H2) (H3), or (H4), wherein in said structure (H1), Xt is selected from the group consisting of N(Rt₃), C(Rt₁)(Rt₂), O, S, Se, and Te;
in said structure (112), Y is selected from the group consisting of C(Rt₃) and N;
in said structure (H3), Z is selected from the group consisting of C(Rt₃) and N; and in said structure (H4), where Arene is selected from phenyl, a substituted phenyl, an unsubstituted polycyclic arene moiety and a substituted polycyclic arene moiety, Rt₁, Rt₂, and Rt₃ are independently selected from the group consisting of H, a substituted alkyl group having 1 to 8 carbon atoms, an unsubstituted alkyl group having 1 to 8 carbon atoms, a substituted alkenyl group having 2 to 8 carbon atoms, an unsubstituted alkenyl group having 2 to 8 carbon atoms, a substituted alkynyl group having 2 to 8 carbon atoms, an unsubstituted alkynyl group having 2 to 8 carbon atoms, a substituted aromatic group having 6 to 20 carbon atoms, a substituted heteroaromatic group having 3 to 20 carbon atoms, an unsubstituted aromatic group having 6 to 20 carbon atoms and an unsubstituted heteroaromatic group having 3 to 20 carbon atoms,
Rt₄ is independently selected from the group consisting of H, OH, a substituted alkyl group having 1 to 8 carbon atoms, an unsubstituted alkyl group having 1 to 8 carbon atoms, a substituted alkenyl group having 2 to 8 carbon atoms, an unsubstituted alkenyl group having 2 to 8 carbon atoms, a substituted alkynyl group having 2 to 8 carbon atoms, an unsubstituted alkynyl group having 2 to 8 carbon atoms, a substituted aromatic group having 6 to 20 carbon atoms, a substituted heteroaromatic group having 3 to 20 carbon atoms, an unsubstituted aromatic group having 6 to 20 carbon atoms and an unsubstituted heteroaromatic group having 3 to 20 carbon atoms,

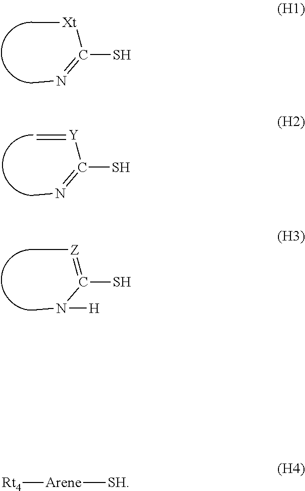

### Optional Glycidvl Hydroxy Benzoic Acid Condensate Additive

In one aspect of the positive chemically amplified photoresist composition described herein which contain the inventive covalent PAG comprising a N-(carbonylcarbamido) aryl sulfate PAG, these compositions may further comprise at least one glycidyl hydroxy benzoic acid condensate additive comprising one or more compounds having structure (GHBC-1), or more specifically structures (GHBC-2) or (GHBC-3). Compositions comprising these N-(carbonylcarbamido) aryl sulfate additives are useful in thick film resist formulations employed in applications where it is needed to form metallic lines with good adhesion during metal electroplating operations.

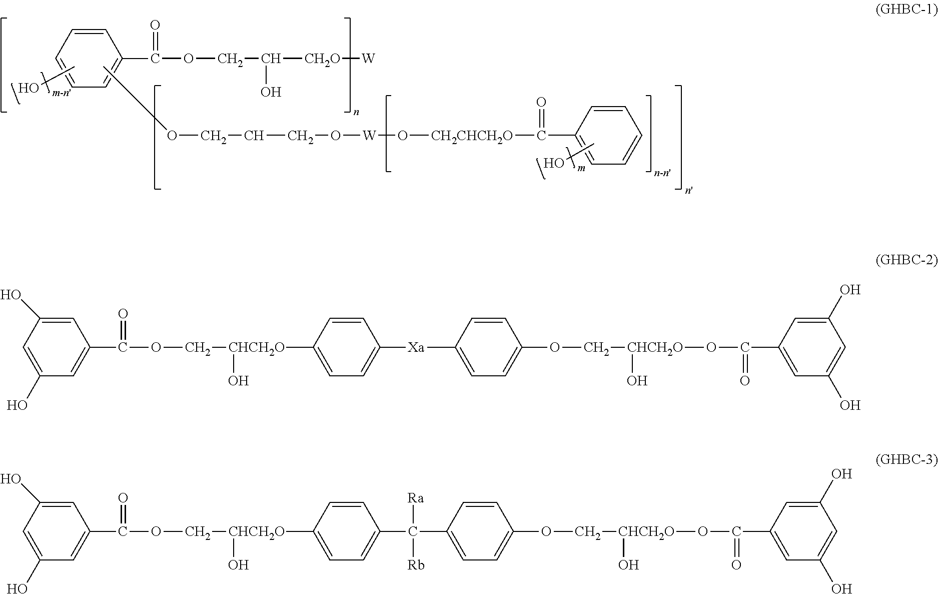

In structure (GHBC-1), W is an organic moiety having a molecular weight of 600 or less, wherein W forms an ether bond with the oxygen to which it is bound, m is an integer from 1 to 3 and n is an integer from 1 to 4. Where further, when m is 1, n is 3 or 4, and when m is 2 or 3, n is an integer from 1 to 4, n′ is 0 or 1. These additive may be at least one compound having structure (GHBC -2), wherein, Xa is selected from the group consisting of a direct valence bond, alkylene, —SO₂—, —C(═O)— and —O—. More specifically these additives may be ones having structure (GHBC-3), wherein Ra and Rb are independently a C-1 to C-5 alkyl moiety, or a C-2 to C-5-alkylene-O-alkyl moiety. In one aspect of this embodiment Ra and Rb are both methyl. Other additives of this type in the positive chemically amplified photoresist as described in US2022/0019141.

### Photobleaching Dye

The optional photobleachable dye component is one which absorbs the same radiation as photoacid generator (in this instance the inventive covalent PAG comprising a N-(carbonylcarbamido) aryl sulfate, described herein), is the same radiation as the one used to expose coated films arising from the negative and positive chemically amplified photoresist compositions described herein. Further, the bleachable dye has approximately similar or lower rate of photobleaching than the rate of photolysis of the photoacid generator component. The rate of bleaching of the dye is preferably not significantly higher than the photolysis of the photoacid generator. Preferably no more than 95% dye bleaching should occur at the resist dose to clear. The clearing dose of the resist is defined as the minimum exposure dose required to clear the resist in a large clear area for a given process.

Preferably the bleachable dye is a diazonaphthoquinone sulfonate ester of a polyhydroxy compound or monohydroxy phenolic compound, which can be prepared by esterification of 1,2-napthoquinonediazide-5-sulfonyl chloride and/or 1,2-naphthoquinonediazide-4-sulfonyl chloride with a phenolic compound or a polyhydroxy compound having 2-7 phenolic moieties, and in the presence of basic catalyst. Diazonaphthoquinones as photoactive compounds and their synthesis are well known to the skilled artisan. These compounds, which comprise a component of the present invention, are preferably substituted diazonaphthoquinone dyes, which are conventionally used in the art in positive photoresist formulations. Such sensitizing compounds are disclosed, for example, in U.S. Pat. Nos. 2,797,213, 3,106,465, 3,148,983, 3,130,047, 3,201,329, 3,785,825 and 3,802,885. Useful photobleachable dyes include, but are not limited to, the sulfonic acid esters made by condensing phenolic compounds such as hydroxy benzophenones, oligomeric phenols, phenols and their derivatives, novolaks and multisubstituted-multihydroxyphenyl alkanes with naphthoquinone-(1,2)-diazide-5-sulfonyl chloride and/or naphtho-quinone-(1,2)-diazide-4-sulfonyl chlorides. In one embodiment of the bleachable dye, monohydroxy phenols such as cumylphenol are preferred. In another embodiment of the bleachable dye, the number of the phenolic moieties per one molecule of the polyhydroxy compound used as a backbone of bleachable dye is in the range of 2-7, and more preferably in the range of 3-5.

Some representative examples of polyhydroxy compounds are: (a) Polyhydroxybenzophenones such as 2,3,4-trihydroxybenzophenone, 2,4,4′-trihydroxybenzophenone, 2,4,6-trihydroxybenzophenone, 2,3,4-trihydroxy-2′-methylbenzophenone, 2,3,4,4′-tetrahydroxybenzophenone, 2,2′,4,4′-tetrahydroxybenzophenone, 2,4,6,3′,4′-pentahydroxybenzophenone, 2,3,4,2′,4′-pentahydroxy-benzophenone, 2,3,4,2′,5′-pentahydroxybenzophenone, 2,4,6,3′,4′,5′-hexahydroxybenzophenone, and 2,3,4,3′,4′,5′-hexahydroxybenzophenone; (b) Polyhydroxyphenylalkylketones such as 2,3,4-trihydroxyacetophenone, 2,3,4-trihydroxyphenylpentylketone, and 2,3,4-trihydroxyphenylhexylketone; (c) Bis(polyhydroxyphenyl)alkanes such as bis(2,3,4-trihydroxyphenyl)methane, bis(2,4-dihydroxyphenyl)methane, and bis(2,3,4-trihydroxyphenyl)propane; (d) Polyhydroxybenzoates such as propyl 3,4,5-trihydroxy-benzoate, phenyl 2,3,4-trihydroxybenzoate, and phenyl 3,4,5-trihydroxybenzoate; (e) Bis(polyhydroxybenzoyl)alkanes or bis(polyhydroxybenzoyl)aryls such as bis(2,3,4-trihydroxybenzoyl)methane, bis(3-acetyl-4,5,6-trihydroxyphenyl)methane, bis(2,3,4-trihydroxybenzoyl)benzene, and bis(2,4,6-trihydroxybenzoyl)benzene; (f) Alkylene di(polyhydroxybenzoates) such as ethyleneglycol-di(3,5-dihydroxybenzoate) and ethylene glycol di(3,4,5-trihydroxybenzoate); (g) Polyhydroxybiphenyls such as 2,3,4-biphenyltriol, 3,4,5-biphenyltriol, 3,5,3′,5′-biphenyltetrol, 2,4,2′,4′-biphenyltetrol, 2,4,6,3′,5′-biphenylpentol, 2,4,6,2′,4′,6′-biphenylhexol, and 2,3,4,2′,3′,4′-biphenylhexol; (h) Bis(polyhydroxy)sulfides such as 4,4′-thiobis(1,3-dihydroxy)benzene; (i) Bis(polyhydroxyphenyl)ethers such as 2,2′,4,4′-tetrahydroxydiphenyl ether; (j) Bis(polyhydroxyphenyl)sulfoxides such as 2,2′,4,4′-tetrahydroxydiphenylsulfoxide; (k) Bis(polyhydroxyphenyl)sulfones such as 2,2′,4,4′-tetrahydroxydiphenylsulfone; (1) Polyhydroxytriphenylmethanes such as tris(4-hydroxyphenyl)methane, 4,4′,4″-trihydroxy-3,5,3′,5′-tetramethyltriphenylmethane, 4,4′,3″,4″-tetrahydroxy-3,5,3′,5′-tetramethyltriphenylmethane, 4,4′,2″,3″,4″-pentahydroxy-3,5,3′,5′-tetramethyltriphenylmethane, 2,3,4,2′,3′,4′-hexahydroxy-5,5′-diacetyltriphenylmethane, 2,3,4,2′,3′,4′,3″,4″-octahydroxy-5,5-diacetyltriphenylmethane, and 2,4,6,2′,4′,6′-hexahydroxy-5,5′-dipropionyltriphenylmethane; (m) Polyhydroxy-spirobi-indanes such as 3,3,3′,3′-tetramethyl-1,1′-spirobi-indane-5,6,5′,6′-tetrol, 3,3,3′,3′-tetramethyl-1,1′-spirobi-indane-5,6,7,6′6′,7′-hexol, and 3,3,3′3′-tetramethyl-1,1′-spirobi-indane-4,5,6,4′,5′,6′-hexol; (n) Polyhydroxyphthalides such as 3,3-bis(3,4-dihydroxyphenyl)phthalide, 3,3-bis(2,3,4-trihydroxyphenyl)phthalide, and 3′,4′,5′,6′-tetrahydroxyspiro(phthalide-3,9′-xanthene); (o) Polyhydroxy compounds described in JP No. 4-253058 such as alpha, alpha′ alpha″-tris (4-hydroxyphenyl)-1,3,5-triisopropylbenzene, alpha, alpha′, alpha″-tris(3,5-dimethyl-4-hydroxyphenyl)-1,3,5-triisopropylbenzene, alpha, alpha′, alpha″-tris (3,5-diethyl-4-hydroxyphenyl)-1,3,5-triisopropylbenzene, alpha, alpha′, alpha″-tris (3,5-di-n-propyl-4-hydroxyphenyl)-1,3,5-tri-isopropylbenzene, alpha, alpha′,alpha″-tris(3,5-diisopropyl-4-hydroxyphenyl)-1,3,5-triisopropylbenz ene, alpha, alpha′, alpha″-tris(3,5-di-n-butyl-4-hydroxyphenyl)-1,3,5-triisopropylbenzene, alpha, alpha′, alpha″-tris(3-methyl-4-hydroxyphenyl)-1,3,5-triisopropyl-benzene, alpha, alpha′, alpha″-tris(3-methoxy-4-hydroxyphenyl)-1,3,5-triisopropylbenzene, alpha, alpha′, alpha″-tris(2,4-dihydroxyphenyl)-1,3,5-triisopropylbenzene, 2,4,6-tris(3,5-dimethyl-4-hydroxyphenylthiomethyl)mesitylene, 1-[alpha-methyl-alpha-(4″-hydroxyphenyl)ethyl]-4-[alpha, alpha′-bis(4″-hydroxyphenyl)ethyl]benzene, 1-[alpha-methyl-alpha-(4′-hydroxyphenyl)ethyl]-3-[alpha, alpha′-bis(4″-hydroxy-phenyl)ethyl]benzene, 1-[alpha-methyl-alpha-(3′, 5′-dimethyl-4′-hydroxyphenyl)ethyl]benzene, 1-[alpha-methyl-alpha-(3′-methoxy-4′-hydroxyphenyl)ethyl]-4-[alpha′,alpha′-bis(3′-methoxy-4′-hydroxyphenyl)ethyl]benzene, and 1-[alpha-methyl-alpha-(2′,4′-dihydroxyphenyl)ethyl]-4-[alpha,alpha′-bis(4′-hydroxyphenyl)ethyl]benzene.

Other examples of naphthoquinonediazide photoactive compounds include condensation products of novolak resins with a naphthoquinonediazide sulfonyl chloride. These condensation products (also called capped novolaks) may be used instead of o-quinonediazide esters of polyhydroxy compounds or used in combination therewith. Numerous U.S. Patents describe such capped novolaks, U.S. Pat. No. 5,225,311 is one such example. Mixtures of various naphthoquinone-diazide compounds may also be used. The bleachable dye may be present in the novel photoresist composition at levels up to 15 weight % of total solids, preferably ranging from about 0.1% to about 10% of total solids, more preferably from about 0.30 to about 5% of total solids, and even more preferably from about 0.35% to about 2.5% of total solids.

### An Optional Dye Component

In a particular embodiment of negative chemically amplified photoresist, an additional dye component may be present to assist the formation of undercut profiles which are desirable in lift off application where the negative photoresist pattern is removed after using it to affect selective metal deposition using Vacuum Deposition induced by e-beam (EBPVD), chemical vapor deposition (CVD) or sputtering. In this instance, a dye component having a molar attenuation coefficient at 365 nm ranging from about 1.74×10⁴ to about 0.94×10⁴ mole⁻¹ L cm-1 is preferred (as measured in PGMEA). These I-line sensitive dye (a.k.a. 365 nm) may include ones such as Sudan Orange G; Martins Yellow; Dye O-PM ester; 2,3′,4,4′-tetramethylhydroxybenzophenone, 9-anthracene methanol; phenoxymethyl anthracene; 9,10-diphenylanthracene; substituted phenanthracenes and substituted biphenyls and the like.

In another embodiment such optional dye component may be ones which are aqueous base soluble and have structure (1od), wherein m1 and m2, independently, are 1 to 3, in another aspect of this embodiment m1 and m2 are both 2, in another aspect of this embodiment m1 is 1 and m2 is 3. Other examples of dyes having structure (1od), are ones where m1 and m2 may range from 0 to 3, with the proviso that at least one of either m1 or m2 is not 0. An example of a specific aqueous soluble dye is one having structure (2od).

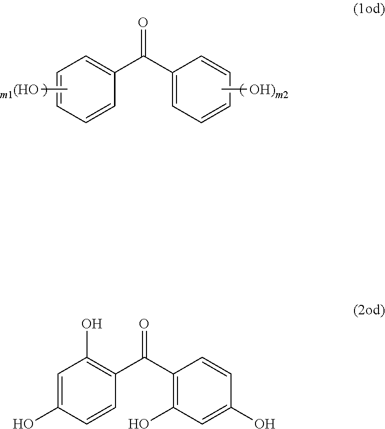

### An Optional Latent Electrophilic Crosslinking Component

For negative chemically amplified photoresists a latent cationic crosslinking agent which may be part of the photoresist component 2a) may be used in conjunction with a resin component which is soluble in 0.26 N aqueous TMAH. One type of such crosslinkers are monomeric latent electrophiles such as those which can form benzylic cations upon the action of a photoacid, such as compounds comprising a benzylic alcohol, benzylic acetates moieties or mixtures of these. Examples of such materials are ones that contain two or more benzylic acetate or benzyl alcohol moieties such as for example 1,4-phenylenebis(methylene) diacetate or DMLL-POP.

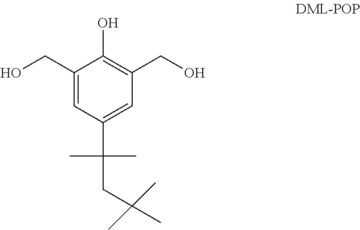 [SMILES: CC(C)(C)CC(C)(C)c1cc(CO)c(O)c(CO)c1]

Other latent electrophilic crosslinker which can form reactive cations upon action of a photoacid are etherified aminoplast, such as those based on melamines where this etherified aminoplast crosslinking agent comprises an organic oligomer or polymer that provides a carbonium ion upon and serves to crosslink said resin component in the presence of an acid generated by radiation, preferably imaging radiation. This renders said resin insoluble in an alkaline medium, in the exposed areas. Such crosslinking agents may be prepared from a variety of aminoplasts in combination with a compound or low molecular weight polymer containing a plurality of hydroxyl, carboxyl, amide, or imide groups. Preferred amino oligomers or polymers are aminoplasts obtained by the reaction of an amine, such as urea, melamine, or glycolurea with an aldehyde, such as formaldehyde. Such suitable aminoplasts include urea-formaldehyde, melamine-formaldehyde, benzoguanamine-formaldehyde, and glycoluril-formaldehyde resins, and combinations of any of these. A particularly preferred aminoplast is hexa(methoxymethyl) melamine oligomer.

In another embodiment of any of the above aspects said crosslinking agent, comprises etherified melamines selected from ones having structure (1cc), oligomers formed by (1cc) or mixtures of these; wherein R_{1c}, is a C-1 to C-4 alkyl, H or represents a moiety of structure (1cca), wherein [symbol] represents the attachment point of moiety (1cca) to structure (1cc), wherein R_{1cca} is a C-1 to C-4 alkyl, H or represents another moiety of structure (1cca).

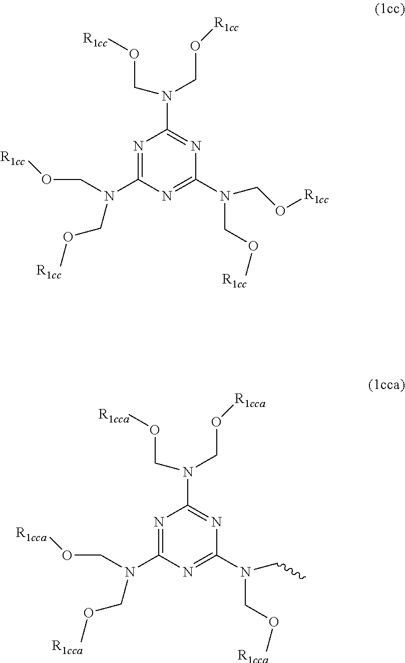

In another embodiment of any of the above aspects of this invention said solid component c), said crosslinking agent, comprises etherified melamines selected from ones having structure (2cc), oligomers formed by (2cc) or mixtures of these; wherein R_{2cc} is methyl, H or represents a moiety of structure (2cca), wherein [symbol] represents the attachment point of moiety (2cca) to structure (2cc), wherein R₂ccₐ is methyl, H or represents another moiety of structure (2cca).

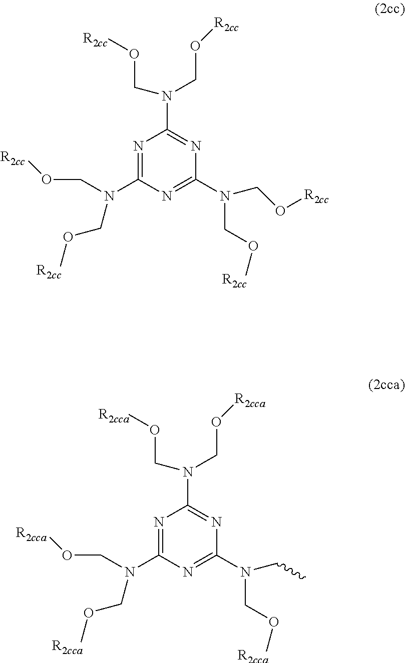

### Optional Different Type of PAG Component

The positive and negative chemically amplified photoresist formulation, as described herein may optionally, further comprise at least one additional different type of PAG which is not the one of the inventive PAG or a mixtures the inventive PAGs of different structures as described herein.

This optional different type of PAG may be any other type of material sensitive to radiation such as UV radiation (e.g. broadband, i-line, g-line, 248 nm 193 nm and EUV), which upon exposure to this radiation release an acid (a.k.a. photo-acid) which can cleave acid labile group such as tert-alkyl esters, or acetals relating a base solubilizing group in a resin employed in a positive chemically amplified photoresist making these exposed regions base soluble generating a positive image, or alternatively in negative chemically amplified photoresist cleave a group to generate a carbocation which can react with the photoresist resin to crosslink a base soluble resin making the expose resin insoluble in the exposed region generating a negative image. This photo-acid may be a sulfonic acid, HCl, HBr, HAsF₆, and the like. It includes as non-limiting examples onium salts and other photosensitive compounds as known in the art that can photochemically generate strong acids such that do not contain a fluoroalkyl or perfluoroalkyl groups such as alkylsulfonic acids, arylsulfonic acids, HAsF₆, HSbF₆, HBF₄, p-toluenesulfonic acid, and cyclopentadiene penta-substituted with electron withdrawing groups such as cyclopenta-1,3-diene-1,2,3,4,5-pentacarbonitrile. Other photoacid generators include trichloromethyl and tribromomethyl compounds and photosensitive derivative of trichoromethyl heterocyclic compounds or tribromomethyl heterocyclic compounds which can generate a hydrogen halide such as HBr or HCl. The PAG may be an aromatic imide N-oxysulfonate derivative of an aryl or alkyl sulfonic acid, an aromatic sulfonium salt of an organic sulfonic acid, a trihalotriazine derivative or a mixture thereof.

FIG. 4 shows non-limiting examples of optional other photoacid generators which generate sulfonic, and other strong acids.

FIG. 5 shows non-limiting examples of optional trichloromethyl or tribromo photoacid generators which generate HCl or HBr. In one aspect of this embodiment, it has structure (P) wherein Rip is a fluoroalkyl moiety and R₂p is H, an alkyl, an oxyalkyl, a thioalkyl, or an aryl moiety. Alternatively, this PAG may have structure (PA) wherein R₃p is an alkyl or an aryl moiety and R₄p is H, an alkyl, an oxyalkyl, a thioalkyl, or an aryl moiety.

This Optional Different Type of PAG component, as described herein, may range from about 0.1 wt. % to about 2 wt. % of total wt. % solids.

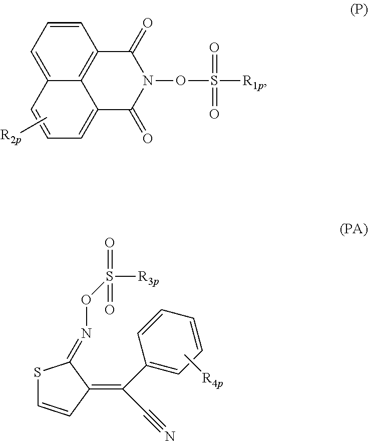

### Optional Surfactant Component

The positive and negative chemically amplified photoresist formulation, as described herein may optionally, further comprise at least one optional surface leveling agent, such as one or more surfactants. In this embodiment, there is no particular restriction with regard to the surfactant, and the examples of it include a polyoxyethylene alkyl ether such as polyoxyethylene lauryl ether, polyoxyethylene stearyl ether, polyoxyethylene cetyl ether, and polyoxyethylene olein ether; a polyoxyethylene alkylaryl ether such as polyoxyethylene octylphenol ether and polyoxyethylene nonylphenol ether; a polyoxyethylene polyoxypropylene block copolymer; a sorbitane fatty acid ester such as sorbitane monolaurate, sorbitane monopalmitate, and sorbitane monostearate; a nonionic surfactant of a polyoxyethylene sorbitane fatty acid ester such as polyoxyethylene sorbitane monolaurate, polyoxyethylene sorbitane monopalmitate, polyoxyethylene sorbitane monostearate, polyethylene sorbitane trioleate, and polyoxyethylene sorbitane tristearate; a fluorinated surfactant such as F-Top EF301, EF303, and EF352 (manufactured by Jemco Inc.), Megafac F171, F172, F173, R08, R30, R90, and R94 (manufactured by Dainippon Ink & Chemicals, Inc.), Florad FC-430, FC-431, FC-4430, and FC-4432 (manufactured by Sumitomo 3M Ltd.), Asahi Guard AG710, Surflon S-381, S-382, S-386, SC101, SC102, SC103, SC104, SC105, SC106, Surfinol E1004, KH-10, KH-20, KH-30, and KH-40 (manufactured by Asahi Glass Co., Ltd.); an organosiloxane polymer such as KP-341, X-70-092, and X-70-093 (manufactured by Shin-Etsu Chemical Co., Ltd.); and an acrylic acid or a methacrylic acid polymer such as Polyflow No. 75 and No. 95 (manufactured by Kyoeisha Chemical Co. Ltd.). When a surfactant is present in one embodiment it ranges from about 0.01 wt. % to about 0.3 wt. % of total solids.

### Optional Base Additive Components

In the positive and negative chemically amplified photoresist formulations, described herein, an optional component which may be added is a base component to moderate acid diffusion in the exposed region of the photoresist resulting from the photo-acid. This base component may be any base component sufficiently basic to neutralize the photo-acid. This base component can include, but is not limited to, a basic material or combination of materials such as an amine compound or a mixture of amine compounds having a boiling point above 100° C., at atmospheric pressure, and a pKₐ of at least 1. Such acid quenchers include, but are not limited to, amine compounds having structures (BIa), (BIb), (BIc), (BId), (BIe), (BIf), (BIg), (BIh), (BIi) (BIj), (BIk) and (BIl) or a mixture of compounds from this group; wherein R_{b1} is C-1 to C-20 saturated alkyl chain or a C-2 to C-20 unsaturated alkyl chain; R_{b2}, R_{b3}, R_{b4}, R_{b5}, R_{b6}, R_{b7}, R_{b8}, R_{b9}, R_{b10}, R_{b11}, R_{b12} and R_{b13}, are independently selected from the group of H, and a C-1 to C-20 alkyl.

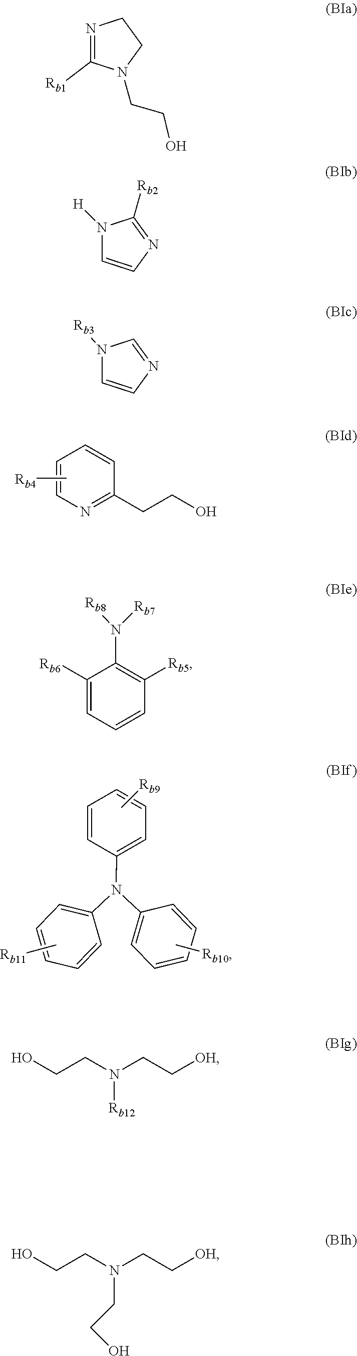

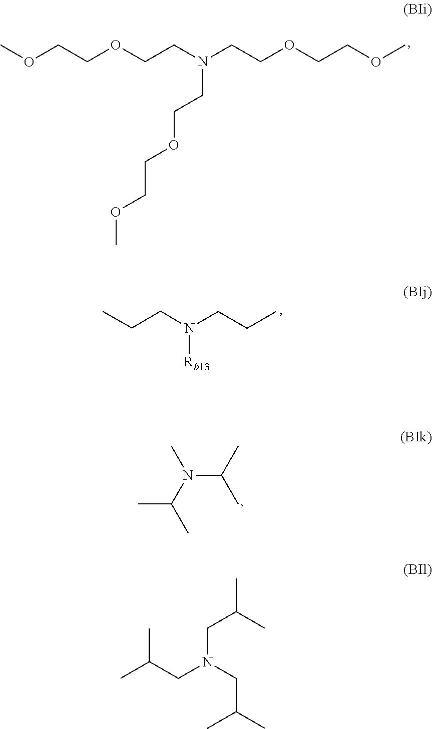

This base additive component can be chosen from, but is not limited to, a basic material or combination of materials which are tetraalkylammonium or trialkylammonium salts of a dicarboxylic acid or mixtures of these. Specific non limiting examples are mono(tetraalkyl ammonium) of dicarboxylic acid, di(tetraalkyl ammonium) salts of dicarboxylic acid, mono(trialkyl ammonium) of dicarboxylic acid, or di(trialkyl ammonium) salts of dicarboxylic acid. Non-limiting examples of suitable dicarboxylic acid for these salts are oxalic acid, maleic acid, malonic acid, fumaric acid, phthalic acid, and the like. Structure (BIma) to (BImd) gives a general structure for such materials wherein Rqa to Rqd are independently a C-4 to C-8 alkyl group, Rqe is a valence bond, an arylene moiety, a C-1 to C-4 alkylene moiety, an alkenyl moiety(-C(Rqf)═C(Rqg)-, wherein Rqf and Rqg are independently H or a C-1 to C-4 alkyl). Structure (BIme) gives a specific example of such a material.

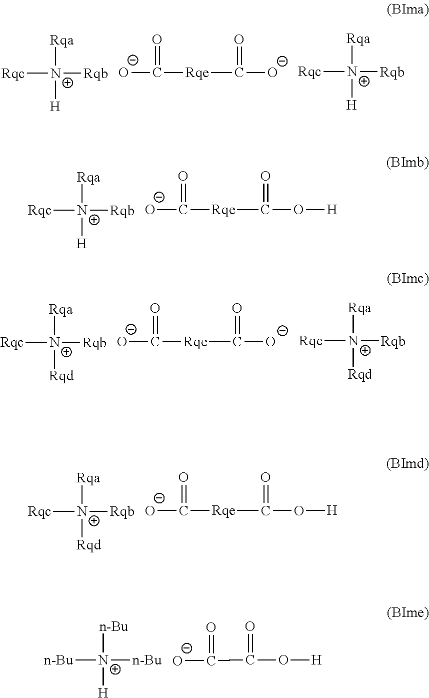

This base additive component, if present, ranges from about 0.0001 wt. % to about 0.020 wt. % of total solids.

### Optional Sensitizer Component

In the positive and negative chemically amplified photoresist formulations, described herein, an optional component in these photoresists which are designed for i-line or broadband irradiation are sensitizers to this radiation which may be used to induce more efficient photoacid generation from the inventive covalent PAG comprising a N-(carbonylcarbamido) aryl sulfate, described herein, when particular derivatives of these are not directly sensitive to i-line or broadband radiation, but which has been sensitized to this radiation with such photosensitizers that extend the effective wavelength and/or energy range. Such photosensitizers may be, without limitation, substituted and unsubstituted anthracenes, substituted and unsubstituted phenothiazines, substituted and unsubstituted perylenes, substituted and unsubstituted pyrenes, and aromatic carbonyl compounds, such as benzophenone and thioxanthone, fluorene, carbazole, indole, benzocarbazole, acridone chlorpromazine, equivalents thereof or combinations of any of the foregoing. FIG. 6 shows the structures and maximum wavelength absorbance of specific representative sensitizers.

### Organic Spin Coating Solvent

Organic spin coating solvents suitable for dissolving the above-described positive or negative chemically amplified photoresist compositions include a glycol ether derivative such as ethyl cellosolve, methyl cellosolve, propylene glycol monomethyl ether (PGME), diethylene glycol monomethyl ether, diethylene glycol monoethyl ether, dipropylene glycol dimethyl ether, propylene glycol n-propyl ether, or diethylene glycol dimethyl ether; a glycol ether ester derivative such as ethyl cellosolve acetate, methyl cellosolve acetate, or propylene glycol monomethyl ether acetate (PGMEA); carboxylates such as ethyl acetate, n-butyl acetate and amyl acetate; carboxylates of di-basic acids such as diethyloxylate and diethylmalonate; dicarboxylates of glycols such as ethylene glycol diacetate and propylene glycol diacetate; and hydroxy carboxylates such as methyl lactate, ethyl lactate (EL), ethyl glycolate, and ethyl-3-hydroxy propionate; a ketone ester such as methyl pyruvate or ethyl pyruvate; an alkoxycarboxylic acid ester such as methyl 3-methoxypropionate, ethyl 3-ethoxypropionate, ethyl 2-hydroxy-2-methylpropionate, or methylethoxypropionate; a ketone derivative such as methyl ethyl ketone, acetyl acetone, cyclopentanone, cyclohexanone or 2-heptanone; a ketone ether derivative such as diacetone alcohol methyl ether; a ketone alcohol derivative such as acetol or diacetone alcohol; a ketal or acetal like 1,3 dioxalane and diethoxypropane; lactones such as butyrolactone; an amide derivative such as dimethylacetamide or dimethylformamide, anisole, and mixtures thereof.

### Description of Suitable Polymer Comprising One of More Acid Cleavable Groups for Use in Positive Chemically Amplified Photoresist

These materials are ones which, when the acid cleavable group becomes cleaved catalytically by photogenerated acid in a region of a photoresist film, render this exposed photoresist film soluble at room temperature in an aqueous base developer such as 0.26 N aqueous tetramethyl ammonium hydroxide (TMAH) and other similar basic aqueous developer. This solubility may either require a post-exposure bake prior to development to enable for high activation energy acid cleavable groups (e.g. tert-butyl) or may not require such a post-exposure bake in the case of low activation energy acid cleavable groups (e.g., acetal).

For instance, said polymer comprising one or more acid cleavable groups said polymer may be a polymer comprising one or more (meth)acrylate repeat units and further comprising one or more repeat units with at least one acid cleavable group.

Another example of a polymer comprising one or more acid cleavable groups is a reaction product formed in the absence of an acid catalyst between (i) a Novolak polymer, (ii) a polymer comprising substituted or unsubstituted hydroxystyrene and acrylate, methacrylate, or a mixture of acrylate and methacrylate, the acrylate and/or methacrylate being protected by an acid labile group that requires a high activation energy for deblocking, and (iii) a compound selected from a vinyl ether and an unsubstituted or substituted, unsaturated heteroalicyclic compound. This type of polymer may be used alone, as described in US2009/00811589, or in combination with at least one other polymer comprising repeat units derived from 4-hydroxystyrene, repeat units derived from an acetal protected 4-hydroxystyrene, and a repeat unit derived from a (meth)acrylic acid protected with a high energy protecting group and other such reaction products as described in US2020-0183278.

Another example of a polymer comprising one or more acid cleavable groups is a (meth)acrylate copolymer comprising a (meth)acrylic acid derived repeat unit, whose carboxylic acid is functionalized with an acid labile group, and repeat units derived from at least one of styrene and benzyl (meth)acrylate,

Another example of a polymer comprising one or more acid cleavable groups is the one which comprises at least one (meth)acrylate copolymer comprising a (meth)acrylic acid derived repeat unit, whose carboxylic acid is functionalized with an acid labile group, and repeat units derived from at least one of styrene and benzyl (meth)acrylate, such materials may be used in conjunction with an aqueous base soluble component such as the base soluble components, described herein. In some embodiments, this copolymer is combined with at least one Novolak resin component which is soluble in 0.26 aqueous TMAH.

An example of a specific positive chemically amplified photoresist composition comprising the inventive photoacid generator, as described herein, is one which comprises a reaction product formed in the absence of an acid catalyst between (i) a Novolak polymer, (ii) a polymer comprising substituted or unsubstituted hydroxystyrene and acrylate, methacrylate or a mixture of acrylate and methacrylate, the acrylate and/or methacrylate being protected by an acid labile group that requires a high activation energy for deblocking as described in US2022-0019141and which generally described as follows:
a) at least one inventive photoacid generator as described herein,
b) at least one Novolak polymer,
c) at least one acrylate polymer, comprising a component having structure (I),

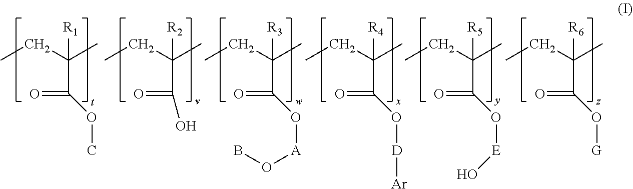

wherein R₁ to R₆ are, independently, —H, or —CH₃, A is a linear or branched C-2 to C-10 alkylene group, B is a C-1 to C-12 primary or secondary unsubstituted linear, branched, cyclic or alicyclic alkyl group, C is a C-1 to C-12 primary or secondary unsubstituted linear, branched, cyclic or alicyclic alkyl group, D is a linking group that is a direct valence bond, or a linear or branched C-2 to C-10 alkylene group, Ar is a substituted or unsubstituted aromatic group or heteroaromatic group, E is a linear or branched C-2 to C-10 alkylene group, G is an acid cleavable group, t is 0 mole % to about 40 mole %, v is 0 mole % to about 15 mole %, w is 0 mole % to about 45 mole %, x is 0 mole % to about 80 mole %, y is about 20 mole % to about 50 mole % and z is about 20 mole % to about 50 mole %, and further wherein the sum of t, v, w, x, y and z equals 100 mole %.

In one specific example of a positive chemically amplified photoresist composition which can employ the inventive to a positive chemically amplified photoresist composition comprising components a), b), c), d), and e), wherein component c) would comprise at last one of the inventive photoacid generators described herein:
a) at least one Diazonaphthoquinonesulfonate Photoactive Compound (DNQ-PAC),
b) at least one heterocyclic thiol having structure (7), (8) and/or (9),
c) at least one of the inventive photoacid generators,
d) at least one acrylic polymer comprising repeat units selected from ones having structure (1), (2), (3), (4), (5), and (6), where R₁, R₂, R₄, R₅, and R₆, individually, are selected from H or a C-1 to C-4 alkyl, R₇ is H or a C-1 to C-8 alkyl, R₈ is a C-1 to C-12 alkyl, R₉ is a C-2 to C-15 hydroxyalkylene(HO-alkylene-), R₁₀ is an acid cleavable group, and R₁ is a C-2 to C-15 alkyloxyalkylene (alkyl-O-alkylene-).

In one aspect of the inventive composition described herein said repeat units of said acrylate polymer are selected from the group consisting of repeat units having structure (1), (2), (3), (4), (5), and (6).

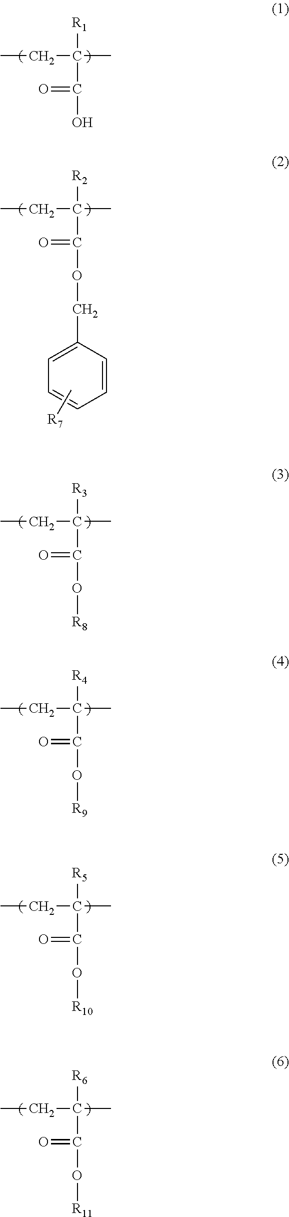

In another aspect of this inventive composition said repeat units of said acrylate polymer are selected from the group consisting of repeat units having structure (1), (2), (4), (5), and (6).

In any of the aspects of the inventive composition described herein said acrylate polymer is one wherein:
Structure (1) ranges from about 0 to about 35 mole %,
Structure (2) ranges from about 5 to about 55 mole %,
Structure (3) ranges from about 0 to about 30 mole %,
Structure (4) ranges from about 15 to about 55 mole %,
Structure (5) ranges from about 10 to about 40 mole %, and
Structure (6) ranges from about 0 to about 25 mole %,
   In a preferred embodiment, said acrylate polymer is one wherein:
Structure (1) ranges from about 5 to about 20 mole %,
Structure (2) ranges from about 5 to about 25 mole %,
Structure (3) ranges from about 0 to about 30 mole %,
Structure (4) ranges from about 15 to about 55 mole %,
Structure (5) ranges from about 20 to about 40 mole %, and
Structure (6) ranges from about 5 to about 25 mole %.

In another aspect of this inventive composition said acrylate polymer is one whose repeat units are the ones having structures (1), (2a), (4a), (5), and (6a) wherein n and n′ are the numbers of methylene spacer moieties and range, independently, from 1 to 4, R₁, R₂, R₄, R₅, and R₆, individually, are selected from a C-1 to C-4 alkyl, R₉, and Ru, are independently selected from H or a C-1 to C-4 alkyl, and R_{11″}, is a C-1 to C-4 alkyl. In one aspect of this embodiment, structure (1) ranges from about 5 to about 20 mole %, structure (2a) ranges from about 5 to about 25 mole %, structure (4a) ranges from about 15 to about 55 mole %, structure (5) ranges from about 20 to about 40 mole %, and structure (6a) ranges from about 5 to about 25 mole %.

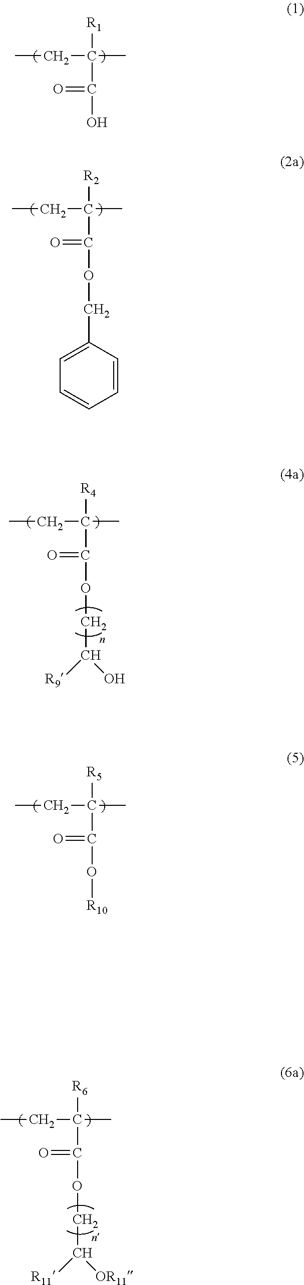

In any of inventive compositions described herein said acrylate polymer component is one wherein for said repeat unit of structure (5), R₁₀ is an acid cleavable group selected from the group consisting of a t-butyl group, a tetrahydropyran-2-yl group, a tetrahydrofuran-2-yl group, a 4-methoxytetrahydropyran-4-yl group, a 1-ethoxyethyl group, a 1-butoxyethyl group, a 1-propoxyethyl group, a 3-oxocyclohexyl group, a 2-methyl-2-adamantyl group, a 2-ethyl-2-adamantyl group, a 8-methyl-8-tricyclo[5.2.1.0 2,6]decyl group, a 1,2,7,7-tetramethyl-2-norbornyl group, a 2-acetoxymenthyl group, a 2-hydroxymethyl group a 1-methyl-1-cyclohexylethyl group, a 4-methyl-2-oxotetrahydro-2H-pyran-4-yl group, a 2,3-dimethylbutan-2-yl group, a 2,3,3-trimethylbutan-2-yl group, a 1-methyl cyclopentyl group, a 1-ethyl cyclopentyl group, a 1-methyl cyclohexyl group, 1-ethyl cyclohexyl group, a 1,2,3,3-tetramethylbicyclo[2.2.1]heptan-2-yl group, a 2-ethyl-1,3,3-trimethylbicyclo[2.2.1]heptan-2-yl group, a 2,6,6-trimethylbicyclo[3.1.1]heptan-2-yl group, a 2,3-dimethylpentan-3-yl group, or a 3-ethyl-2-methylpentan-3-yl group.

In one aspect of the inventive compositions described herein said acrylate polymer is one whose repeat units are the ones having structures (1), (2b), (4b), (5a), and (6b). In another aspect of this embodiment, structure (1a) ranges from about 5 to about 20 mole %, structure (2b) ranges from about 5 to about 25 mole %, structure (4b) ranges from about 15 to about 55 mole %, structure (5a) ranges from about 20 to about 40 mole %, and (6b) ranges from about 5 to about 25 mole %.

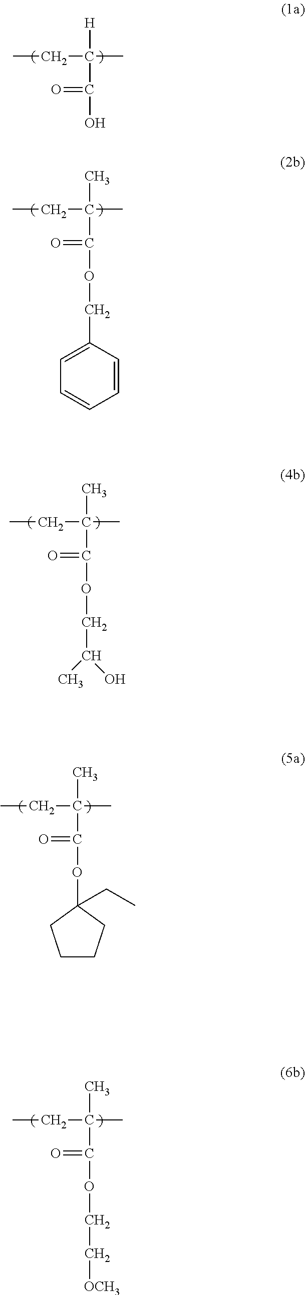

In the embodiments of the inventive composition described herein said acrylic polymer is one comprising repeat units selected from ones having structure (1), (2), (3), (4), (5), and (6), wherein (1) ranges from about 0 to about 35 mole %, (2) ranges from about 5 to about 55 mole %, (3) ranges from about 0 to about 30 mole %, (4) ranges from about 15 to about 55 mole %, (5) ranges from about 10 to about 40 mole %, and (6) ranges from about 0 to about 25 mole %, additionally other types of (meth)acrylic repeat unit and/or styrenic repeat units may be present. In this embodiment, said acrylic polymer may comprise at least one styrenic repeat units selected from the ones having the structure (14), where R₁₄ is chosen from H or CH₃ and R₁₄, and R₁₄ₑ can be the same or different, and are chosen from H, OH, ORₚ, O—C(═O)—ORₚ, or O—C(═O)—C(═O)—ORₚ wherein Rp is an acid labile group having the same scope as described herein for the acid labile group R₁₀. Preferably, in this embodiment, the polymer comprises at least one styrenic repeat unit selected from the ones having the structure (14), where R₁₄ is chosen from H, or CH₃, and R₁₄, and Rie can be the same or different, and are chosen from H, OH, OCOOC(CH₃)₃, or OCOCOO(CH₃)₃ A specific non limiting Rp is a tertiary alkyl having at least one beta-hydrogen capable of elimination to form an alkene upon acidolytic cleavage by H+(e.g., tert-butyl). Further, in this embodiment, said acrylic polymer may comprise at least one (meth)acrylate of a lactone moiety which is either a single cyclic lactone, or a lactone moiety comprised within an alicyclic alkyl. Said lactone moiety may be either a single cyclic lactone, or a lactone moiety comprised within an alicyclic alkyl. More specific examples of such (meth)acrylate of a lactone moiety are shown in structure (15), wherein R₁₅ is chosen from H or CH₃ and m is 1 or 2. In one aspect of this embodiment said acrylic polymer additionally comprises both a styrenic repeat unit of structure (1) and (meth)acrylate repeat unit of structure (15).

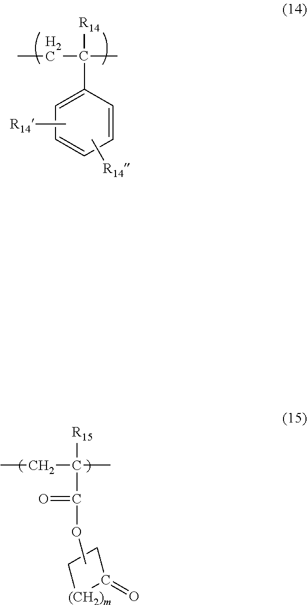

For the inventive composition described herein component d) of said acrylate polymer may, without limitation, have a weight average molecular weight in the range from 800 Daltons to 30,000 Daltons. Further exemplary weight average molecular weights of the structure may, without limitation, range from 1,500 Daltons to 20,000 Daltons. Still further exemplary weight average molecular weights of the structure may, without limitation, range from 2,500 Daltons to 20,000 Daltons. Molecular weight can be determined by gel permeation chromatography using a universal calibration method, calibrated to polystyrene standards.

Another aspect of said inventive negative chemically amplified photoresist composition is one wherein said thiol derivative component is present and is selected from the group consisting of thiol derivatives having the structures (H1), (H2) (H3), or (H4), wherein. in said structure (H1), Xt is selected from the group consisting of N(Rt₃), C(Rt₁)(Rt₂), O, S, Se, and Te;
in said structure (H2), Y is selected from the group consisting of C(Rt₃) and N;
in said structure (H3), Z is selected from the group consisting of C(Rt₃) and N; and
in said structure (H4), where Arene is selected from phenyl, a substituted phenyl, an unsubstituted polycyclic arene moiety and a substituted polycyclic arene moiety, Rt₁, Rt₂, and Rt₃ are independently selected from the group consisting of H, a substituted alkyl group having 1 to 8 carbon atoms, an unsubstituted alkyl group having 1 to 8 carbon atoms, a substituted alkenyl group having 2 to 8 carbon atoms, an unsubstituted alkenyl group having 2 to 8 carbon atoms, a substituted alkynyl group having 2 to 8 carbon atoms, an unsubstituted alkynyl group having 2 to 8 carbon atoms, a substituted aromatic group having 6 to 20 carbon atoms, a substituted heteroaromatic group having 3 to 20 carbon atoms, an unsubstituted aromatic group having 6 to 20 carbon atoms and an unsubstituted heteroaromatic group having 3 to 20 carbon atoms,
Rt₄ is independently selected from the group consisting of H, OH, a substituted alkyl group having 1 to 8 carbon atoms, an unsubstituted alkyl group having 1 to 8 carbon atoms, a substituted alkenyl group having 2 to 8 carbon atoms, an unsubstituted alkenyl group having 2 to 8 carbon atoms, a substituted alkynyl group having 2 to 8 carbon atoms, an unsubstituted alkynyl group having 2 to 8 carbon atoms, a substituted aromatic group having 6 to 20 carbon atoms, a substituted heteroaromatic group having 3 to 20 carbon atoms, an unsubstituted aromatic group having 6 to 20 carbon atoms and an unsubstituted heteroaromatic group having 3 to 20 carbon atoms,

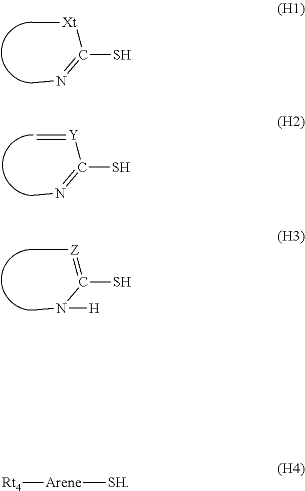

In another aspect of said inventive negative chemically amplified photoresist composition, as described above, which is one either containing, or not said thiol derivative, said photoresist resin soluble in aqueous base is at least one phenolic film-forming polymeric binder resin having ring bonded hydroxyl groups, which is either selected from a Novolak resin, a hydroxystyrene copolymer, or mixtures thereof, which are soluble in 0.26 N aqueous TMAH. In this aspect this composition further comprises a crosslinking agent that forms a carbonium ion upon exposure to acid photogenerated by the PAG and which comprises an etherified aminoplast polymer or oligomer; and also comprises an organic spin casting solvent.

In another aspect of said inventive negative chemically amplified photoresist composition, as described above, either comprising, or not, said thiol derivative, said photoresist resin comprises a novolak derived from a substituted phenol selected from ortho-cresol; meta-cresol; para-cresol; 2,4-xylenol; 2,5-xylenol; 3,4-xylenol, 3,5-xylenol, thymol and mixtures thereof, which have been condensed with an aldehyde; a poly(vinyl phenol), and also comprises a poly(vinyl phenol) copolymer. In one aspect of this embodiment said aldehyde is formaldehyde.

In another aspect of said inventive negative chemically amplified photoresist composition, as described above, either comprising, or not, said thiol derivative, said crosslinking component is present. In another aspect of this embodiment, said crosslinking component is an etherified aminoplast oligomer or a polymer obtained by the reaction of an amine with an aldehyde. In another aspect of this embodiment, said crosslinking component is an etherified aminoplast oligomer or polymer obtained by the reaction of an amine with an aldehyde and is a hexa(methoxymethyl) melamine. In another aspect of this embodiment, said crosslinking component is present, and is an etherified aminoplast oligomer or polymer obtained by the reaction of an amine with an aldehyde and is a dialkylol cresol. In another aspect of this embodiment said crosslinking component is present and is an etherified aminoplast oligomer or polymer obtained by the reaction of an amine with an aldehyde and is a dialkylol cresol which is a dialkylol para-cresol. In another aspect of this embodiment, said crosslinking component is present, and is an etherified aminoplast oligomer or polymer obtained by the reaction of an amine with an aldehyde and is a dialkylol cresol which is a dihydroxyalkyl-(tetra-alkyl)-phenol.

### Process of Forming an Image with a Positive Chemically Amplified Photoresist

Another aspect of this invention is a process of forming a positive image with a positive photoresist exposed to radiation, comprising step i) to v);
i) coating any one of the above described inventive positive chemically amplified photoresist on a substrate,
ii) baking said coated film to form a baked film,
iii) exposing regions of the baked film through a mask with radiation, forming exposed and unexposed regions,
iv) an optional post exposure baking step,
v) developing away with an aqueous base, said exposed region, forming a positive image on said substrate.

### Process of Forming an Image with a Negative Chemically Amplified Photoresist

Another aspect of this is invention is a process is a process of forming negative image with a negative photoresist by exposure to radiation, comprising step ia) to va)
ia) coating any one of the above described inventive negative chemically amplified photoresist on a substrate,
iia) baking said coated film to form a baked film,
iiia) exposing regions of the baked film through a mask with radiation, forming exposed and unexposed regions,
iva) an optional post exposure baking step,
va) developing away the unexposed regions forming a negative image on said substrate.

In one aspect of the above-described inventive compositions they consist essentially of described components, where the term “consist essentially of” entails that other components may be present that do not affect the performance of the material and are present only in a concentration totaling about 10 wt. % of the composition. In another aspect of these embodiments, these other components are present only in a concentration totaling about 5 wt. % of the composition. In a further aspect of these embodiments these other components are present only in a concentration totaling about 1 wt. %. In still another aspect of these embodiments these other components are present in a concentration totaling about 0.5 wt. %.

In another aspect of the above-described inventive compositions they consist of listed components which excludes the presence of other components.

Another aspect of this invention is the use of the covalent compound comprising an imide N-(carbonylcarbamido) aryl sulfate moiety wherein said compound is free of any fluoroalkyls, any perfluoroalkyls or mixtures of fluoroalkyls and perfluoroalkyls, as a photoacid generator, preferably in photoresist compositions.

### Chemicals and Characterization

All chemicals unless otherwise indicated were purchased from Sigma-Aldrich, Inc. (3050 Spruce St., St. Louis, MO 63103).

All synthetic experiments were carried out under N2 atmosphere. Lithographic experiments were carried out as described in the text.

An ASML i-line stepper PAS5500 was used to expose the resist films of the examples which was provided at 0.48 numerical aperture. The resist pattern profile cross sections were examined by means of scanning electron microscopy. A Hitachi S-4700 or Hitachi SU8030 SEM were used to evaluate resist profiles.

Thermogravimetric analysis (TGA) was carried out under constant nitrogen flow 10° C./min, using a TGA Discovery instrument, from room temperature to 600° C. The temperatures at which the weight loss is 1% and 5% were recorded.

¹H NMR and ¹³C spectra were obtained at room temperature on a Bruker spectrometer, using deuterated chloroform as a solvent, and calibrated at 7.26 ppm or 77.16 ppm, respectively.

A Thermo Fisher Scientific Vanquish Flex HPLC-MS/MS (Thermo Fisher Scientific QExactive Plus) equipped with a positive mode APCI ionization was used to analyze samples. A Hypersil Gold C18 column (150×2.1 mm, 1.9 μm particle size) was used for separation and kept at 30° C. Solvent A was water with 0.1% formic acid, and solvent B was acetonitrile with 0.1% formic acid.

Differential scanning calorimetry analysis was carried out on a DSC 2500 instrument, under nitrogen with a heating rate of 10° C./min. The melting point (Tm) was measured in first heating scan.

The new class of covalent PAG, free of any fluoroalkyls, any perfluoroalkyls or mixtures of fluoroalkyls and perfluoroalkyls, are ones comprising a N-(carbonylcarbamido)oxy aryl sulfonate was tested with the representative compound PAG 1 PAG which formulated and exposed successfully in both positive and negative chemically amplified photoresist (CAR) formulations.

The CAR formulations were spin coated on a substrate, soft baked on a hotplate, exposed with i-line stepper by a mask, then post-exposure-bake (PEB) and developed with aqueous alkaline solution. Finally, the wafers were rinsed with DI water and then spin dried to obtain photoresist patterns.

Scheme 1 shows the synthetic pathway used to make the 1,8-naphthalimide N-phenyl sulfate PAG, denoted as PAG 1, and described in more detail in Synthesis examples 1 to 3.

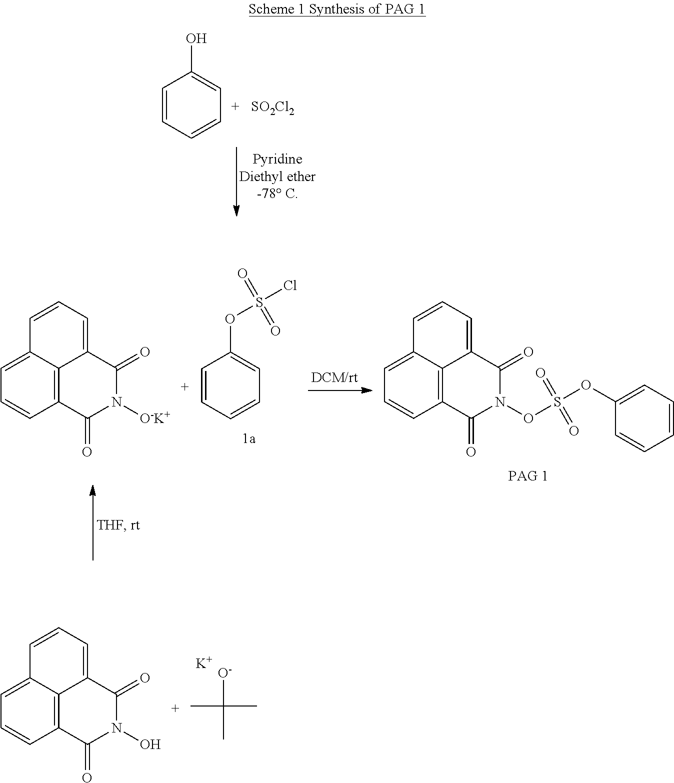

### Synthesis Example 1 Synthesis of N-Hydroxy-1,8-Naphthalimide Potassium Salt

N-hydroxy-1,8-naphthalimide potassium salt was prepared following a published procedure (US2006122408A1). N-Hydroxy-1,8-naphthalimide, (6.4 g; 30 mmol) and potassium t-butoxide (3.37 g; 30 mmol) were dispersed in dry tetrahydrofuran (200.00 m1) under inert atmosphere and stirred at room temperature overnight. Afterwards, the solvent was removed under vacuum to give N-hydroxy-1,8-naphthalimide potassium salt (7.5 g) as a dark red solid. The salt was used in the next step without further purification.

### Synthesis Example 2 Synthesis of Phenyl Chlorosulfate (La)

Phenyl chlorosulfate (1a) was prepared following a published procedure (J. Am. Chem. Soc. 2013, 135, 29, 10638-10641): Phenol (4.7 g, 50 mmol, 1 equiv.) was added to an oven-dried 250 mL round-bottom flask equipped with a large stir bar and evacuated and backfilled with argon. This process was repeated for three times. Afterwards, anhydrous diethyl ether (50 mL) and anhydrous pyridine (4 mL, 50 mmol, 1 equiv.) were added into the flask and the obtained solution was cooled to −78° C. In a separate oven-dried 100 mL round-bottom flask that was evacuated and backfilled with argon for three times, anhydrous diethyl ether (50 mL) was added and then the flask was placed into a dry ice/acetone bath. Sulfuryl chloride (4 m1, 50 mmol, 1 equiv.) was slowly added to the cooled diethyl ether and stirred for 30 minutes. Then, the obtained sulfuryl chloride solution was slowly transferred via canula to the vigorously stirred solution of phenol and pyridine at −78° C. After stirring for 2 h at −78° C., the reaction mixture was allowed to slowly warm to room temperature overnight. The crude reaction mixture was filtered through a pad of celite, and the reaction flask was washed with additional diethyl ether. The filtrate was concentrated, and the resulting residue was purified immediately on silica gel eluting with a solvent mixture ethyl acetate/heptane (3:97 (v/v)) to afford 7.5 g of phenyl chlorosulfate as a colorless oil (78% yield).

¹H NMR (500 MHz, CDCl₃, δ): 7.52-7.40 (m, 5H). ¹³C NMR (125 MHz, CDCl₃, δ): 150.0, 130.2, 128.8, 121.6.

### Synthesis Example 3 Synthesis of 1,8-naphthalimide N-phenyl sulfate (PAG 1)

N-hydroxy-1,8-naphthalimide potassium salt (7.5 g, 30 mmol, 1 equiv.) was dispersed in anhydrous dichloromethane (150 m1) under inert gas. Afterwards, phenyl chlorosulfate 1a (5.8 g, 30 mmol, 1 equiv.) was added dropwise to the externally cooled suspension. The mixture was stirred at room temperature for overnight. Dichloromethane was removed under vacuum, and the solid taken with ethyl acetate and water. The organic phase was washed with water, brine, separated, dried over anhydrous sodium sulfate, filtered off and concentrated. The resulting residue was purified on silica gel eluting with a solvent mixture ethyl acetate/heptane (1:2 (v/v)) to afford 7.2 g of 1,8-naphthalimide N-phenyl sulfate (PAG 1) as yellow solid (65% yield). ¹H NMR (500 MHz, CDCl₃, δ): 8.72-8.70 (d, 2H), 8.33-8.32 (d, 2H), 7.85-7.82 (t, 2H), 7.74-7.71 (d, 2H) 7.52-7.48 (t, 2H), 7.42-7.38 (t, 1H). ¹³C NMR (125 MHz, CDCl₃, δ): 159.5, 151.1, 135.7, 132.8, 132.1, 130.1, 128.1, 127.7, 127.5, 122.2, 121.8. MS (m/z): 370 [M]⁺. FIGS. 7 and 8 respectively shows the ¹H NMR and ¹³C NMR spectra of PAG 1. FIG. 20 shows the DSC of PAG 1.

Scheme 2 shows the general synthetic procedure for the preparation of phenyl chlorosulfates 1b-i.

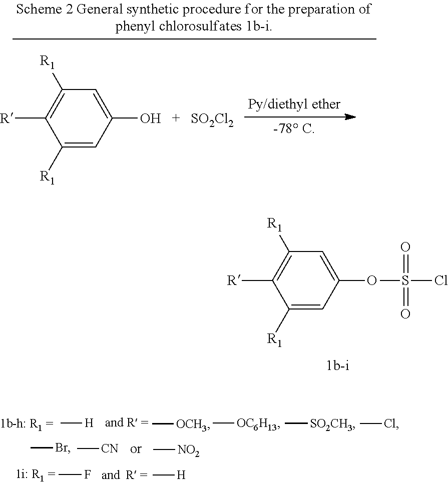

The synthesis of 1b-i follows the general procedure described for 1a in Synthesis example 2.

### Synthesis Example 4

4-Methoxyphenyl chlorosulfate (1b) purified via column chromatography on silica gel, n-heptane/ethyl acetate mixture as eluent; colorless liquid, yield 86%. ¹H NMR (500 MHz, CDCl₃, δ): 7.33-7.31 (2H, d); 6.97-6.95 (2H, d); 3.84 (3H, s).

### Synthesis Example 5

4-Hexyloxyphenyl chlorosulfate (1c) purified via column chromatography on silica gel, n-heptane/ethyl acetate mixture as eluent; colorless liquid, yield 75%. ¹H NMR (500 MHz, CDCl₃, δ): 7.31-7.29 (2H, d); 6.95-6.94 (2H, d); 3.98-3.95 (2H, t); 1.82-1.76 (2H, m); 1.49-1.43 (2H, m); 1.36-1.33 (4H, m); 0.93-0.90 (3H, t).

### Synthesis Example 6

4-Methylsulfonylphenyl chlorosulfate (1d) purified via column chromatography on silicagel, n-heptane/ethyl acetate mixture as eluent; white crystalline solid, yield 54%. ¹H NMR (500 MHz, CDCl₃, δ): 813-8.11 (2H, d); 7.64-7.62 (2H, d); 3.11 (3H, s).

### Synthesis Example 7

4-Chlorophenyl chlorosulfate (1e) purified via column chromatography on silica gel, n-heptane/ethyl acetate mixture as eluent; colorless liquid, yield 77%. ¹H NMR (500 MHz, CDCl₃, δ): 7.48-7.46 (2H, d); 7.36-7.35 (2H, d).

### Synthesis Example 8

4-Bromophenyl chlorosulfate (1f) purified via column chromatography on silica gel, n-heptane/ethyl acetate mixture as eluent; colorless liquid, yield 89%. ¹H NMR (500 MHz, CDCl₃, δ): 7.64-7.62 (2H, d); 7.30-7.28 (2H, d).

### Synthesis Example 9

4-Cyanophenyl chlorosulfate (1g) purified via column chromatography on silica gel, n-heptane/ethyl acetate mixture as eluent; white crystalline solid, yield 88%. ¹H NMR (500 MHz, CDCl₃, δ): 7.84-7.82 (2H, d); 7.56-7.54 (2H, d).

### Synthesis Example 10

4-Nitrophenyl chlorosulfate (1h) purified via column chromatography on silica gel, n-heptane/ethyl acetate mixture as eluent; yellow liquid, yield 76%. ¹H NMR (500 MHz, CDCl₃, 6): 8.40-8.38 (2H, d); 7.62-7.61 (2H, d).

### Synthesis Example 11

3,5-Difluorophenyl chlorosulfate (1i) purified via column chromatography on silica gel, n-heptane/ethyl acetate mixture as eluent; colorless liquid, yield 88%. ¹H NMR (500 MHz, CDCl₃, δ): 7.02-7.00 (2H, dd); 6.97-6.92 (1H, td). Synthetic pathway for N-hydroxy-4-(hex-1-yn-1-yl)-1,8-naphthalimide which is described in more detail in Synthesis Examples 12 and 13.

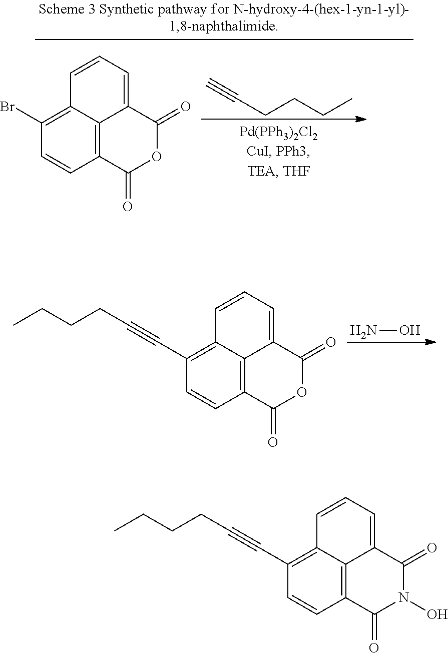

### Synthesis Example 12 Synthesis of 4-(hex-1-yn-1-yl)-1,8-naphthalic anhydride

A 1000-m1 4-neck round-bottom flask was charged with 4-bromo-1,8-naphthalic anhydride (75 g; 0.271 mol), triphenylphosphine (5.680 g; 21.655 mmol), triethylamine (79.230 m1; 568.448 mmol) and tetrahydrofuran (500 m1). The mixture was purged with argon for 1 hour. To this mixture copper iodide (1.547 g; 8.121 mmol) and bis(triphenylphosphine)palladium (II) dichloride (1.900 g; 2.707 mmol) were added. The mixture was heated to reflux and 1-hexyne (40.584 m1; 353.250 mmol) in tetrahydrofuran (80.000 m1) was added dropwise. After the full addition of 1-hexyne, the mixture was kept under reflux for 2 days. The mixture was allowed to cool down to room temperature and 4 mL water added. THF was removed under reduced pressure, the solid taken with DCM and washed with water. The organic phase was separated and dried over magnesium sulfate and concentrated under vacuum. The brown solid was recrystallized twice from acetonitrile to give 4-(hex-1-yn-1-yl)-1,8-naphthalic anhydride as yellow crystals (yield 66%). ¹H-NMR (500 MHz, CDCl₃, δ): 8.73-8.71 (d, 1H); 8.65-8.63 (d, 1H); 8.53-8.52 (d, 1H); 7.87-7.84 (m, 2H); 2.65-2.63 (t, 2H); 1.76-1.70 (m, 2H); 1.61-1.55 (m, 2H); 1.03-1.00 (t, 3H).

### Synthesis Example 13 Synthesis of N-hydroxy-4-(hex-1-yn-1-yl)-1,8-naphthalimide

4-(hex-1-yn-1-yl)-1,8-naphthalic anhydride (24.662 g; 0.089 mol) and hydroxylamine hydrochloride (6.158 g; 0.089 mol) in isopropanol (90.000 m1) were stirred at room temperature over 30 min. Triethylamine (12.351 m1; 0.089 mol) was slowly added to the above solution, and the mixture was stirred at 95° C. for 1 hour. After completion of the reaction, isopropanol and triethylamine were evaporated, the solid was washed with water, dried at 60° C. for 24 h and 90° C. for 24 h in a vacuum oven. Further recrystallization from ethanol gave N-hydroxy-4-(hex-1-yn-1-yl)-1,8-naphthalimide as yellow crystals (yield 92%). ¹H-NMR (500 MHz, DMSO-d₆, δ): 10.77 (s, 1H); 8.60-8.58 (d, 1H); 8.54-8.53 (d, 1H); 8.42-8.40 (d, 1H); 7.96-7.94 (t, 1H); 7.90-7.89 (d, 1H); 2.68-2.65 (t, 2H); 1.68-1.64 (m, 2H); 1.54-1.49 (m, 2H); 0.98-0.95 (t, 3H).

### General Synthetic Procedure for the Preparation of PFAs-Free PAG 2-11

Various covalent PAGs 2 to 4 have been synthesized using the commercially available N-hydroxy-1,8-naphthalimide sodium salt that can react with phenyl chlorosulfate derivates in acetonitrile at reflux temperature. For the structures noted as PAGs 5 to 11, a two-step one-pot procedure was employed, where in the first step the sodium salt was prepared in situ from sodium hydride and the corresponding N-hydroxy-4-(hex-1-yn-1-yl)-1,8-naphthalimide, followed in the second step by the reaction of N-hydroxy-4-(hex-1-yn-1-yl)-1,8-naphthalimide sodium salt with the corresponding phenyl chlorosulfate derivate as shown in Scheme 4.

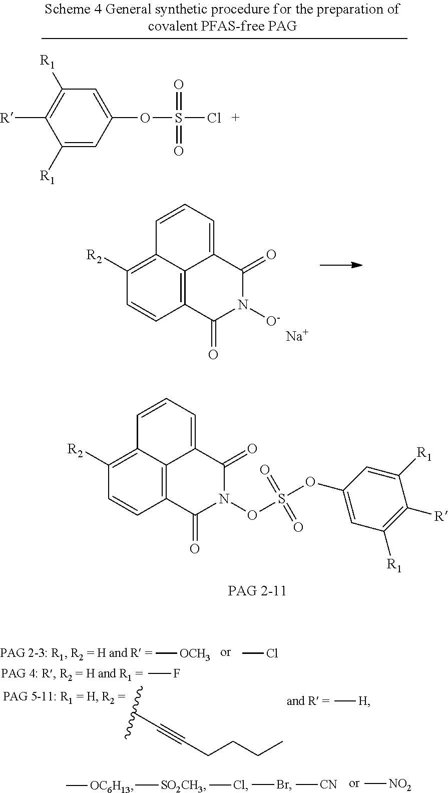

### Synthesis Example 14 Synthesis of 1,8-naphthalimide N-(4-methoxyphenyl) sulfate (PAG 2)

The synthesis of PAG 3 and PAG 4 follows the general procedure described herein for PAG 2: In a 100-mL round bottom flask equipped with magnetic stirring bar, N-hydroxy-1,8-naphthalimide sodium salt (7.05 g, 30 mmol, 1 equiv.) was dispersed in anhydrous acetonitrile (50 m1) under inert gas. Afterwards, 4-methoxyphenyl chlorosulfate 2a (6.68 g, 30 mmol, 1 equiv.) was added dropwise to the externally cooled suspension. The mixture was stirred at reflux overnight. The mixture was allowed to cool down to room temperature, and water was added to quench the reaction. Acetonitrile was removed under vacuum, and the residue taken with ethyl acetate and water. The organic phase was washed with water, brine, separated, dried over anhydrous sodium sulfate, filtered off and concentrated. The resulting residue was purified on silica gel eluting with n-heptane/ethyl mixture (2:1 (v/v)) to afford 8.6 g of 1,8-naphthalimide N-(4-methoxyphenyl) sulfate (PAG 2) as white crystalline solid (yield 72%). ¹H NMR (500 MHz, CDCl₃, δ): 8.71-8.69 (2H, d); 8.33-8.31 (2H, d); 7.85-7.82 (2H, t); 7.67-7.65 (2H, d); 6.98-6.96 (2H, d); 3.84 (3H, s). FIG. 9 shows the ¹H NMR of PAG 2.

### Synthesis Example 15

1,8-naphthalimide N-(4-chlorophenyl) sulfate (PAG 3). purified via column chromatography on silica gel, n-heptane/ethyl acetate mixture as eluent; white crystalline solid, yield 71%. ¹H NMR (500 MHz, CDCl₃, δ): 8.71-8.69 (2H, d); 8.34-8.32 (2H, d); 7.85-7.82 (2H, t); 7.70-7.68 (2H, d); 7.47-7.45 (2H, d). FIG. 10 shows the ¹H NMR of PAG 3.

### Synthesis Example 16

1,8-naphthalimide N-(3,5-difluorophenyl) sulfate (PAG 4). purified via column chromatography on silica gel, n-heptane/ethyl acetate mixture as eluent; white crystalline solid, yield 82%. ¹H NMR (500 MHz, CDCl₃, δ): 8.71-8.69 (2H, d); 8.34-8.33 (2H, d); 7.86-7.83 (2H, t); 7.37-7.35 (2H, d); 6.91-6.88 (1H, t). FIG. 11 shows the ¹H NMR of PAG 4.

### Synthesis Example 17 Synthesis of 4-(hexyn-1-yl)-1,8-naphthalimide N-(4-hexyloxyphenyl) sulfate (PAG 5)

The synthesis of PAG 6-11 follows the general procedure described herein for PAG 5:Sodium hydride, 60% dispersion in mineral oil (409.080 mg; 10.228 mmol) was introduced in a oven-dried flask under argon atmosphere, and suspended in anhydrous THF (50 mL) and cooled down to 0° C. In a separate oven-dried flask, N-hydroxy-4-(hex-1-yn-1-yl)-1,8-naphthalimide (3.000 g; 10.228 mmol) was dissolved in dry THF (50 mL) under argon atmosphere and cannulated to the NaH solution. The reaction was stirred at room temperature overnight. Phenyl chlorosulfate 1a (1.970 g; 10.228 mmol) was introduced dropwise in the cooled THF solution of N-hydroxy-4-(hex-1-yn-1-yl)-1,8-naphthalimide sodium salt formed in situ. The reaction mixture was brought stepwise to reflux and stirred overnight. The mixture was allowed to cool to room temperature and then water was added to quench the reaction. THF was removed under reduced pressure. The solid was taken with ethyl acetate, washed with water. The organic phase was separated, dried over magnesium sulfate, filtered off through a Celite pad, and concentrated. The crude mixture was subjected to purification via column chromatography on silica gel using n-heptane/dichloromethane mixture (1:1 (v/v)) to afford 3.3 g of 4-(hexyn-1-yl)-1,8-naphthalimide N-phenyl sulfate (PAG 5) as yellow solid (yield 71%). ¹H-NMR (500 MHz, CDCl₃, δ): 8.76-8.74 (d, 1H); 8.72-8.70 (d, 1H); 8.60-8.59 (d, 1H); 7.87-7.84 (m, 2H); 7.73-7.71 (d, 2H); 7.51-7.48 (t, 2H); 7.41-7.39 (t, 1H); 2.66-2.63 (t, 2H); 1.76-1.71 (m, 2H); 1.60-1.57 (m, 2H); 1.03-1.00 (t, 3H). ¹³C-NMR (125 MHz, CDCl₃, 6): 159.5; 159.2; 151.2; 134.3; 133.0; 132.3; 132.0; 131.0; 130.7; 130.1; 128.1; 127.7; 127.6; 122.4; 121.8; 120.7; 103.2; 77.8; 30.7; 22.3; 19.8; 13.8. FIGS. 12 and 13 respectively show the ¹H NMR and ¹³C NMR spectra of PAG 5. FIG. 21 shows the DSC for PAG 5.

### Synthesis Example 18

4-(hexyn-1-yl)-1,8-naphthalimide N-(4-hexyloxyphenyl) sulfate (PAG 6). purified via column chromatography on silica gel, n-heptane/ethyl acetate mixture as eluent; pale-yellow crystalline solid, yield 62%. ¹H NMR (500 MHz, CDCl₃, δ): 8.76-8.74 (1H, d); 8.72-8.70 (1H, d); 8.60-8.59 (1H, d); 7.87-7.84 (2H, t); 7.64-7.62 (2H, d); 6.96-6.94 (2H, d); 3.98-3.96 (2H, t); 2.66-2.63 (2H, t); 1.82-1.76 (2H, m); 1.75-1.71 (2H, m); 1.61-1.55 (2H, m); 1.50-1.44 (2H, m); 1.37-1.33 (4H, m); 1.03-1.00 (3H, t); 0.93-0.90 (3H, t). FIG. 14 shows the ¹H NMR of PAG 6.

### Synthesis Example 19

4-(hexyn-1-yl)-1,8-naphthalimide N-(4-methylsulfonylphenyl) sulfate (PAG 7). purified via column chromatography on silica gel, n-heptane/ethyl acetate mixture as eluent; beige crystalline solid, yield 52%. ¹H NMR (500 MHz, CDCl₃, δ): 8.77-8.76 (1H, d); 8.71-8.69 (1H, d); 8.59-8.58 (1H, d); 8.12-8.09 (2H, t); 7.95-7.93 (2H, d); 7.89-7.85 (2H, t); 3.11 (3H, s); 2.66-2.63 (2H, t); 1.77-1.71 (2H, m); 1.60-1.54 (2H, m); 1.03-1.00 (3H, t). FIG. 15 shows the ¹H NMR of PAG 7.

### Synthesis Example 20

4-(hexyn-1-yl)-1,8-naphthalimide N-(4-chlorophenyl) sulfate (PAG 8). purified via column chromatography on silica gel, n-heptane/ethyl acetate mixture as eluent; pale-yellow crystalline solid, yield 65%. ¹H NMR (500 MHz, CDCl₃, δ): 8.76-8.75 (1H, d); 8.71-8.70 (1H, d); 8.60-8.58 (1H, d); 7.88-7.85 (2H, t); 7.70-7.67 (2H, m); 7.47-7.44 (2H, m); 2.66-2.63 (2H, t); 1.75-1.71 (2H, m); 1.60-1.55 (2H, m); 1.03-1.00 (3H, t). FIG. 16 shows the ¹H NMR of PAG 8.

### Synthesis Example 21

4-(hexyn-1-yl)-1,8-naphthalimide N-(4-bromophenyl) sulfate (PAG 9). purified via column chromatography on silica gel, n-heptane/ethyl acetate mixture as eluent; pale-yellow crystalline solid, yield 51%. ¹H NMR (500 MHz, CDCl₃, δ): 8.76-8.74 (1H, d); 8.71-8.69 (1H, d); 8.59-8.58 (1H, d); 7.87-7.84 (2H, t); 7.64-7.59 (4H, m); 2.66-2.63 (2H, t); 1.75-1.71 (2H, m); 1.60-1.54 (2H, m); 1.03-1.00 (3H, t). FIG. 17 shows the ¹H NMR of PAG 9.

### Synthesis Example 22

4-(hexyn-1-yl)-1,8-naphthalimide N-(4-cyanophenyl) sulfate (PAG 10). purified via column chromatography on silica gel, n-heptane/ethyl acetate mixture as eluent; pale-yellow crystalline solid, yield 62%. ¹H NMR (500 MHz, CDCl₃, δ): 8.78-8.76 (1H, d); 8.71-8.69 (1H, d); 8.59-8.58 (1H, d); 7.89-7.85 (4H, m); 7.83-7.81 (2H, m); 2.66-2.63 (2H, t); 1.77-1.71 (2H, m); 1.61-1.55 (2H, m); 1.03-1.00 (3H, t). FIG. 18 shows the ¹H NMR of PAG 10.

### Synthesis Example 23

4-(hexyn-1-yl)-1,8-naphthalimide N-(4-nitrophenyl) sulfate (PAG 11). purified via column chromatography on silica gel, n-heptane/ethyl acetate mixture as eluent; pale-yellow crystalline solid, yield 52%. ¹H NMR (500 MHz, CDCl₃, δ): 8.78-8.76 (1H, d); 8.71-8.69 (1H, d); 8.60-8.58 (1H, d); 8.40-8.38 (2H, d); 7.92-7.86 (4H, m); 2.66-2.63 (2H, t); 1.75-1.71 (2H, m); 1.60-1.54 (2H, m); 1.03-1.00 (3H, t). FIG. 19 shows the ¹H NMR of PAG 11.

Table 3 shows a comparison of the TGA and Tₘ data for PAG 1 PAG 5 and NIT PAG. This TGA data confirmed that the inventive PAG 1 and PAG 5 have a surprisingly better thermal stability than the conventional NIT PAG which contains a trifluoromethyl alkyl, showing that these novel PAGs not only eliminate the need for a fluorinated alkyl to generate a strong photoacid upon irradiation but also but at the same time confers to these inventive PAGs a much greater thermal stability. FIG. 22 shows a comparison of TGA of PAG 1, PAG 5 and NIT PAG.

TABLE 3
TGA for PAG 1 PAG 5

| Sample | Tₘ (°) | T_{1%} (°) | T_{5%} (°) |
|---|---|---|---|
| PAG 1 | 172.1 | 230 | 271 |
| PAG 5 | 118.3 | 251 | 257 |
| NIT | 211.5 | 190 | 219 |

### Photoresist Formulation Examples—

The components in the formulation examples not obtained from Sigma-Aldrich, Inc, NIT PAG (N-Hydroxynaphthalimide triflate) (CAS Registry Number: 85342-62-7 was obtained from the Heraeus (Dayton Ohio).

Alnovol SPN560 FAST is a meta-cresol/formaldehyde Novolak resin, sold under the name of ALNOVOL™ SPN 560/47MPAC fast as a 46.8% PGMEA solution by Allnex USA Inc. The average molecular weight of this Novolak is MW=7,232. The dissolution rate of this Novolak resin is 1,594 Å/s in AZ 326 MIF developer.

Alnovol SPN560 SLOW is a meta-cresol/formaldehyde Novolak resin, sold under the name of ALNOVOL™ SPN 560/47MPAC slow as a 47.4% PGMEA solution by Allnex USA Inc. The average molecular weight of this Novolak is MW=19,612. The dissolution rate of this Novolak resin is 633 Å/s in AZ 326 MIF developer.

DML-POP is a latent electrophilic crosslinker from HONSHU Chemical Industry Co., LTD with the chemical name 2-hydroxy5-(1,1,3,3-tetramethylbutyl)-1,3-benzenedimethanol

KF-353A (CAS No. is 68937-54-2) is an organosiloxane polymeric surfactant from Shin-Etsu (Tokyo 100-0005, Japan). It has the following general structure where Rsi is an organic pendant group which comprises segments of both polyethylene glycol and polypropylene glycol and m and n are the number of repeat units in this organosiloxane polymer.

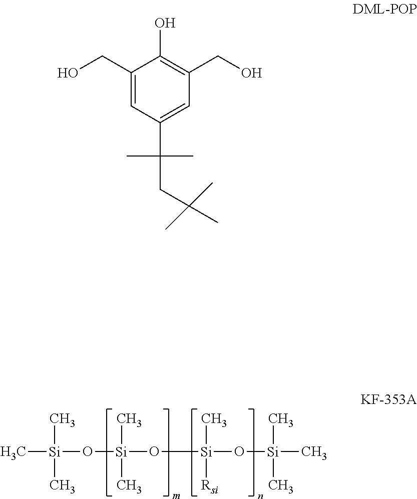

MOP-Triazine, is a photoacid generator from Sanwa Chemical Co., LTD and has the following structure:

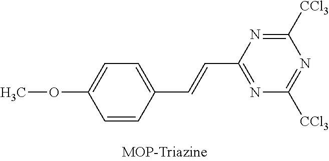

HMMM is 2,4,6-Tris[bis(methoxymethyl)amino]-1,3,5-triazine; purchased from TCI America.

PMT is 1-Phenyl-5-mercapto-1,2,3,4-tetrazole, purchased from TOYOBO CO., LTD.

TBA-Oxalate is Tributylammonium oxalate prepared according to US20190064662A1.

MTA is an additive, 1H-1,2,3-triazole-3-thiol, purchased from Sigma-Aldrich.

CKS-670F-EX is a bisphenol-A/m-cresol-formaldehyde Novolak copolymer sold under the name CKS-670F-EX. The average molecular weight is MW=9,500.

CYMEL 301 is a highly methylated melamine crosslinker supplied from Allnex Japan. It is a mixture comprising hexamethoxymethylmelamine and an oligomer resulting from the reaction of formaldehyde with melamine, where the hydroxy functionalities of this oligomer are etherified with methyl.

TMEEA is Tris[2-(2-methoxyethoxy)ethyl]amine purchased from Sigma-Aldrich.

PGMEA (1-Methoxy-2-propyl acetate), the solvent used for photoresist formulation examples was obtained from Lyondell Chemical Company.

1,3,4,6-Tetrakis(methoxymethyl)tetrahydroimidazo[4,5-d]imidazole-2,5(1H, 3H)-dione is obtained from Sigma-Aldrich.

NK-280 is a DNQ-PAC sold under this name by TOYO GOSEL, LTD.

AE6 Polymer is an acrylic polymer which was made according to “Acrylic Polymer Synthesis Example 9” in WO2021/094350.

PA-298 is a photoacid generator obtained from Heraeus, having the chemical structure below.

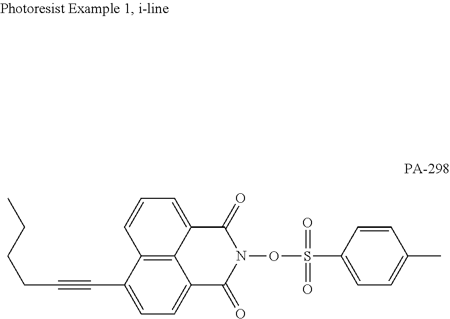 [SMILES: CCCCC#Cc1ccc2c3c(cccc13)C(=O)N(OS(=O)(=O)c1ccc(C)cc1)C2=O]

A CAR composition was made by dissolving 46.8% PGMEA solution of Alnovol SPN560 FAST (4.85 grams), 47.4% PGMEA solution of Alnovol SPN560 SLOW (4.79 grams), 36.38% PGMEA solution of CKS-670F-EX (12.43 grams), 56.85% PGMEA solution of CYMEL 301 (1.88 grams), 0.25 grams of DML-POP, 0.28 grams of PAG 1, 10.0% PGMEA solution of KF-353A surfactant (0.05 grams) and PGMEA 0.47 grams. Thus, a photoresist composition with a solid content of 42.65% by weight was prepared.

The photoresist composition was spin-coated on a silicon wafer substrate, soft-baked at 110° C./180 sec to obtain a film with 10.0 μm thickness. Next, through a pattern mask for measuring resolution, the coated film was exposed by an ASML i-line stepper (NA=0.48, δ=0.55), followed by post-exposure-bake (PEB) of 120° C./60 sec, and then developed with AZ® 626MIF developer (2.38% TMAH, tetramethylammonium hydroxide aqueous solution) for 50 sec with 3 puddles (3×50 sec). The dose-to-print of 5.0 μm L/S (line/space) resolved at 700 mJ/cm².

### Photoresist Example 2, i-line

A CAR composition was made by dissolving 46.8% PGMEA solution of Alnovol SPN560 FAST (7.96 grams), 47.4% PGMEA solution of Alnovol SPN560 SLOW (5.24 grams), 55.9% PGMEA solution of AE6 Polymer (7.41 grams), 0.0168 grams of MOP-Triazine, 0.0068 grams of MTA, 0.0078 grams of TBA-Oxalate, 0.1121 grams of PAG 1, 10.0% PGMEA solution of KF-353A surfactant (0.063 grams) and PGMEA 4.19 grams. Thus, a photoresist composition with a solid content of 42.00% by weight was prepared.

The photoresist composition was spin-coated on a silicon wafer substrate, soft-baked at 120° C./180 sec to obtain a film with 10.0 μm thickness. Next, through a pattern mask for measuring resolution, the coated film was exposed by an ASML i-line stepper (NA=0.48, δ=0.55), followed by post-exposure-bake (PEB) of 100° C./60 sec, and then developed with AZ® 626MIF developer (2.38% TMAH, tetramethylammonium hydroxide aqueous solution) for 60 sec with 2 puddles (2×60 sec). The dose-to-print of 5.0 μm L/S (line/space) resolved at 520 mJ/cm².

### Photoresist Example 3, i-line

A CAR composition was made by dissolving 46.8% PGMEA solution of Alnovol SPN560 FAST (9.07 grams), 47.4% PGMEA solution of Alnovol SPN560 SLOW (8.96 grams), 1.00 grams of 1,3,4,6-Tetrakis(methoxymethyl)tetrahydroimidazo[4,5-d]imidazole-2,5(1H, 3H)-dione, 0.23 grams of DML-POP, 0.25 grams of PAG 1, 9.5% PGMEA solution of TMEEA (0.26 grams), 10.0% PGMEA solution of KF-353A surfactant (0.05 grams) and PGMEA 5.18 grams. Thus, a photoresist composition with a solid content of 40.00% by weight was prepared.

The photoresist composition was spin-coated on a silicon wafer substrate, soft-baked at 110° C./180 sec to obtain a film with 10.0 μm thickness. Next, through a pattern mask for measuring resolution, the coated film was exposed by an ASML i-line stepper (NA=0.48, δ=0.55), followed by post-exposure-bake (PEB) of 120° C./60 sec, and then developed with AZ® 626MIF developer (2.38% TMAH, tetramethylammonium hydroxide aqueous solution) for 50 sec with 3 puddles (3×50 sec). The dose-to-print of 5.0 μm L/S (line/space) resolved at 350 mJ/cm².

### Photoresist Example 4, i-line

A CAR composition was made by dissolving 46.8% PGMEA solution of Alnovol SPN560 FAST (15.8 grams), 47.4% PGMEA solution of Alnovol SPN560 SLOW (10.4 grams), 55.9% PGMEA solution of AE6 Polymer (9.27 grams), 0.0109 grams of MTA, 0.2173 grams of PAG 1, 0.3622 grams of DNQ PAC NK280, 10.0% PGMEA solution of KF-353A surfactant (0.1087 grams) and PGMEA 13.83 grams. Thus, a photoresist composition with a solid content of 36.22% by weight was prepared.

The photoresist composition was spin-coated on a silicon wafer substrate, soft-baked at 120° C./120 sec to obtain a film with 8.0 μm thickness. Next, through a pattern mask for measuring resolution, the coated film was exposed by an ASML i-line stepper (NA=0.48, δ=0.55), followed by post-exposure-bake (PEB) of 90° C./60 sec, and then developed with AZ© 626MIF developer (2.38% TMAH, tetramethylammonium hydroxide aqueous solution) for 60 sec with 2 puddles (2×60 sec). The dose-to-print of 2.0 μm L/S (line/space) resolved at 350 mJ/cm².

### Photoresist Example 5, i-line

A CAR composition was made by dissolving 46.8% PGMEA solution of Alnovol SPN560 FAST (3.88 grams), 46.9% PGMEA solution of Alnovol SPN560 SLOW (3.87 grams), 36.38% PGMEA solution of CKS-670F-EX (9.98 grams), 56.85% PGMEA solution of CYMEL 301 (1.50 grams), 0.20 grams of DML-POP, 0.21 grams of PAG 5, 10.0% PGMEA solution of KF-353A surfactant (0.04 grams) and PGMEA 0.31 grams. Thus, a photoresist composition with a solid content of 42.65% by weight was prepared.

The photoresist composition was spin-coated on a silicon wafer substrate, soft-baked at 110° C./180 sec to obtain a film with 10.0 μm thickness. Next, through a pattern mask for measuring resolution, the coated film was exposed by an ASML i-line stepper (NA=0.48, δ=0.55), followed by post-exposure-bake (PEB) of 120° C./60 sec, and then developed with AZ® 300MIF developer (2.38% TMAH, tetramethylammonium hydroxide aqueous solution) for 50 sec with 3 puddles (3×50 sec). The dose-to-print of 5.0 μm L/S (line/space) resolved at 750 mJ/cm².

### Photoresist Example 6, i-line

A CAR composition was made by dissolving 46.8% PGMEA solution of Alnovol SPN560 FAST (6.34 grams), 47.4% PGMEA solution of Alnovol SPN560 SLOW (4.05 grams), 55.9% PGMEA solution of AE6 Polymer (4.42 grams), 0.0047 grams of MTA, 0.157 grams of PAG 1 PAG, 10.0% PGMEA solution of KF-353A surfactant (0.047 grams) and PGMEA 4.99 grams. Thus, a photoresist composition with a solid content of 39.2% by weight was prepared.

The photoresist composition was spin-coated on a silicon wafer substrate, soft-baked at 120° C./180 sec to obtain a film with 12.0 μm thickness. Next, through a pattern mask for measuring resolution, the coated film was exposed by an ASML i-line stepper (NA=0.48, δ=0.55), followed by post-exposure-bake (PEB) of 100° C./60 sec, and then developed with AZ© 300MIF developer (2.38% TMAH, tetramethylammonium hydroxide aqueous solution) for 60 sec with 2 puddles (2×60 sec). The dose-to-print of 4.0 μm L/S (line/space) resolved at 140 mJ/cm².

### Photoresist Example 7, i-line

A CAR composition was made by dissolving 46.8% PGMEA solution of Alnovol SPN560 FAST (7.04 grams), 47.4% PGMEA solution of Alnovol SPN560 SLOW (4.50 grams), 55.9% PGMEA solution of AE6 Polymer (4.01 grams), 0.0047 grams of MTA, 0.1568 grams of PAG 5, 10.0% PGMEA solution of KF-353A surfactant (0.047 grams) and PGMEA 4.23 grams. Thus, a photoresist composition with a solid content of 39.2% by weight was prepared.

The photoresist composition was spin-coated on a silicon wafer substrate, soft-baked at 120° C./180 sec to obtain a film with 12.0 μm thickness. Next, through a pattern mask for measuring resolution, the coated film was exposed by an ASML i-line stepper (NA=0.48, δ=0.55), followed by post-exposure-bake (PEB) of 100° C./60 sec, and then developed with AZ® 300MIF developer (2.38% TMAH, tetramethylammonium hydroxide aqueous solution) for 60 sec with 2 puddles (2×60 sec). The dose-to-print of 4.0 μm L/S (line/space) resolved at 260 mJ/cm².

### Photoresist Comparative Example 1, i-line

A CAR composition was made by dissolving 46.8% PGMEA solution of Alnovol SPN560 FAST (4.85 grams), 47.4% PGMEA solution of Alnovol SPN560 SLOW (4.79 grams), 36.38% PGMEA solution of CKS-670F-EX (12.48 grams), 56.85% PGMEA solution of CYMEL 301 (1.88 grams), 0.25 grams of DML-POP, 0.27 grams of NIT PAG, 10.0% PGMEA solution of KF-353A surfactant (0.05 grams) and PGMEA 0.44 grams. Thus, a photoresist composition with a solid content of 42.65% by weight was prepared.

The photoresist composition was spin-coated on a silicon wafer substrate, soft-baked at 110° C./180 sec to obtain a film with 10.0 μm thickness. Next, through a pattern mask for measuring resolution, the coated film was exposed by an ASML i-line stepper (NA=0.48, δ=0.55), followed by post-exposure-bake (PEB) of 120° C./60 sec, and then developed with AZ© 626MIF developer (2.38% TMAH, tetramethylammonium hydroxide aqueous solution) for 50 sec with 3 puddles (3×50 sec). The dose-to-print of 5.0 μm L/S (line/space) resolved at 450 mJ/cm².

### Photoresist Comparative Example 2, i-line

A CAR composition was made by dissolving 46.8% PGMEA solution of Alnovol SPN560 FAST (7.97 grams), 47.4% PGMEA solution of Alnovol SPN560 SLOW (5.24 grams), 55.9% PGMEA solution of AE6 Polymer (7.41 grams), 0.0168 grams of MOP-Triazine, 0.0068 grams of MTA, 0.0078 grams of TBA-Oxalate, 0.105 grams of NIT PAG, 10.0% PGMEA solution of KF-353A surfactant (0.063 grams) and PGMEA 4.18 grams. Thus, a photoresist composition with a solid content of 42.00% by weight was prepared.

The photoresist composition was spin-coated on a silicon wafer substrate, soft-baked at 120° C./180 sec to obtain a film with 10.0 μm thickness. Next, through a pattern mask for measuring resolution, the coated film was exposed by an ASML i-line stepper (NA=0.48, δ=0.55), followed by post-exposure-bake (PEB) of 100° C./60 sec, and then developed with AZ® 626MIF developer (2.38% TMAH, tetramethylammonium hydroxide aqueous solution) for 60 sec with 2 puddles (2×60 sec). The dose-to-print of 5.0 μm L/S (line/space) resolved at 140 mJ/cm².

### Photoresist Comparative Example 3, i-line

A CAR composition was made by dissolving 46.8% PGMEA solution of Alnovol SPN560 FAST (15.8 grams), 47.4% PGMEA solution of Alnovol SPN560 SLOW (10.4 grams), 55.9% PGMEA solution of AE6 Polymer (9.41 grams), 0.0109 grams of MTA, 0.0063 grams of TBA-Oxalate, 0.1358 grams of NIT PAG, 0.3622 grams of DNQ PAC NK280, 10.0% PGMEA solution of KF-353A surfactant (0.1087 grams) and PGMEA 13.77 grams. Thus, a photoresist composition with a solid content of 36.22% by weight was prepared.

The photoresist composition was spin-coated on a silicon wafer substrate, soft-baked at 120° C./120 sec to obtain a film with 8.0 μm thickness. Next, through a pattern mask for measuring resolution, the coated film was exposed by an ASML i-line stepper (NA=0.48, δ=0.55), followed by post-exposure-bake (PEB) of 90° C./60 sec, and then developed with AZ® 626MIF developer (2.38% TMAH, tetramethylammonium hydroxide aqueous solution) for 60 sec with 2 puddles (2×60 sec). The dose-to-print of 2.0 μm L/S (line/space) resolved at 150 mJ/cm².

### Photoresist Comparative Example 4, i-line

A CAR composition was made by dissolving 46.8% PGMEA solution of Alnovol SPN560 FAST (4.81 grams), 47.4% PGMEA solution of Alnovol SPN560 SLOW (4.75 grams), 36.38% PGMEA solution of CKS-670F-EX (12.37 grams), 56.85% PGMEA solution of CYMEL 301 (1.88 grams), 0.25 grams of DML-POP, 0.35 grams of Heraeus PA-298 PAG, 10.0% PGMEA solution of KF-353A surfactant (0.05 grams) and PGMEA 0.55 grams. Thus, a photoresist composition with a solid content of 42.65% by weight was prepared.

The photoresist composition was spin-coated on a silicon wafer substrate, soft-baked at 110° C./180 sec to obtain a film with 10.0 μm thickness. Next, through a pattern mask for measuring resolution, the coated film was exposed by an ASML i-line stepper (NA=0.48, δ=0.55), followed by post-exposure-bake (PEB) of 120° C./60 sec, and then developed with AZ© 626MIF developer (2.38% TMAH, tetramethylammonium hydroxide aqueous solution) for 50 sec with 3 puddles (3×50 sec). No lines smaller than 10 μm was printed below 700 mJ/cm². FIG. 23 shows Table 4 which compares the lithographic performance of Photoresist Examples 1 and 2 (Photoresist Ex. 1 and 2) to that of Photoresist comparative Examples 1 to 4 (Photoresist Comp. Ex 1 to 4).

Although the disclosed and claimed subject matter has been described and illustrated with a certain degree of particularity, it is understood that the disclosure has been made only by way of example, and that numerous changes in the conditions and order of steps can be resorted to by those skilled in the art without departing from the spirit and scope of the disclosed and claimed subject matter.

### Photoresist Comparative Example 5, i-line

A CAR composition was made by dissolving 46.8% PGMEA solution of Alnovol SPN560 FAST (17.10 grams), 47.4% PGMEA solution of Alnovol SPN560 SLOW (11.26 grams), 55.9% PGMEA solution of AE6 Polymer (10.18 grams), 0.0118 grams of MTA, 0.0069 grams of TBA-Oxalate, 0.147 grams of NIT PAG, 0.392 grams of DNQ PAC NK280, 10.0% PGMEA solution of KF-353A surfactant (0.118 grams) and PGMEA 10.78 grams. Thus, a photoresist composition with a solid content of 39.2% by weight was prepared.

The photoresist composition was spin-coated on a silicon wafer substrate, soft-baked at 120° C./180 sec to obtain a film with 12.0 μm thickness. Next, through a pattern mask for measuring resolution, the coated film was exposed by an ASML i-line stepper (NA=0.48, δ=0.55), followed by post-exposure-bake (PEB) of 100° C./60 sec, and then developed with AZ® 300MIF developer (2.38% TMAH, tetramethylammonium hydroxide aqueous solution) for 60 sec with 2 puddles (2×60 sec). The dose-to-print of 4.0 μm L/S (line/space) resolved at 140 mJ/cm².

FIG. 24 shows Table 5 in which a comparison is made of Photoresist example 6 formulated with the inventive PAG 1, and Photo resist Comparative Examples 5 formulated the fluoroalkyl based conventional PAG NIT which demonstrates that this inventive PAG gave an identical performance without the need of an undesirable perfluoroalkyl moieties.

### Photoresist Example 8, i-line

A CAR composition was made by dissolving 47.1% PGMEA solution of Alnovol SPN560 FAST (8.88 grams), 47.5% PGMEA solution of Alnovol SPN560 SLOW (2.09 grams), 56.0% PGMEA solution of AE6 Polymer (8.42 grams), 0.1031 grams of PMT, 0.0036 grams of TBA-Oxalate, 0.3094 grams of PAG 1, 10.0% PGMEA solution of KF-353A surfactant (0.0619 grams) and PGMEA 5.13 grams. Thus, a photoresist composition with a solid content of 41.25% by weight was prepared.

The photoresist composition was spin-coated on a silicon wafer substrate, soft-baked at 120° C./180 sec to obtain a film with 12.0 μm thickness. Next, through a pattern mask for measuring resolution, the coated film was exposed by an ASML i-line stepper (NA=0.48, δ=0.55), followed by post-exposure-bake (PEB) of 100° C./60 sec, and then developed with AZ® 300MIF developer (2.38% TMAH, tetramethylammonium hydroxide aqueous solution) for 60 sec with 2 puddles (2×60 sec). The dose-to-print of 4.0 μm L/S (line/space) resolved at 140 mJ/cm².

### Photoresist Example 9, i-line

A CAR composition was made by dissolving 47.1% PGMEA solution of Alnovol SPN560 FAST (7.47 grams), 47.5% PGMEA solution of Alnovol SPN560 SLOW (1.71 grams), 56.0% PGMEA solution of AE6 Polymer (6.86 grams), 0.084 grams of PMT, 0.0029 grams of TBA-Oxalate, 0.1383 grams of PAG 10, 10.0% PGMEA solution of KF-353A surfactant (0.0504 grams) and PGMEA 3.69 grams. Thus, a photoresist composition with a solid content of 42.00% by weight was prepared.

The photoresist composition was spin-coated on a silicon wafer substrate, soft-baked at 120° C./180 sec to obtain a film with 12.0 μm thickness. Next, through a pattern mask for measuring resolution, the coated film was exposed by an ASML i-line stepper (NA=0.48, δ=0.55), followed by post-exposure-bake (PEB) of 100° C./60 sec, and then developed with AZ© 300MIF developer (2.38% TMAH, tetramethylammonium hydroxide aqueous solution) for 60 sec with 2 puddles (2×60 sec). The dose-to-print of 4.0 μm L/S (line/space) resolved at 100 mJ/cm².

### Photoresist Example 10, i-line

A CAR composition was made by dissolving 46.8% PGMEA solution of Alnovol SPN560 FAST (17.52 grams), 47.4% PGMEA solution of Alnovol SPN560 SLOW (3.96 grams), 55.9% PGMEA solution of AE6 Polymer (16.03 grams), 0.0118 grams of MTA, 0.0069 grams of TBA-Oxalate, 0.5299 grams of PAG 7, 10.0% PGMEA solution of KF-353A surfactant (0.1176 grams) and PGMEA 11.82 grams. Thus, a photoresist composition with a solid content of 3⁹0.2% by weight was prepared.

The photoresist composition was spin-coated on a silicon wafer substrate, soft-baked at 120° C./180 sec to obtain a film with 12.0 μm thickness. Next, through a pattern mask for measuring resolution, the coated film was exposed by an ASML i-line stepper (NA=0.48, δ=0.55), followed by post-exposure-bake (PEB) of 100° C./60 sec, and then developed with AZ® 300MIF developer (2.38% TMAH, tetramethylammonium hydroxide aqueous solution) for 60 sec with 2 puddles (2×60 sec). The dose-to-print of 4.0 μm L/S (line/space) resolved at 200 mJ/cm².

### Photoresist Example 11, i-line

A CAR composition was made by dissolving 46.8% PGMEA solution of Alnovol SPN560 FAST (17.60 grams), 47.4% PGMEA solution of Alnovol SPN560 SLOW (3.96 grams), 55.9% PGMEA solution of AE6 Polymer (16.03 grams), 0.0118 grams of MTA, 0.0069 grams of TBA-Oxalate, 0.49 grams of PAG 5, 10.0% PGMEA solution of KF-353A surfactant (0.1176 grams) and PGMEA 11.77 grams. Thus, a photoresist composition with a solid content of 39.2% by weight was prepared.

The photoresist composition was spin-coated on a silicon wafer substrate, soft-baked at 120° C./180 sec to obtain a film with 12.0 μm thickness. Next, through a pattern mask for measuring resolution, the coated film was exposed by an ASML i-line stepper (NA=0.48, δ=0.55), followed by post-exposure-bake (PEB) of 100° C./60 sec, and then developed with AZ® 300MIF developer (2.38% TMAH, tetramethylammonium hydroxide aqueous solution) for 60 sec with 2 puddles (2×60 sec). The dose-to-print of 4.0 μm L/S (line/space) resolved at 280 mJ/cm².

### Photoresist Example 12, i-line

A CAR composition was made by dissolving 47.1% PGMEA solution of Alnovol SPN560 FAST (9.60 grams), 47.5% PGMEA solution of Alnovol SPN560 SLOW (9.52 grams), 1.07 grams of HMMM, 0.25 grams of DML-POP, 0.28 grams of PAG 1, 10.0% PGMEA solution of TMEEA (0.1599 grams), 10.0% PGMEA solution of KF-353A surfactant (0.05 grams) and PGMEA 4.07 grams. Thus, a photoresist composition with a solid content of 42.65% by weight was prepared.

The photoresist composition was spin-coated on a silicon wafer substrate, soft-baked at 110° C./180 sec to obtain a film with 10.0 μm thickness. Next, through a pattern mask for measuring resolution, the coated film was exposed by an ASML i-line stepper (NA=0.48, δ=0.55), followed by post-exposure-bake (PEB) of 120° C./60 sec, and then developed with AZ© 300MIF developer (2.38% TMAH, tetramethylammonium hydroxide aqueous solution) for 50 sec with 3 puddles (3×50 sec). The dose-to-print of 5.0 μm L/S (line/space) resolved at 450 mJ/cm².

Table 6 (FIG. 25) demonstrates the lithographic performance of Photoresist Example 8 on Si and Cu substrate and Photoresist Example 9 (PAG 10) on Si substrate. (PAG 10 shows comparable photospeed with PAG 1 by half-molar loading.)

Table 7 (FIG. 26) demonstrates the lithographic performance of Photoresist Example 12. (Photoresist Example 12 shows a faster photospeed than its similar formulation Photoresist Examples 1)

1. A covalent compound comprising an imide N-(carbonylcarbamido) aryl sulfate moiety wherein said compound is free of any fluoroalkyls, any perfluoroalkyls or mixtures of fluoroalkyls and perfluoroalkyls.

2. The covalent compound of claim 1 having structure (I), wherein R₁ is a linking group selected from the group consisting of an unsubstituted alkylene, a substituted alkylene, an unsubstituted vinylene, a substituted vinylene, an unsubstituted arylene, and a substituted arylene, where these linking groups are free of any fluoroalkyls, any perfluoroalkyls or mixtures of fluoroalkyls and perfluoroalkyls, and R₂ is an unsubstituted aryl or a substituted aryl free of any fluoroalkyls, any perfluoroalkyls or mixtures of fluoroalkyls and perfluoroalkyls and np is 1, 2, 3, 4 or 5;

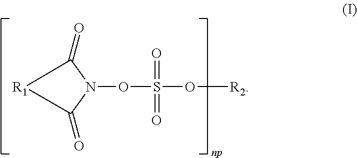

3. The covalent compound of claim 2, wherein R₁ is an unsubstituted alkylene or a substituted alkylene and said compound has structure (Ia), wherein R₁a, R₁b, R′₁a and R′₁b are independently selected from the group consisting of a hydrogen atom, a C-1 to C-18 alkyl, a C-1 to C-18 alkoxy, an unsubstituted aryl, a substituted aryl and mixtures thereof, and np is 1, 2, 3, 4 or 5;

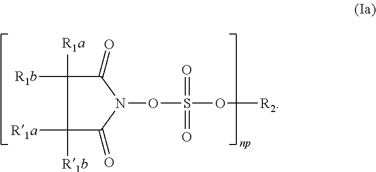

4. The covalent compound of claim 2, wherein R₁ is an unsubstituted alkylene or a substituted alkylene and said compound has structure (Ib), wherein R₁a, R₁b, R₁c, R′₁a, R′₁b, R′₁c, are independently selected from the group consisting of a hydrogen atom, Cl, Br, I, an unsubstituted C-1 to C-18 alkyl, a substituted C-1 to C-18 alkyl, an unsubstituted C-1 to C-18 alkoxy, a substituted C-1 to C-18 alkoxy, an unsubstituted aryl and a substituted aryl and np is 1, 2, 3, 4 or 5;

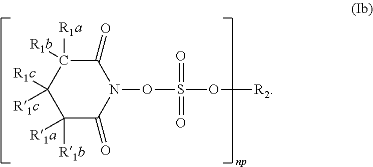

5. The covalent compound of claim 2, wherein R₁ is an unsubstituted alkylene or a substituted alkylene and said compound has structure (Ic), wherein R₁a, R₁b, R₁c, R′₁a, R′₁b, R′₁c, are independently selected from the group consisting of a hydrogen atom, a C-1 to C-18 alkyl, a C-1 to C-18 alkoxy, an unsubstituted aryl, a substituted aryl and mixtures thereof, and np is 1, 2, 3, 4 or 5;

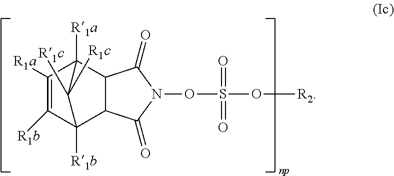

6. The covalent compound of claim 2, wherein R₁ is an unsubstituted vinylene or a substituted vinylene and said compound has structure (Id), wherein Rid and R′₁d are independently selected from the group consisting of a hydrogen atom, Cl, Br, I, an unsubstituted C-1 to C-18 alkyl, a substituted C-1 to C-18 alkyl, an unsubstituted C-1 to C-18 alkoxy, a substituted C-1 to C-18 alkoxy, an unsubstituted aryl, a substituted aryl, and mixtures thereof, and np is 1, 2, 3, 4 or 5;

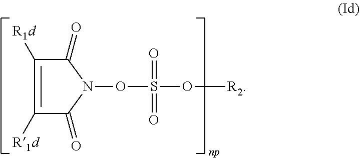

7. The covalent compound of claim 2, wherein R₁ is an unsubstituted arylene or a substituted arylene and said compound has structure (Ie), wherein Rar₁, Rar₂, Rar₃, and Rar₄, are independently selected from the group consisting of a hydrogen atom, F, Cl, Br, I, an unsubstituted C-1 to C-18 alkyl, a substituted C-1 to C-18 alkyl, an unsubstituted C-1 to C-18 alkoxy, a substituted C-1 to C-18 alkoxy, an unsubstituted aryl, a substituted aryl, and mixtures thereof, and np is 1, 2, 3, 4 or 5;

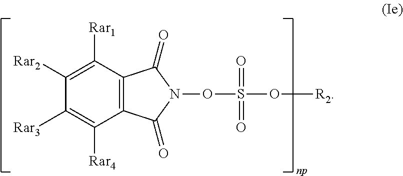

8. The covalent compound of claim 2, wherein R₁ is an unsubstituted arylene or a substituted arylene, which is selected from a substituted fused polycyclic aromatic hydrocarbon moiety, or an unsubstituted fused polycyclic aromatic hydrocarbon moiety.

9. The covalent compound of claim 2, wherein R₁ is an unsubstituted arylene or a substituted arylene, which is selected from a substituted fused polycyclic aromatic hydrocarbon moiety, or an unsubstituted fused aromatic hydrocarbon moiety selected from the group consisting of a naphthalene moiety, an anthracene moiety, a phenanthrene moiety, a phenalene moiety, a tetracene moiety, a chrysene moiety a triphenylene moiety, a pyrene moiety, a pentacene moiety, and a perylene moiety, further wherein these moieties may be substituted or unsubstituted.

10. (canceled)

11. The covalent compound of claim 2, wherein R₁ is an unsubstituted arylene or a substituted arylene, which is selected from a substituted fused polycyclic aromatic hydrocarbon moiety, or an unsubstituted fused aromatic hydrocarbon moiety which has structure (If), where Rar₈, Rar₆, Rar₇, Rar₈, Rar₉, Rar₁₀, are independently selected from a hydrogen atom, F, Cl, Br, I, a substituted aryl, an unsubstituted aryl, an unsubstituted C-1 to C-18 alkyl a substituted C-1 to C-18 alkyl, an unsubstituted C-1 to C-18 alkoxy, a substituted C-1 to C-18 alkoxy an unsubstituted C-1 to C-18 alkylthio, a substituted C-1 to C-18 alkylthio and mixtures thereof, where for substituents comprising C-2 to C-18 unsubstituted alkyl, and substituted C-2 to C-18 alkyl these may optionally comprise within the length of the alkyl moiety, a moiety within their length selected from a carbon to carbon double bond (═), a carbon to carbon triple bond (≡), and a heteroatom comprising moiety selected from —O—, —S—, —C(═O)—, —C(═S)—, —S(═O)₂—, —S(═O)—, —C(═O)—O—, —C(═O)—S—, —O—S(═O)₂—, —O—C(═O)—O—, —C(═O)—NH—, —O—C(═O)—NH—, and —C(═O)—NR—, and mixtures thereof; where R is a C-1 to C-18 alkyl, and np is 1, 2, 3, 4 or 5;

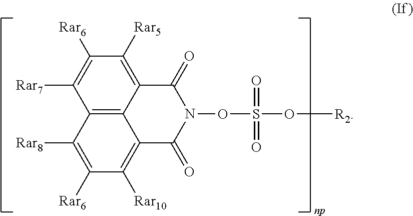

12. The covalent compound of claim 2, wherein R₂ is phenyl or a substituted phenyl.

13. (canceled)

14. The covalent compound of claim 2, wherein R₂ is a substituted phenyl, which when np is 1 it contains 1 to 5 further substituents, when np is 2 it contains 1 to 4 further substituents, when np is 3 it contains 1 to 3 further substituents, when np is 4 it contains 1 to 2 further substituents, when np is 5 it contains 1 further substituent, which are independently selected from the group consisting of −NO₂, —OH, —O-Rw-CN, —F, —Cl, —Br, —I, C-1 to C-18 unsubstituted alkyl, C-1 to C-18 substituted alkyl, —S(═O)₂-Rw, —O—S(═O)₂-Rw, —O—S(═O)₂—O-Rw1, —O—S(═O)-Rw, —O—S(═O)—O-Rw1, —C(═O)—H, —C(═O)-Rw, —C(═O)—O-Rw, —C(═O)—S-Rw, —O—C(═O)—S-Rw, —O—C(═O)-Rw, —O—C(═O)—O-Rw, —S-Rw1, —C(═S)-Rw, —C(═S)—O-Rw, —C(═S)—S-Rw, —O—C(═S)—S-Rw, an alkynyl, an alkenyl, and mixtures thereof, where Rw is a C-1 to C-18 unsubstituted or substituted alkyl, or a substituted or unsubstituted aryl, and Rw1 is an unsubstituted or substituted aryl.

15.-17. (canceled)

18. The covalent compound of claim 1, where said compound has structure (Ig), where Rew, Rew₁, Rew₂, Rew₃, and Rew₄, are independently selected from a hydrogen atom, from —NO₂, —OH, —O-Rw, —CN, —F, —Cl, —Br, —I, C-1 to C-18 unsubstituted alkyl, C-1 to C-18 substituted alkyl, —S(═O)₂-Rw, —O—S(═O)₂-Rw, —O—S(═O)₂—O-Rw1, —O—S(═O)-Rw, —O—S(═O)—O-Rw1, —C(═O)—O-Rw, —C(═O)—H, —C(═O)—S-Rw, —O—C(═O)—S-Rw, —O—C(═O)-Rw, —O—C(═O)—O-Rw, —S-Rw1, —C(═S)-Rw, —C(═S)—O-Rw, —C(═S)—S-Rw, —O—C(═S)—S-Rw, an alkynyl, an alkenyl, a naphthaleneimido sulfate moiety of structure (Iga) where ** represents the attachment point of the naphthaleneimido sulfate of structure (Iga), and mixtures thereof, where Rw is a C-1 to C-18 unsubstituted or substituted alkyl, or a substituted or unsubstituted aryl, and Rw1 is an unsubstituted or substituted aryl, and where Rar₈, Rar₆, Rar₇, Rar₈, Rar₉, Rar₁₀, are independently selected from a hydrogen atom, F, Cl, Br, I, a substituted aryl, an unsubstituted aryl, an unsubstituted C-1 to C-18 alkyl, a substituted C-1 to C-18 alkyl, an unsubstituted C-1 to C-18 alkoxy, a substituted C-1 to C-18 alkoxy, an unsubstituted C-1 to C-18 alkylthio, a substituted C-1 to C-18 alkylthio, and mixtures thereof, where for substituents comprising C-2 to C-18 unsubstituted alkyl, and substituted C-2 to C-18 alkyl these may optionally comprise, within the length of the alkyl moiety, a moiety within their length selected from a carbon to carbon double bond (═), a carbon to carbon triple bond (≡), and a heteroatom comprising moiety selected from —O—, —S—, —C(═O)—, —C(═S)—, —S(═O)₂—, —S(═O)—, —C(═O)—O—, —C(═O)—S—, —O—S(═O)₂—, —O—C(═O)—O—, —C(═O)—NH—, —O—C(═O)—NH—, and —C(═O)—NR—, and mixtures thereof; where R is a C-1 to C-18 alkyl;

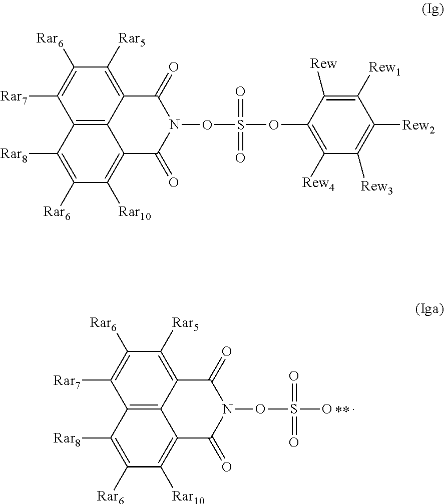

19. (canceled)

20. The covalent compound of claim 1, where said compound has structure (Ii), wherein X is 0 or S, Ri₁ is a C-1 to C-18 alkyl substituted or unsubstituted alkyl and mixtures thereof, where for C-2 to C-18 unsubstituted alkyl, and substituted C-2 to C-18 alkyl these may optionally comprise a moiety within their length selected from a carbon to carbon double bond (═), a carbon to carbon triple bond (≡), and a heteroatom comprising moiety selected from —O—, —S—, —C(═O)—, —C(═S)—, —S(═O)₂—, —S(═O)—, —C(═O)—O—, —C(═O)—S—, —O—S(═O)₂—, —O—C(═O)—O—, —C(═O)—NH—, —O—C(═O)—NH—, and —C(═O)—NR—, and mixtures thereof, where R is a C-1 to C-18 alkyl; and where, Rew, Rew₁, Rew₂, Rew₃, and Rew₄, are independently selected from the group consisting of a hydrogen atom, —NO₂, —OH, —O-Rw, —CN, —F, —Cl, —Br, —I, a C-1 to C-18 unsubstituted alkyl, a C-1 to C-18 substituted alkyl, phenyl, a substituted phenyl, —S(═O)₂-Rw, —O—S(═O)₂-Rw, —O—S(═O)₂—O-Rw1, —O—S(═O)-Rw, —O—S(═O)—O-Rw1, —C(═O)—H, —C(═O)-Rw, —C(═O)—O-Rw, —C(═O)—S-Rw, —O—C(═O)—S-Rw, —O—C(═O)-Rw, —O—C(═O)—O-Rw, —S-Rw1, —C(═S)-Rw, —C(═S)—O-Rw, —C(═S)—S-Rw, —O—C(═S)—S-Rw, an alkynyl, an alkenyl, a substituted naphthaleneimido sulfate moiety of structure (Ii-1), and mixtures thereof, where Rw is a C-1 to C-18 substituted or unsubstituted alkyl or an unsubstituted or substituted aryl and Rw1 is an unsubstituted or substituted aryl and ** represents the attachment point of the substituted naphthaleneimido sulfate moiety of structure (Ii-1);

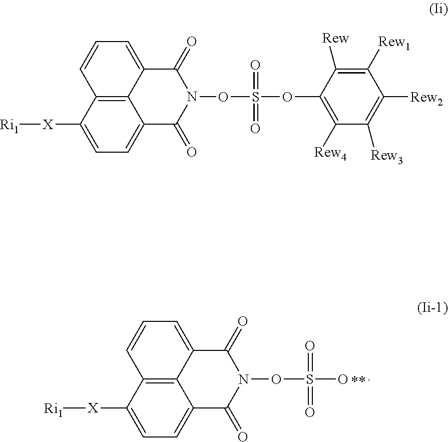

21.-26. (canceled)

27. The covalent compound of claim 1, where said compound has structure (Ij), wherein X is 0 or S, Ri₁ is a C-1 to C-18 substituted alkyl or unsubstituted alkyl and mixtures thereof, where for C-2 to C-18 unsubstituted alkyl, and substituted C-2 to C-18 alkyl these may optionally comprise a moiety within their length selected from a carbon to carbon double bond (═), a carbon to carbon triple bond (≡-), and a heteroatom comprising moiety selected from —O—, —S—, —C(═O)—, —C(═S)—, —S(═O)₂—, —S(═O)—, —C(═O)—O—, —C(═O)—S—, —O—S(═O)₂—, —O—C(═O)—O—, —C(═O)—NH—, —O—C(═O)—NH—, and —C(═O)—NR—, and mixtures thereof, where R is a C-1 to C-18 alkyl; where, Rew, Rew₁, Rew₂, Rew₃, and Rew₄, are independently selected from the group consisting of a hydrogen atom, —NO₂, —OH, —O-Rw, —CN, —F, —Cl, —Br, —I, a C-1 to C-18 unsubstituted alkyl, a C-1 to C-18 substituted alkyl, phenyl, a substituted phenyl, —S(═O)₂-Rw, —O—S(═O)₂-Rw, —O—S(═O)₂—O-Rw1, —O—S(═O)-Rw, —O—S(═O)—O—Rw1, —C(═O)—H, —C(═O)-Rw, —C(═O)—O-Rw, —C(═O)—S-Rw, —O—C(═O)—S—Rw, —O—C(═O)-Rw, —O—C(═O)—O-Rw, S-Rw1, —C(═S)-Rw, —C(═S)—O-Rw, —C(═S)—S-Rw, —O—C(═S)—S-Rw, an alkynyl, an alkenyl, a substituted naphthaleneimido sulfate moiety of structure (Ij-1), and mixtures thereof, where Rw is a C-1 to C-18 substituted or unsubstituted alkyl or an unsubstituted or substituted aryl and Rw1 is an unsubstituted or substituted aryl and ** represents the attachment point of the substituted naphthaleneimido sulfate moiety of structure (Ij-1);

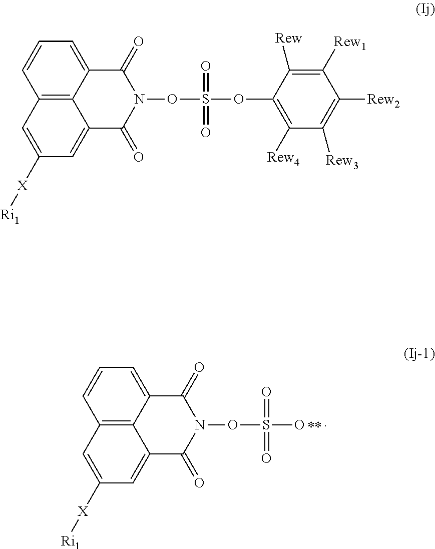

28.-33. (canceled)

34. The covalent compound of claim 1, where said compound has structure (Ik), wherein X₁ is direct valence bond, or a C-1 to C-18 alkylene spacer, Ro and Rol are independently selected from a hydrogen atom, a C-1 to C-18 unsubstituted alkyl, a substituted C-1 to C-18 alkyl, and mixtures thereof, where for C-2 to C-18 unsubstituted alkyl, and substituted C-2 to C-18 alkyl these may optionally comprise a moiety within their length selected from a carbon to carbon double bond (═), a carbon to carbon triple bond (≡), and a heteroatom comprising moiety selected from —O—, —S—, —C(═O)—, —C(═S)—, —S(═O)₂—, —S(═O)—, —C(═O)—O—, —C(═O)—S—, —O—S(═O)₂—, —O—C(═O)—O—, —C(═O)—NH—, —O—C(═O)—NH—, and —C(═O)—NR—, and mixtures thereof, where R is a C-1 to C-18 alkyl; and where, Rew, Rew₁, Rew₂, Rew₃, and Rew₄, are independently selected from the group consisting of a hydrogen atom, —NO₂, —OH, —O-Rw, —CN, —F, —Cl, —Br, —I, a C-1 to C-18 unsubstituted alkyl, a C-1 to C-18 substituted alkyl, phenyl, a substituted phenyl, —S(═O)₂-Rw, —O—S(═O)₂-Rw, —O—S(═O)₂—O-Rw1, —O—S(═O)-Rw, —O—S(═O)—O-Rw1, —C(═O)—H, —C(═O)-Rw, —C(═O)—O-Rw, —C(═O)—S-Rw, —O—C(═O)—S—Rw, —O—C(═O)-Rw, —O—C(═O)—O-Rw, —S-Rw1, —C(═S)-Rw, —C(═S)—O-Rw, —C(═S)—S-Rw, —O—C(═S)—S-Rw, an alkynyl, an alkenyl, a substituted naphthaleneimido sulfate moiety of structure (Ik-1), and mixtures thereof, where Rw is a C-1 to C-18 substituted or unsubstituted alkyl or an unsubstituted or substituted aryl and Rw1 is an unsubstituted or substituted aryl and ** represents the attachment point of the substituted naphthaleneimido sulfate moiety of structure (Ik-1);

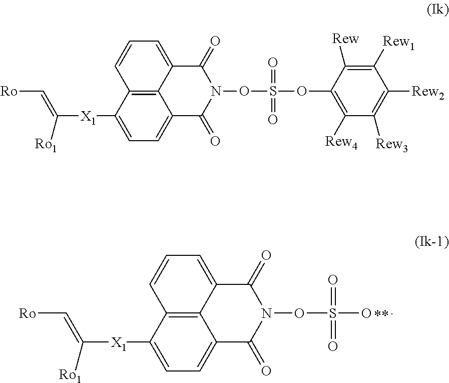

35. The covalent compound of claim 1, where said compound has structure (Il), wherein X₁ is direct valence bond, or a C-1 to C-18 alkylene spacer, Ro and Rol are independently selected from a hydrogen atom, a C-1 to C-18 unsubstituted alkyl, a substituted C-1 to C-18 alkyl, and mixtures thereof, where for C-2 to C-18 unsubstituted alkyl, and substituted C-2 to C-18 alkyl these may optionally comprise a moiety within their length selected from a carbon to carbon double bond (═), a carbon to carbon triple bond (≡), and a heteroatom comprising moiety selected from —O—, —S—, —C(═O)—, —C(═S)—, —S(═O)₂—, —S(═O)—, —C(═O)—O—, —C(═O)—S—, —O—S(═O)₂—, —O—C(═O)—O—, —C(═O)—NH—, —O—C(═O)—NH—, and —C(═O)—NR—, and mixtures thereof, where R is a C-1 to C-18 alkyl; and where, Rew, Rew₁, Rew₂, Rew₃, and Rew₄, are independently selected from the group consisting of a hydrogen atom, —NO₂, —OH, —O-Rw, —CN, —F, —Cl, —Br, —I, a C-1 to C-18 unsubstituted alkyl, a C-1 to C-18 substituted alkyl, phenyl, a substituted phenyl, —S(═O)₂-Rw, —O—S(═O)₂-Rw, —O—S(═O)₂—O-Rw1, —O—S(═O)-Rw, —O—S(═O)—O-Rw 1, —C(═O)—H, —C(═O)-Rw, —C(═O)—O-Rw, —C(═O)—S-Rw, —O—C(═O)—S—Rw, —O—C(═O)-Rw, —O—C(═O)—O-Rw, —S-Rw1, —C(═S)-Rw, —C(═S)—O-Rw, —C(═S)—S-Rw, —O—C(═S)—S-Rw, an alkynyl, an alkenyl, a substituted naphthaleneimido sulfate moiety of structure (Il-1), and mixtures thereof, where Rw is a C-1 to C-18 substituted or unsubstituted alkyl or an unsubstituted or substituted aryl and Rw1 is an unsubstituted or substituted aryl and ** represents the attachment point of the substituted naphthaleneimido sulfate moiety of structure (Il-1);

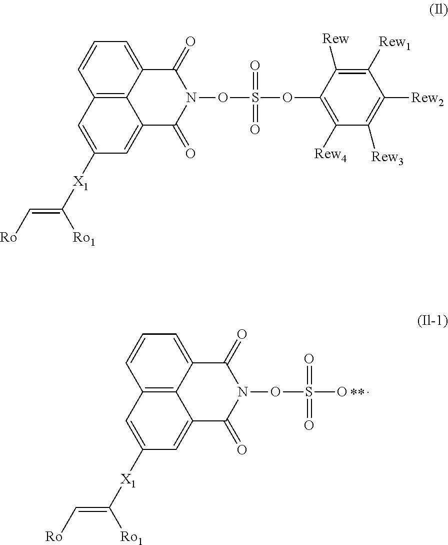

36. The covalent compound of claim 1, where said compound has structure (Im), wherein X₁ is direct valence bond, or a C-1 to C-8 alkylene spacer, Ro₃ is selected from a hydrogen atom, a phenyl, a substituted phenyl, a thiophen-3-yl, a C-1 to C-18 unsubstituted alkyl, a substituted C-1 to C-18 alkyl, and mixtures thereof, where for C-2 to C-18 unsubstituted alkyl, and substituted C-2 to C-18 alkyl these may optionally comprise a moiety within their length selected from a carbon to carbon double bond (═), a carbon to carbon triple bond (≡), and a heteroatom comprising moiety selected from —O—, —S—, —C(═O)—, —C(═S)—, —S(═O)₂—, —S(═O)—, —C(═O)—O—, —C(═O)—S—, —O—S(═O)₂—, —O—C(═O)—O—, —C(═O)—NH—, —O—C(═O)—NH—, and —C(═O)—NR—, and mixtures thereof, where R is a C-1 to C-18 alkyl; and where Rew, Rew₁, Rew₂, Rew₃, and Rew₄, are independently selected from the group consisting of a hydrogen atom, —NO₂, —OH, —O-Rw, —CN, —F, —Cl, —Br, —I, a C-1 to C-18 unsubstituted alkyl, a C-1 to C-18 substituted alkyl, phenyl, a substituted phenyl, —S(═O)₂-Rw, —O—S(═O)₂-Rw, —O—S(═O)₂—O-Rw1, —O—S (═O)-Rw, —O—S(═O)—O-Rw1, —C(═O)—H, —C(═O)-Rw, —C(═O)—O-Rw, —C(═O)—S-Rw, —O—C(═O)—S-Rw, —O—C(═O)-Rw, —O—C(═O)—O-Rw, —S-Rw1, —C(═S)-Rw, —C(═S)—O-Rw, —C(═S)—S-Rw, —O—C(═S)—S-Rw, an alkynyl, an alkenyl, a substituted naphthaleneimido sulfate moiety of structure (Im-1), and mixtures thereof, where Rw is a C-1 to C-18 substituted or unsubstituted alkyl or an unsubstituted or substituted aryl and Rw1 is an unsubstituted or substituted aryl and ** represents the attachment point of the substituted naphthaleneimido sulfate moiety of structure (Im-1);

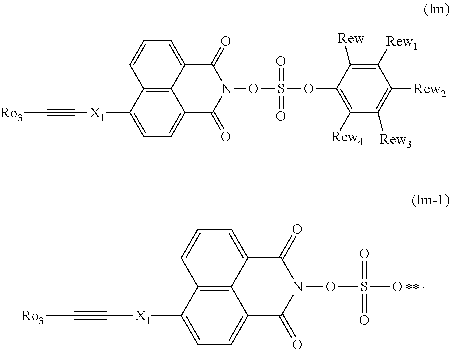

37.-41. (canceled)

42. The covalent compound of claim 1, where said compound has structure (In), wherein X₁ is direct valence bond, or a C-1 to C-8 alkylene spacer, Ro₃ is selected from a hydrogen atom, a phenyl, a substituted phenyl, a thiophen-3-yl, a C-1 to C-18 unsubstituted alkyl, a substituted C-1 to C-18 alkyl, and mixtures thereof, where for C-2 to C-18 unsubstituted alkyl, and substituted C-2 to C-18 alkyl these may optionally comprise a moiety within their length selected from a carbon to carbon double bond (═), a carbon to carbon triple bond (≡), and a heteroatom comprising moiety selected from —O—, —S—, —C(═O)—, —C(═S)—, —S(═O)₂—, —S(═O)—, —C(═O)—O—, —C(═O)—S—, —O—S(═O)₂—, —O—C(═O)—O—, —C(═O)—NH—, —O—C(═O)—NH—, and —C(═O)—NR—, and mixtures thereof, where R is a C-1 to C-18 alkyl; and where, Rew, Rew₁, Rew₂, Rew₃, and Rew₄, are independently selected from the group consisting of a hydrogen atom, —NO₂, —OH, —O-Rw, —CN, —F, —Cl, —Br, —I, a C-1 to C-18 unsubstituted alkyl, a C-1 to C-18 substituted alkyl, phenyl, a substituted phenyl, —S(═O)₂-Rw, —O—S(═O)₂-Rw, —O—S(═O)₂—O-Rw1, —O—S(═O)-Rw, —O—S(═O)—O-Rw 1, —C(═O)—H, —C(═O)-Rw, —C(═O)—O-Rw, —C(═O)—S-Rw, —O—C(═O)—S—Rw, —O—C(═O)-Rw, —O—C(═O)—O-Rw, —S-Rw1, —C(═S)-Rw, —C(═S)—O-Rw, —C(═S)—S-Rw, —O—C(═S)—S-Rw, an alkynyl, an alkenyl, a substituted naphthaleneimido sulfate moiety of structure (In-1), and mixtures thereof, where Rw is a C-1 to C-18 substituted or unsubstituted alkyl or an unsubstituted or substituted aryl and Rw1 is an unsubstituted or substituted aryl and ** represents the attachment point of the substituted naphthaleneimido sulfate moiety of structure (In-1);

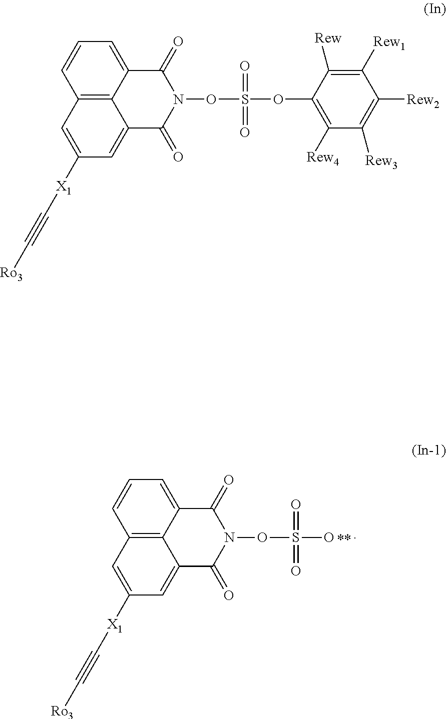

43.-48. (canceled)

49. The covalent compound of claim 1 which has structure (Io), wherein Rnc₁ and Rnc₂ are organic substituents which are free of any fluoroalkyls, any perfluoroalkyls or mixtures of fluoroalkyls and perfluoroalkyls which are independently selected from an unsubstituted C-1 to C-18 alkyl, a substituted C-1 to C-18 alkyl, an unsubstituted aryl, a substituted aryl and a mixture thereof, and R₂ is an unsubstituted aryl or a substituted aryl which is also free of any fluoroalkyls, any perfluoroalkyls or mixtures of fluoroalkyls and perfluoroalkyls and np is 1, 2, 3, 4 or 5;

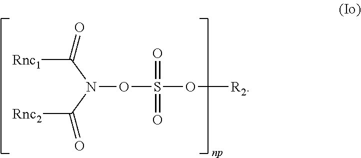

50. (canceled)

51. (canceled)

52. A positive chemically amplified photoresist composition developable in aqueous base, comprising,
1) a PAG component which is a covalent compound of claim 1,
2) at least one polymer, comprising one or more repeat units with at least one acid cleavable group, which cleave upon the action of photogenerated acid from the PAG component which yields upon removal of the acid cleavable group base solubilizing groups which renders the polymer soluble in aqueous base developer,
3) an optional resin component which is soluble in 0.26 N aqueous TMAH,
4) an optional DNQ PAC component,
5) an optional glycidyl hydroxy benzoic acid condensate additive,
6) an optional base component,
7) an optional photobleaching dye component,
8) an optional sensitizer,
9) an optional different type of PAG component,
10) an optional surfactant component,
11) an organic spin coating solvent
12) An optional thiol derivative component where the thiol moiety is attached to an sp2 carbon which is part of the ring, wherein said thiol derivative is selected from the group consisting of heterocyclic thiol compound and an aryl thiol compound.

53.-62. (canceled)

63. The process of forming a positive image with positive photoresist exposed to radiation, comprising step i) to v);
i) coating the composition of claim 52 on a substrate-
ii) baking said coated film to form a baked film,
iii) exposing regions of the baked film through a mask with radiation, forming exposed and unexposed regions,
iv) an optional post exposure baking step,
v) developing away with an aqueous base, said exposed region, forming a positive image on said substrate.

64. A negative chemically amplified photoresist composition comprising
1a) a PAG component which is a covalent compound of claim 1,
2a) a photoresist resin soluble in aqueous base which undergoes chemically amplified crosslinking in the presence of a photogenerated acid,
3a) an optional resin component which is soluble in 0.26 N aqueous TMAH,
4a) an optional crosslinking component,
5a) an optional DNQ-PAC component,
6a) an optional thiol derivative component where the thiol moiety is attached to an sp2 carbon which is part of the ring, wherein said thiol derivative is selected from the group consisting of heterocyclic thiol compound and an aryl thiol compound,
7a) an optional base component,
8a) an optional photobleaching dye component,
9a) an optional dye,
10a) an optional different type of PAG component
11a) an optional sensitizer component,
12a) an optional surfactant component.
13a) an organic spin coating solvent.

65.-74. (canceled)

75. The process of forming negative image with a negative photoresist by exposure to radiation, comprising step ia) to va)
ia) coating the composition of claim 64, on a substrate,
iia) baking said coated film to form a baked film,
iiia) exposing regions of the baked film through a mask with radiation, forming exposed and unexposed regions,
iva) an optional post exposure baking step,
va) developing away the unexposed regions forming a negative image on said substrate.

76. (canceled)
